SEQUENCE LISTING (12 sequences)

SEQ ID NO: 1 - protein, 10 aa, synthetic construct
EQKLISEEDL

SEQ ID NO: 2 - protein, 8 aa, synthetic construct
DYKDDDDK

SEQ ID NO: 3 - protein, 740 aa, synthetic construct
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKLMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 4 - protein, 159 aa, synthetic construct
MALPVTALLLPLALLLHAARPEQKLISEEDLMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVS

SEQ ID NO: 5 - protein, 306 aa, synthetic construct
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 6 - protein, 248 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 7 - protein, 15 aa, synthetic construct
ILDYSFTGGAGRDIP

SEQ ID NO: 8 - protein, 12 aa, synthetic construct
RKRRRQLCIQKL

SEQ ID NO: 9 - protein, 4 aa, synthetic construct
GGGS

SEQ ID NO: 10 - protein, 5 aa, synthetic construct
  VARIANT 5: note = X is Q, A, E or S.
GGGGX

SEQ ID NO: 11 - protein, 5 aa, synthetic construct
GGGGS

SEQ ID NO: 12 - protein, 695 aa, synthetic construct
MALPVTALLLPLALLLHAARPDYKDDDDKMAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVSSPFTILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

(19) United States
(12) Patent Application Publication
(10) Pub. No.: US 2026/0297539 A1
(43) Pub. Date: Oct. 1, 2026

(54) SYNTHETIC NOTCH RECEPTORS FOR USE IN CUSTOMIZED SPATIAL CONTROL OF MULTIPLE GENE EXPRESSIONS AND USES THEREOF

(71) Applicants: University of Southern California, Los Angeles, CA (US); The Regents of the University of California, Oakland, CA (US)

(72) Inventors: Nathan Cho, Los Angeles, CA (US); Stephanie Do, Los Angeles, CA (US); Mher Garibyan, Los Angeles, CA (US); Tyler Hoffman, Oakland, CA (US); Song Li, Oakland, CA (US); Alex March, Los Angeles, CA (US); Megan McCain, Los Angeles, CA (US); Leonardo Morsut, Los Angeles, CA (US)

(73) Assignees: University of Southern California, Los Angeles, CA (US); The Regents of the University of California, Oakland, CA (US)

(21) Appl. No.: 19/479,856

(22) PCT Filed: May 8, 2024
(86) PCT No.: PCT/US2024/028403

§ 371 Date: Oct. 29, 2025

Related U.S. Application Data
(60) Provisional application No. 63/464,850, filed on May 8, 2023.

(51) Int. Cl. C12N 5/071; C07K 14/705; C07K 14/78

(52) U.S. Cl. CPC C12N 5/0697; C07K 14/705; C07K 14/78; C07K 2319/715; C12N 2501/42; C12N 2501/60; C12N 2502/1335; C12N 2502/28; C12N 2506/1307; C12N 2510/00; C12N 2513/00; C12N 2533/52; C12N 2533/54; C12N 2535/10; C12N 2537/10

(57) ABSTRACT

Synthetic Notch receptors can be activated by ligands presented in different ways from a material, thereby serving as a general platform for generating new user-defined material-to-cell signaling pathways. Synthetic signals can be embedded directly into ECM proteins via genetic engineering of ECM-producing fibroblasts, on materials in 2D via microcontact printing or other micropatterning methods, and in 3D with immobilization such as covalent linking to hydrogels, in user-defined spatial patterns. Hence these material-to-cell pathways were used to spatially control activation of user-defined genetic programs in 2D and 3D. Since multiple synthetic pathways with different specificity can be engineered in a single cell, multicellular population can be patterned in up to three spatial domains of gene expression. We co-transdifferentiated fibroblasts into skeletal muscle or endothelial cell precursors in user-defined spatial patterns. These material-to-genetic engineering approaches provide a new avenue for spatial control of cellular behaviors in mammalian multicellular systems.

### CROSS-REFERENCE TO RELATED APPLICATIONS

This application includes a claim of priority under 35 U.S.C. § 119(e) to U.S. provisional patent application No. 63/464,850, filed May 8, 2023, the entirety of which is hereby incorporated by reference.

### STATEMENT REGARDING FEDERALLY SPONSORED RESEARCH OR DEVELOPMENT

This invention was made with government support under 2034495 awarded by the National Science Foundation and GM138256, awarded by the National Institutes of Health. The government has certain rights in the invention.

### REFERENCE TO SEQUENCE LISTING

This application contains a Sequence Listing submitted as a computer readable form named “065715_000156WO2T_SequenceListing.xml”, having a size in bytes of 14,024 bytes, and created on May 8, 2024. The information contained in this computer readable form is hereby incorporated by reference in its entirety.

### FIELD OF INVENTION

This invention relates to the field of tissue engineering with spatial control, and particularly relating to the use of synthetic Notch receptor.

### BACKGROUND

A fundamental goal in the emerging area of synthetic morphogenesis and tissue engineering is the design and spatially control of gene expression patterns within a multicellular construct.

Intricate patterns of gene expression control the proper organization and physiology of cells, tissues, and organs are a hallmark of complex multicellular systems. Individual cells express genetic networks that drive or support cell fate commitment, and functional behaviors like motility and proliferation. During embryonic development for example, initially uniform cell ensembles activate genetic networks in designated spatial regions to generate tissues with distinct geometrical patterns. The spatial organization of cells within a tissue endows them to coordinate and accomplish complex functions, such as absorption and contractility. In vivo, these spatial domains of gene expression are driven by genetically-encoded communication networks involving intracellular, intercellular, cell-to-extracellular matrix (ECM) and ECM-to-cell components. Several of these networks are active in organoids in vitro, which self-organize and replicate select microscale architectural features similar to native tissues. However, genetic networks in organoids are spatially activated in an autonomous way and some genetic networks fail to activate at all, leading to heterogeneity and stunted tissue structures. Because self-organization is convoluted with differentiation and other complex cell behaviors, in vitro methods for arbitrarily engineering and interrogating spatial gene expression patterns and their impact would augment our understanding of biological systems. Advanced technologies for spatially controlling gene expression would also enable tissues to be engineered with user-defined cellular compositions and geometries, which would be impactful for the fields of regenerative medicine, Organs on Chips, and lab-grown protein-rich food sources.

Classically, tissue engineers have focused on influencing cell differentiation and behavior using endogenous cell surface receptors. For example, natural ligands have been presented to cells in user-defined spatial arrangements to control adhesion, alignment, or differentiation. As this approach relies on engagement of endogenous receptors, such as integrins, stereotyped and often complex behaviors are activated in responding cells. However, with these approaches, users are confined to the limited library of endogenous ligands and receptors and their pre-existing downstream pathways, many of which are multifaceted with ambiguous outcomes. Furthermore, cellular ligand presentation has the disadvantage that control over ligand presentation geometries necessitates control of sender cell location, making the problem circular.

Therefore, it is an object of the present invention to provide a system to present signals to cells in a multicellular construct in a spatially controlled fashion and/or to modulate predetermined genes.

It is another object of the present invention to provide methods for using and methods for preparing the system that present signals at defined spatial resolution to activate or engage with cells in a spatially controlled fashion.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide a system or combination, which includes: (i) a ligand, a fusion protein, a first cell, or a combination thereof, positioned in a pattern on/in a substrate, wherein the fusion protein comprises the ligand and another polypeptide, and the first cell secrete or express on cell surface the ligand or the fusion protein, or a combination thereof; (ii) a second cell expressing a synthetic receptor, wherein the synthetic receptor specifically binds the ligand, the fusion protein, or the first cell via the secreted or expressed ligand or fusion protein; and optionally (iii) the substrate.

In some embodiment, a synthetic receptor comprises synthetic Notch receptor, wherein the synthetic Notch receptor does not bind its naturally-occurring ligand Delta and the synthetic Notch receptor comprises, in covalent linkage: a) an extracellular domain comprising an antigen-specific targeting domain; b) a Notch regulatory region comprising a ligand-inducible proteolytic cleavage site; and c) an intracellular domain, heterologous to the Notch regulatory region, comprising a transcriptional activator, wherein the transcriptional activator replaces a naturally-occurring intracellular notch domain. In various aspects, binding of the antigen-specific targeting domain to the ligand, the fusion protein, or the first cell induces cleavage at the Notch regulatory region, thereby releasing the intracellular domain.

In some embodiments, a system or combination include: (i) two or more different ligands, two or more different fusion proteins each comprising a different ligand and a same or different extracellular matrix polypeptide, or two or more different populations of cells expressing on cell surface the different ligands or the different fusion proteins, each being positioned in a different sub-pattern or a same pattern and collectively forming the predetermined pattern on the surface of or inside the substrate; (ii) two or more different populations of cells each expressing a different synthetic Notch receptor capable of specifically binding to the different ligands.

In some embodiments, a system or combination further include (iv) the culture surface, wherein the second cell is cultured thereon.

In some embodiments, a substrate is an elastomeric stamp, microparticles, a polymeric scaffold or hydrogel, a coverslip, or a microfluidic device; optionally the coverslip having micropatterns of the ligands deposited via the elastomeric stamp or microfluidic device.

In some embodiments, a pattern is a micropattern, having an interspace between neighboring elevations of about 100 μm or between 100 μm and 1000 μm.

Various embodiments provide methods for inducing differentiation or transdifferentiation of a cell in a predetermined pattern, and the methods include: (1) genetically expressing a synthetic Notch receptor in a cell, wherein the cell contains a gene encoding a differentiation or transdifferentiation agent whose expression is driven by a transcriptional activator, and wherein the synthetic Notch receptor comprises an extracellular domain that specifically binds a ligand, a Notch receptor polypeptide comprising a ligand-inducible proteolytic cleavage site, and an intracellular domain comprising the transcriptional activator; (2) providing (e.g., photocrosslinking) the ligand, the fusion protein, or the sender cell on the surface of or in the substrate in the predetermined pattern; and (3) contacting the cell expressing the synthetic Notch receptor with the substrate having the predetermined pattern of the ligand, a fusion protein comprising the ligand and an extracellular matrix polypeptide, or a sender cell expressing on sender cell surface the ligand or secreting the ligand or the fusion protein. In various implementations, binding between the cell expressing the synthetic Notch receptor and the ligand, the fusion protein, or the sender cell induces cleavage of the synthetic Notch receptor, thereby releasing the intracellular domain and causing the transcriptional activator to induce expression of the differentiation or transdifferentiation agent in the cell, thereby differentiating or transdifferentiating the cell in the predetermined pattern; and wherein the transcriptional activator when uncleaved from the synthetic Notch receptor does not induce expression of the differentiation agent, and the transcriptional activator is substantially uncleaved when the cell is not in contact with the ligand, the fusion protein, or the sender cell.

In some embodiments, a differentiation or transdifferentiation agent comprises a transcription factor, such as myoblast determination protein 1 (MyoD), ETS variant transcription factor 2 (ETV2), atonal bHLH transcription factor 1, NK3 homeobox 1 (NKX3-1), SRY-box transcription factor 9 (SOX-9), or an isoform thereof.

In preferred embodiments, a method is provided for differentiation or transdifferentiation of a quantity of the cells into two or more different types of cells, wherein the genetically expressing comprises genetically co-expressing two or more different synthetic Notch receptors in the cells, wherein the two or more different synthetic Notch receptors differ at least by the extracellular domain in specifically binding a different ligand and by the transcriptional activator in driving expression of a different differentiation or transdifferentiation agent, which regulates differentiation or transdifferentiation of the cells into respective type of cells.

In some embodiments, the method is for differentiation or co-differentiation of stem cells. In some embodiments, the method is for transdifferentiation or co-transdifferentiation somatic cells.

In some embodiments, a method is provided for transdifferentiation of a quantity of fibroblasts into myoblasts and endothelial cells in a predetermined pattern, and the method includes: (1) genetically co-expressing two different synthetic Notch receptors in a quantity of fibroblasts, optionally embryonic fibroblasts, wherein the two different synthetic Notch receptors each bind a different ligand and thereby releasing a different transcriptional activator, wherein the different transcription activators each drives expression of either one of MyoD or ETV2 in the fibroblasts; (2) providing the different ligands, different fusion proteins comprising either one of the different ligands and an extracellular matrix polypeptide, or different sender cells secreting or expressing on sender cell surface either one of the different ligands or either one of the different fusion proteins, on the surface of or in the substrate in the predetermined pattern; and (3) contacting (e.g., microprinting) the fibroblasts co-expressing the different synthetic Notch receptors with the substrate. In doing so, binding is induced in the predetermined pattern between the fibroblasts and the different ligands, the different fusion proteins, or the different sender cells, and driving expression of the MyoD or the ETV2 in respective fibroblasts, so as to transdifferentiate the fibroblasts into myoblasts and endothelial cells in the predetermined pattern.

Also provided are co-culture systems of cells, which include myoblasts and endothelial cells, and optionally fibroblasts, in a predetermined pattern made from the method disclosed herein. In some aspects, a co-culture system is in the form of a blood vessel-like tissue structure.

Kits are also provided, which may include a substrate; and a ligand or a fusion protein comprising the ligand and an extracellular matrix polypeptide, positioned on/in the substrate. In some embodiments, a kit may further include a polynucleotide encoding a synthetic Notch receptor, wherein the synthetic Notch receptor does not bind its naturally-occurring ligand Delta and instead binds to the ligand.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1H depicts microparticle-conjugated GFP and cell-deposited fibronectin-GFP activate synNotch. (1A) Schematic of GFP conjugation to microparticles and subsequent co-culture with anti-GFP synNotch receiver fibroblasts that activate mCherry reporter gene; fluorescence image of an individual activated receiver fibroblast in the presence of GFP-loaded-microparticles. (1B) Fluorescence microscopy images of synNotch receiver fibroblasts cultured in the presence of 0, 100, and 1000 μg/mL 5 μm GFP-conjugated microparticles or GFP-presenting sender cells following 24 hours culture. Scale bars, 100 μm. (1C) Percent of mCherry expressing cells quantified by image analysis following 24-hour co-culture with GFP microparticles or GFP sender cells. Data represents mean±s.d, n=3-5, p<0.01(**), p<0.001(***), p<0.0001(****). (1D) Schematic of Fibronectin-GFP (FN-GFP) producing fibroblasts, with an miRFP nuclear tag co-cultured with anti-GFP synNotch receiver fibroblast that constitutively express a BFP reporter. (1E) Fluorescence microscopy images of co-cultured FN-GFP cells and synNotch receiver fibroblasts taken after 72 hours of culture. Scale bars, 100 μm. (1F) Schematic of FN-GFP deposition by FN-GFP senders, decellularization, and subsequent reseeding with synNotch fibroblasts. (1G) Top row: schematics of decellularized extracellular matrix (ECM) present on the surface plates before seeding, which is produced by 8 days of culture of different ratios of parental fibroblasts (FN-Parental) and FN-GFP senders (1:0, 1:1 or 0:1, FN-Parental:FN-GFP) prior to decellularization. Bottom: microscope images of anti-GFP synNotch fibroblasts 48 hours following seeding onto the corresponding decellularized ECM. Scale bars, 100 μm. (1H) Flow cytometry mCherry expression of synNotch fibroblasts seeded onto decellularized ECM prepared from varying ratios of FN:Parental and FN-GFP fibroblasts, taken 48 hours following synNotch fibroblast seeding. Arbitrary Units (A.U.). Data represents distribution of individual cell intensity and median value.

FIG. 1I depicts average mCherry intensity of activated reporter cells quantified by image analysis following 24-hour co-culture with 5 μm GFP microparticles of different conjugation concentrations or GFP sender cells. Data represents mean±s.d, n=3-5, p>0.05 (ns), p<0.05 (*), p<0.0001(****).

FIG. 1J includes a schematic and a set of bright field and fluorescence images. The schematic depicts fibronectin-mCherry (FN-mCherry) producing sender fibroblasts, with a miRFP nuclear tag co-cultured with anti-mCherry synNotch/Gal4 receiver fibroblasts that induce the expression of BFP reporter. The brightfield and fluorescence images shows anti-mCherry synNotch reporter fibroblasts uniformly seeded onto a local region of FN-mCherry sender fibroblasts, which were seeded 30 minutes prior within a small droplet (white line indicates region of FN-mCherry seeding). Line plot represents the normalized fluorescence intensity across the x-axis. Scale bars, 2 mm.

FIG. 1K depicts a schematic detailing FN-mCherry sender culture, followed by decellularization, then reseeding with anti-mCherry receivers.

FIGS. 2A-2I depict hydrogel-conjugated ligands robustly and spatially activate reporter transgenes via synNotch. (2A) Schematic of enzymatic GFP conjugation to surface of gelatin hydrogel using transglutaminase (TG) and resulting interactions with synNotch receiver fibroblasts. (2B) Fluorescence microscopy images of anti-GFP/tTA synNotch receiver fibroblasts seeded on gelatin hydrogels conjugated with GFP at 0, 100 or 250 μg/mL. Scale bars, 200 μm. (2C) Flow cytometry analysis of mCherry expression of synNotch fibroblasts 72 hours following seeding onto GFP-conjugated hydrogel surfaces prepared with varying concentrations of GFP (0, 10, 100, 250 μg/mL). Data represents distribution of individual cell intensity and median value. (2D) Schematic of gelatin methacryloyl-methyltetrazine (GelMA-mTz) covalent conjugation to GFP-trans-Cyclooctene (GFP-TCO) and subsequent engineered cell photoencapsulation with UV light. (2E) Z-projected and 3D view of fluorescence microscopy images of anti-GFP synNotch receiver fibroblasts encapsulated within GelMA-mTz hydrogels containing 0 (GelMA) or 50 μg/mL GFP-TCO (GelMA-GFP) at Day 1 and 3. Scale bars, 200 μm. (2F) Percent of mCherry expressing cells quantified by image analysis following 1, 3, and 7 days of culture within GelMA or GelMA-GFP hydrogels. Data represents mean±s.d, n=3-8, p<0.0001(****). (2G) Schematic of engineered cell encapsulation within bi-phasic hydrogel containing spatially localized GFP (2H) Brightfield and fluorescence images of anti-GFP synNotch receiver fibroblasts within bi-phasic GFP hydrogel taken 5 days after encapsulation. (2I) Plot profile of normalized mCherry intensity distribution across the length of the hydrogel 1, 2, 3, and 5 days after encapsulation. Green shaded area indicates the region containing GFP.

FIG. 2J includes a schematic of covalent substitutions to Gelatin, with methacrylate and methyltetrazine, and GFP, with trans-Cyclooctene, to generate gelatin methacryloyl (GelMA) conjugated to GFP (GelMA-GFP), showing functional groups of GelMA-methyltetrazine (GelMA-mTz) with corresponding NMR peaks during each step of gelatin modification, as well as percent substitution and molar content of methacrylate group and methyltetrazine estimated with 1H-NMR.

FIG. 2K includes, in the upper row, image analysis based quantifications of GFP intensity and activated anti-GFP synNotch receiver fibroblasts (mCherry signal) within GelMA-mTz hydrogels containing 0, 12.5, 50, or 100 μg/mL GFP taken up to 14 days (data represents mean±s.d, n=3); and in the lower left, mCherry intensity of encapsulated anti-GFP synNotch mCherry fibroblasts quantified by image analysis following 1, 3, and 7 days of culture within GelMA or GelMA-GFP hydrogels (data represents mean±s.d, n=3-8, p<0.01 (**), p<0.0001(****)), and lower right, quantification of fluorescence images of anti-GFP synNotch fibroblast-laden GelMA hydrogels stained with Live/Dead taken 1, 3, 7, and 14 days after fabrication, wherein live cells are stained with Calcein-AM, dead cells are stained with ethidium homodimer-1 (EtHD-1), at a scale bars, 200 μm.

FIG. 2L depicts a plot profile of normalized GFP intensity distribution across the length of the bi-phasic GelMA hydrogel, where one portion contains GFP, 1, 2, 3, and 5 days after encapsulation.

FIG. 2M depicts Percent of mCherry expressing receiver cells quantified by image analysis following 1, 3, and 7 days of culture with varying ratios of sender cells. Data represents mean±s.d, n=3-5, p<0.05(*), p<0.01(**), p<0.001(***), p<0.0001(****).

FIG. 2N depicts a schematic of fibrinogen-mTz and mCherry-TCO reaction to generate Fibrinogen-mCherry used to encapsulate anti-mCherry reporter fibroblasts with inducible BFP.

FIGS. 3A-3G depicts microcontact printed GFP patterns spatially activate mCherry reporter via synNotch pathway activation. (3A) Visual schematic showing the process of stamp preparation and concurrent coverslip preparation for the microcontact printing of GFP patterns. (3B) Binary mask of the stamp features that contains 100 μm square features with 100 μm gaps and resulting GFP fluorescence image following microcontact printing (3C) Schematic of anti-GFP/tTA synNotch receiver fibroblasts seeded onto GFP-patterned substrate to demonstrate local activation based on the presence of GFP. Portions of the cells were made transparent to visualize the underlying pattern of GFP. (3D) Binary masks and fluorescence microscopy images of 500 μm and 250 μm side square GFP patterns, separated by 250, 350, 500, and 1000 μm gaps, followed by mCherry fluorescence images of anti-GFP synNotch receiver cells two days following seeding. Dotted white lines separate regions with different interspaces and the solid white lines present the location of GFP patterns enlarged in the following panel. (3E) Plot profile of normalized mCherry intensity on 2, 5, and 10 days following seeding onto 500×1000 μm (top), 250×250 μm (bottom) or GFP pattern (square side×interspace). Green indicates the regions containing GFP. (3F) Quantifications of day 2 Pearson's Correlation Coefficient (PCC) comparing Binary Mask with mCherry channel across all patterns. Scrambled binary mask images were compared to day 2 mCherry channels as a negative control. Data represents mean±s.d, n=7-8, not significant p>0.05 (ns), p<0.01(**), p<0.0001(****). (3G) Binary masks, brightfield microscopy images, and GFP fluorescence images of concentric circles and letter patterns of GFP, followed by mCherry fluorescence microscopy images taken two days following seeding onto the GFP pattern. Dotted white rectangles in the brightfield indicate the region of interest shown in higher magnification on the bottom of the same image. Scale bars, 1 mm.

FIG. 3H depicts a violin plot of mCherry intensity, quantified with flow cytometry, dose response to 0, 10, 50, 100, and 200 μg/mL concentrations of microcontact printed GFP after 48 hours. Dotted line indicates the threshold value to designate mCherry-positive cell. Percent of mCherry expressing cells quantified by flow cytometry 48 hours after seeding onto GFP microcontact printed substrate with varying GFP concentrations. Data represents mean±s.d, n=6-8, p<0.05(*)

FIG. 3I depicts a heatmap demonstrating Pearson correlation coefficient between mCherry and GFP signals of each square width and interspace length at Days 2, 5, and 10. Color map represents the mean coefficient, n=7-8.

FIG. 3J depicts microcontact printed 100 μm GFP squares with 100 m interspace length and resulting mCherry activation signals taken two days after seeding. Scale bars, 1 mm.

FIG. 3K depicts fluorescence images of mCherry activation from days 1-12 on 250 and 500 μm width GFP squares with varying interspace lengths.

FIGS. 4A-4D depicts patterned GFP and mCherry spatially activate respective reporter genes via synNotch activation in dual receiver fibroblasts. (4A) Schematic showcasing: (left) dual-ligand microcontact printing, and (right) seeding of dual-receiver L929 cell where anti-GFP synNotch drives miRFP reporter gene and anti-mCherry synNotch orthogonally activates BFP. (4B) Fluorescence microscopy images of microcontact printed GFP and mCherry perpendicular rows of 500 μm width and subsequent dual reporter expression taken 24 hours after uniform seeding. Scale bars, 500 μm. (Right) Normalized plot profiles of miRFP intensity across each row axis of engineered dual-receivers 24 hours following seeding onto perpendicular GFP and mCherry patterns. Green bars indicate regions containing GFP. Line profiles represent mean±s.d, n=7. (Below) Normalized plot profiles of BFP intensity across each row axis of engineered dual-receivers 24 hours following seeding onto perpendicular GFP and mCherry patterns. Red bars indicate regions containing mCherry. Line profiles represent mean±s.d, n=7. Regions of interest on mCherry (red border), on GFP (green border), or on intersecting patterns (yellow border) enlarged in the following panels. Higher magnification fluorescence microscopy images of GFP and mCherry perpendicular rows and subsequent dual reporter expression/brightfield image. Regions of interest on GFP and mCherry intersection (blue border) or non-patterned region (black border) enlarged in the following panels. Scale bars, 500 μm. (4C) Higher magnification fluorescence microscopy images to demonstrate BFP and miRFP expression by dual-receiver fibroblasts on mCherry, GFP, intersection, and between patterns. Scale bars, 100 μm. (4D) Percent reporter activation quantification on the regions containing no ligand, GFP only, mCherry only, and GFP and mCherry. Results show the percent of cells that are not expressing BFP and miRFP, expressing both BFP and miRFP, expressing BFP only, or expressing miRFP only, or n=4.

FIG. 4E depicts a schematic of dual-reporter L929 cell where anti-GFP synNotch drives miRFP reporter gene and anti-mCherry synNotch orthogonally activates BFP.

FIG. 4F depicts percent reporter activation of reporter activation in dual-receiver fibroblasts measured via flow cytometry on no ligand, GFP only, mCherry only, or both GFP and mCherry. Surfaces were uniformly adsorbed with the ligands. Data represents mean s.d, n=4.

FIG. 4G depicts normalized plot profiles of miRFP and BFP intensity across the x-axis 48 hours following seeding onto GFP and mCherry droplet pattern. Line profiles represent mean s.d, n=4.

FIGS. 5A-5J depict microcontact printed GFP patterns spatially activate myoD and initiate myotube differentiation in embryonic fibroblasts via synNotch. (5A) Schematic of embryonic fibroblasts expressing anti-GFP synNotch activating myoD and mCherry transgenes seeded onto GFP patterned PDMS substrate. (5B) Sarcomeric α-actinin staining on isotropic GFP patterns on a PDMS substrate, stained three days following seeding on GFP patterns. (−)GFP indicates image taken on GFP-negative regions, (+)GFP indicates images taken within GFP-positive regions. Scale bars, 1 mm. (5C) Heatmap of hierarchical clustering of fibroblast parental cells without GFP, fibroblasts engineered with anti-GFP synNotch activating myoD and mCherry with and without GFP, and C2C12 myoblasts (skeletal muscle positive control). Z-Score is calculated by (Gene expression value in sample of interest)−(Mean expression across all samples)/Standard Deviation. n=2-4. (5D) Volcano plot of gene expression data showing differentially expressed genes of anti-GFP synNotch activating myoD and mCherry (anti-GFP/myoD) on GFP vs off GFP. n=2-4. (5E) GO term analysis showcasing enriched muscle pathways in myoD expressing synNotch receiver cells on GFP vs off GFP. n=2-4. (5F) Fluorescence microscopy images of anti-GFP/myoD synNotch receiver fibroblasts stained for α-actinin 4 days following seeding onto micromolded gelatin substrates in the presence or absence of GFP. Scale bars, 500 μm. (5G) Myogenic index, quantified with image analysis, in the presence or absence of GFP on isotropic or micromolded gelatin. Data represents mean±s.d, n=5, p<0.0001(****). Myotube alignment of α-actinin stained myotubes on GFP-conjugated micromolded gelatin compared to GFP-conjugated isotropic gelatin substrate, quantified by image analysis. Line plot represents average angles of orientation distribution of 5 individual images from an individual sample. Data represents mean±s.d. (5H) Binary mask used to generate stamps for microcontact printing followed by fluorescence microscopy images of GFP ligand and subsequent α-actinin staining for each pattern: 500 μm curves, 200 μm curves, 500 μm rows, and 200 μm rows. Samples were stained three days following uniform seeding onto GFP patterns. (5I) Day 3 Myogenic Index, quantified with image analysis, on and off GFP for each pattern (isotropic, 200, 500 curves, 200, 500 straight rows). Data represents mean±s.d, n=3-6, p<0.01(**), p<0.001(***), p<0.0001(****). (5J) Orientation Order Parameter measured across all patterns quantified with image analysis. Significance values compared to isotropic control. Data represents mean±s.d, n=3-8, p<0.05(*), p<0.01(**).

FIG. 5K depicts a volcano plot of gene expression data showing differentially expressed genes of anti-GFP synNotch mCherry reporter fibroblasts on GFP vs off GFP. n=2.

FIG. 5L depicts nuclei alignment quantifications of anti-GFP synNotch MyoD fibroblasts in the presence or absence of GFP on isotropic or micromolded gelatin. Data represents mean±s.d, n=5, p<0.0001(****).

FIG. 5M depicts plot profiles of normalized α-actinin expression across 500 and 200 μm rows on non-restricted GFP patterns. Green lines indicate the region containing GFP.

FIGS. 6A-6G depict mCherry activates ETV2 and reporter BFP to induce endothelial differentiation in embryonic fibroblasts via synNotch. (6A) Schematic of embryonic fibroblasts cell line (C3H) expressing anti-mCherry/Gal4 synNotch activating ETV2 and BFP transgenes seeded onto mCherry patterned substrate. (6B) Fluorescence microscopy images of BFP reporter (top), endothelial markers VEGFR2 (middle) and VE-Cadherin (bottom) stained 3 days following seeding onto control wells (−mCherry) or plate-dried mCherry (+mCherry). Scale bars, 200 μm. (6C) Percent of cells expressing BFP (left) and VEGFR2 (right) in the presence (+mCherry) or absence (−mCherry) of mCherry, quantified with flow cytometry. Data represents mean±s.d, BFP n=12-13, VEGFR2 n=4-5, p<0.001(***), p<0.0001(****). (6D) Heatmap of hierarchical clustering of fibroblast parental cells without mCherry, fibroblasts engineered with anti-mCherry synNotch activating ETV2 and BFP with and without mCherry, and BEnd.3 endothelial cells (positive control). (6E) Volcano plot of gene expression showing differentially expressed genes of anti-mCherry synNotch cells activating ETV2 and BFP on mCherry vs off mCherry, n=2. (6F) Binary mask used to generate stamps for microcontact printing of 500 μm rows followed by fluorescence microscopy images of day 3 nuclei staining, BFP expression, and VEGFR2 immunostaining. Scale bars are 1 mm. Plot profile of normalized BFP and VEGFR2 intensity on Day 3 following seeding onto 500 μm mCherry rows. Red bars indicate the regions containing mCherry. Line profiles represent mean±s.d, n=2. (6G) Binary mask used to generate stamps of vasculature-like pattern followed by fluorescence microscopy images of day 3 nuclei staining, BFP expression, and VEGFR2 immunostaining.

FIG. 6H depicts normalized BFP intensity of anti-mCherry synNotch fibroblasts seeded on varying concentrations of plate-dried mCherry up to 3 days. Data represents mean±s.d, n=3.

FIG. 6I depicts measurement of CDH5 (VE-Cadherin) via immunostaining and flow cytometry with and without presence of mCherry. Data represents mean±s.d, n=3-4, p<0.01(**).

FIG. 6J depicts Principal Component Analysis (PCA) comparing the transcriptome of unmodified C3H fibroblasts, cell type-specific positive control cells (C2C12 and Bend.3), and receiver cells that expressing mCherry, MyoD and mCherry, or ETV2 and BFP in the presence or absence of their corresponding ligand. N=2-4.

FIGS. 7A-7F depict spatially controlled co-transdifferentiation into myogenic and endothelial lineages of dual-lineage synNotch-engineered cells culturing on micropatterned ligands. (7A) Schematic showing dual protein patterning technique using capillary-driven microfluidic patterning, based on shallow and deep channels, to generate parallel lines of GFP and mCherry. Feature size, 500 μm. Schematic of dual-lineage mouse embryonic fibroblasts (C3H line) expressing anti-GFP/tTA synNotch that activates MyoD and miRFP as well as an anti-mCherry/Gal4 synNotch that orthogonally activates ETV2 and BFP transgenes, seeded onto GFP and mCherry patterned substrate. (7B) Fluorescence images of GFP and mCherry ligand (left), reporter genes expression (center) and brightfield (right) of dual-lineage cells 3 days following uniform seeding onto micropatterned ligands. Dotted white rectangles represent regions of interest for quantification. Scale bars, 1 mm. Plot profiles on the right show normalized fluorescence intensity of ligands and reporters taken in the region of interest. Green and red bars indicate regions containing GFP or mCherry, respectively. (7C) Center: merged fluorescence image of α-actinin and VEGFR2 immunostaining on dual-lineage cells uniformly seeded onto GFP and mCherry pattern. Dotted green lines represent region where the GFP signal has been subtracted to visualize VEGFR2 staining. Scale bar, 1 mm. Around the central image, higher magnification fluorescence images taken within distinct regions of the pattern are shown: on mCherry (red border), interface between mCherry and GFP regions (yellow border), on GFP (green border), and off pattern (beige border). Scale bars, 200 μm. Higher magnification of interface (blue border), visualizing α-actinin and VEGFR2 staining is shown on the far right with a scale bar of 50 μm. (7D) Schematic showing the different ligand patterns used in single-nuclei sequencing experiments. T-Distributed Stochastic Neighbor Embedding plot results of dual-lineage fibroblasts cultured on the four different patterning conditions. Fibroblast-like cluster contains seven individual clusters, shown here as one beige color. n=2. (7E) Percent of Fibroblast-like, muscle-like, and endothelial-like cells across the four patterning conditions (left). Percent of each muscle-like cluster (of the total muscle-like cells) in each patterning condition (right). n=2. (7F) Plot showing average expression and percent expression of selected fibroblast, muscle, and endothelial markers in all clusters across different patterning conditions. n=2.

FIG. 7G depicts normalized BFP intensity of dual-lineage synNotch fibroblasts seeded on varying concentrations of plate-dried mCherry up to 3 days. Data represents mean±s.d, n=3.

FIG. 7H depicts dual-lineage receiver cells cultured on GFP only, mCherry only, or both GFP and mCherry. Surfaces were uniformly adsorbed with the ligands.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

“Operably linked” refers to a juxtaposition wherein the components so described are in a relationship permitting them to function in their intended manner. For instance, a promoter is operably linked to a coding sequence if the promoter affects its transcription or expression.

“Heterologous,” as used herein, means a nucleotide or polypeptide sequence that is not found in the native (e.g., naturally-occurring) nucleic acid or protein, respectively.

The term “ligands” include polypeptides, nucleic acids, glycoproteins, small molecules, carbohydrates, lipids, glycolipids, lipoproteins, lipopolysaccharides, etc. In some cases, the ligand is soluble. In preferable embodiments, a ligand is a biologically inert polypeptide, e.g., such as recombinant or synthetic fluorescent polypeptides that is not naturally produced by and/or would not detectably affect biological activity of a mammalian cell.

The term “binding” refers to a direct association between two molecules, due to, for example, covalent, electrostatic, hydrophobic, and ionic and/or hydrogen-bond interactions, including interactions such as salt bridges and water bridges. In some cases, a ligand-specific targeting region present in the extracellular domain of a synthetic Notch receptor of the present disclosure binds specifically to a ligand or a fusion protein comprising the ligand and another polypeptide or a cell presenting the ligand. Typically “specific binding” refers to binding with an affinity of at least about 10⁻⁷ M or greater, e.g., 5×10⁻⁷ M, 10⁻⁸M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

In some cases, a naturally-occurring Notch receptor polypeptide includes: a) an extracellular portion that includes: i) epidermal growth factor (EGF) repeats; ii) a ligand binding site; iii) three Lin-12 Notch repeats (LNR), designated LNR-A, LNR-B, and LNR-C; iv) two heterodimerization domains (HD-N and HD-C); b) a transmembrane (TM) portion; and c) an intracellular portion that includes: i) a RAM domain; ii) ankyrin repeats; iii) a transcription activation domain; and iv) a PEST region.

In some cases, a Notch receptor regulatory region comprises Lin-12 Notch repeats A-C, heterodimerization domains HD-N and HD-C, a binding-induced proteolytic cleavage site, and a transmembrane domain. In some cases, a binding-induced proteolytic cleavage site is an S2 proteolytic cleavage site or a S3 proteolytic cleavage site. A synNotch receptor includes three proteolytic sites, termed S1, S2, and S3. S1, a furin cleavage site, is located between HD-N and HC-C; S2, an ADAM17 cleavage site, is located within HD-C; and S3, a gamma secretase cleavage site, is within the TM portion.

“Antigen-specific targeting region” (ASTR) as used herein refers to the extracellular region of a synthetic receptor (e.g., synthetic Notch receptor) which targets specific ligands; and hence may also be called “ligand-specific targeting region”. The targeting regions may comprise full length heavy chain, Fab fragments, single chain Fv (scFv) fragments, divalent single chain antibodies or diabodies, each of which are specific to the target ligands. In some embodiments, the ASTR is a nanobody. In some embodiments, the ASTR is a single-domain antibody. In some embodiments, the ASTR is a diabody. In some embodiments, the ASTR is a triabody. In some embodiments, the ASTR is a minibody. In fact, almost any molecule that binds a given ligand with high affinity and selectivity can be used as an antigen-specific targeting region, as will be appreciated by those of skill in the art. Hence, in some embodiments, it is referred to as a first member of a specific binding pair. That is, an extracellular domain of a synNotch receptor comprises a first member of a binding pair.

The term “linker” with respect to amino acid linker in a polypeptide can be a short peptide. Examples of these short peptides include but are not limited to a dimer of two amino acids, a tri-mer of three amino acids, a 4-mer of four amino acids, a 5-mer of five amino acids, or a peptide selected from the group consisting of T, PT, MPT, S, GS, GGS, GGGS (SEQ ID NO:9), and (GGGGX_{λ} (SEQ ID NO:10))n wherein X_{λ} is Q, A, E or S and n=1-5 or an integer larger than 5. In some embodiments, the amino acid linker has the amino acid sequence of (GGGGS (SEQ ID NO:11))n where n is an integer between 1 and 5, thereby an amino acid linker of 25 amino acids or shorter in length.

In various instances, a synNotch receptor refers to a polypeptide comprising, from N-terminal to C-terminal and in covalent linkage: a) an extracellular domain comprising a first member of a specific binding pair; b) a Notch receptor polypeptide, wherein the Notch receptor polypeptide has a length of from 50 amino acids to 1000 amino acids, and comprises one or more ligand-inducible proteolytic cleavage sites (e.g., an S2 proteolytic cleavage site or a S3 proteolytic cleavage site); and c) an intracellular domain, wherein the first member of the specific binding pair is heterologous to the Notch receptor polypeptide, and wherein binding of the first member of the specific binding pair to a second member of the specific binding pair induces cleavage of the Notch receptor polypeptide at the one or more ligand-inducible proteolytic cleavage sites, thereby releasing the intracellular domain. In some cases, a synNotch receptor comprises a linker interposed between the extracellular domain and the Notch receptor polypeptide. Amino acid sequences of exemplary synNotch receptors are described in U.S. Pat. No. 10,590,182, which is incorporated herein by reference in its entirety.

In some cases, the intracellular domain of a synNotch receptor is a transcriptional activator. In some cases, the intracellular domain is a transcriptional repressor. In other cases, the intracellular domain is a site-specific nuclease, a recombinase, an inhibitory immunoreceptor, or an activating immunoreceptor. In some cases, the intracellular domain is a transcription factor. Examples of suitable transcription factors are those presented in Table 1 of U.S. Patent Application No. 2014/0308746, which is incorporated by reference.

Synthetic Notch (synNotch) receptors are modular synthetic components that are genetically engineered in mammalian cells to detect signals presented by neighboring cells and respond by activating prescribed transcriptional programs. synNotch is used to program therapeutic cells and pattern morphogenesis in multicellular systems. SynNotch endow cells with orthogonal, customizable signaling capacities, and so can be used to control spatial gene expression patterns in engineered tissues. However, cell-presented ligands have limited versatility for applications that require spatial precision, such as tissue engineering. Furthermore, whether material-presented ligands can provide spatial patterning of ligands at different resolution and across different biomaterials with extracellular matrix manipulations has not been demonstrated so far.

To address this, we herein present a suite of materials to activate synNotch receptors and serve as generalizable platforms for generating user-defined material-to-cell signaling pathways. First, we demonstrate that synNotch ligands, such as green fluorescent protein (GFP), can be conjugated or fused to cell-generated ECM proteins, e.g., via genetic engineering of fibronectin-GFP produced by fibroblasts. We then used enzymatic or click chemistry to covalently link synNotch ligands to gelatin polymers to activate synNotch receptors in cells grown on or within a hydrogel. To achieve microscale control over synNotch activation in cell monolayers, we herein present microcontact printing synNotch ligands onto a surface and demonstrate orthogonal control of intracellular gene activations with two types of synNotch ligands and four distinct reporter gene profiles. We also patterned tissues comprising cells with up to three distinct phenotypes by engineering cells with two distinct synthetic pathways and culturing them on surfaces microfluidically patterned with two synNotch ligands. We showcase this technology by co-transdifferentiating fibroblasts into skeletal muscle or endothelial cell precursors in user-defined spatial patterns towards the engineering of muscle tissue with prescribed vascular networks. Collectively, this suite of approaches extends the synNotch toolkit and provide new avenues for spatially controlling cellular phenotypes in mammalian multicellular systems, with many broad applications in developmental biology, synthetic morphogenesis, human tissue modeling, and regenerative medicine.

We reasoned that the integration of synthetic signaling pathways, where both the input and output are user-defined, can serve as a useful tool for control of spatial gene expression in engineered tissues. We have conceived that these receptors could be leveraged to spatially control gene expression patterns in engineered tissues with more precision than endogenous receptors. We have turned to synNotch pathways that allow for specific user-defined outputs in response to non-native synthetic ligands. In this case, the user can define which ligand is the input ligand and what output gene is activated once a cell is stimulated with the cognate ligand. To achieve this modularity, synNotch receptors are a derivative of Notch receptors in which the extracellular sensor module and intracellular transcriptional module are replaced with user-defined heterologous protein domains. In the currently deployed version, a neighboring cell is producing a synNotch ligand (for example membrane-tethered GFP); engagement of the synNotch receptor with its ligand leads to intramembrane proteolysis which releases the intracellular fragment of synNotch which is a transcriptional factor that is orthogonal to mammalian signaling. SynNotch receptors can be used to induce differentiation of mammalian cells by non-native ligands when outputs of the synNotch pathway are master transcription factors that induce differentiation when overexpressed. The synNotch technology is modular and allows for generation of multi-input logic in cells: cells can be engineered to express 2 different gene cassettes upon stimulation with 2 different synthetic inputs. In this configuration, it has been shown to generate a limited numbers of spatial patterns of gene expression in 2D (concentric rings) and in 3D (polarized and layered spheroids) by using neighboring cells (i.e., sender cells) to present synthetic ligands to cells expressing synNotch (i.e., receiver cells). However, with cellular ligand presentation, controlling the geometry of synthetic ligands necessitates controlling the location of sender cells, making the problem circular.

Additionally, synNotch receptor can be activated by ligands presented from surfaces other than cells. That is, synNotch ligands can also be active when presented from different materials (for example DNA-scaffolds can activate, adsorbed on a surface and also to pull on it to expose the proteolytic domain). It has been shown that synthetic ligands such as GFP can successfully activate synNotch circuits when passively adsorbed onto cell growth surfaces, tethered by DNA linkers to microbeads, and attached to atomic force microscopy. More recently, an approach to specifically activate synNotch from culture surfaces was developed under the acronym MATRIX. These approaches have not been used for patterning gene expression and/or for differentiation and co-differentiation of multiple cell fates within the same culture with micron scale precision.

Here we conceived and demonstrated that the synNotch receptors can be used as a platform to generate synthetic signaling pathways from a material to a cell to spatially control gene expression and differentiation. In these approaches, mouse fibroblast cells are genetically engineered with synthetic receptors of the synNotch family, and their cognate ligands are presented from a material. We developed at least 3 ways to present ligands to activate synNotch pathways: (1) genetically encoded, cell-produced ECM proteins (e.g., fibronectin-GFP fusions); (2) microcontact printed culture surfaces with ligands laid down on cell culture ready surfaces (e.g., 2D spatial patterns of one, and then two, synthetic signals (e.g., GFP and mCherry)); (3) ECM-derived hydrogel (e.g., hydrogel engineered with click chemistry to conjugate signals in 3D). These ligands can be patterned in space to various degrees of precision. We show spatial activation with for example 100s μm precision via microcontact-printing of one or two ligands. We then show application of spatial control of gene expression in trans-differentiation assays where embryonic fibroblasts are differentiated into either skeletal muscle precursors or endothelial cell precursors in spatially controlled manner, eventually coming from a single dual-fate engineered cell population. Furthermore, we demonstrate herein methods for spatially controlling the co-transdifferentiation of somatic cells (e.g., fibroblasts) to one of two cell fates (e.g., endothelial precursor cells or even functional endothelial cells, and skeletal muscle precursors) in a continuous tissue construct. This was achieved by generating dual-lineage fibroblasts expressing two independent synNotch receptors (one for endothelial transdifferentiation, and one for muscle transdifferentiation) and culturing these cells on a surface with the two synthetic cognate ligands patterned via a microfluidic device.

Cells engineered with anti-GFP synNotch pathways that activate reporter genes are then cultivated on the material. We show their activation where the ligands were presented. With this setup, we first show the proof-of-principle of the different technologies, and then we focus on one of those, microcontact printing of 2 synthetic ligands (GFP and mCherry), to define its capacity and limitations of spatially and temporally activating two independent reporter genes in user-defined spatial patterns. We then show that we can control differentiation from fibroblasts to either myoblasts or endothelial cell precursors via growing the cells with the synthetic pathway on a scaffold that presents the ligands. Moreover, we show that we can control differentiation in space with a high resolution. Finally, we use this setup to generate constructs where fibroblasts, endothelial precursors and skeletal muscle precursor are all differentiated concurrently from multipotent precursor cells in user-defined arrangements that mimic skeletal muscle cito-architecture. This is important because it is a technology that is potentially modular, and can be used with other transcription factors in a compositionable manner. This technology could pave the way for more controlled differentiation of in vitro grown tissue and organs at the cellular scale.

synNotch-mediated generation of spatial patterns of gene expression adds a powerful and flexible functionality to the multicellular synthetic biology approaches for the study and the control of multicellular organization.

In various embodiments, synNotch is composed of a chimeric protein from N-to-C terminus: an antibody-based binding domain or antigen-specific targeting domain, as the extracellular domain (e.g., anti-GFP nanobody, anti-mCherry nanobody); a Notch regulatory region (e.g., the Notch juxtamembrane and transmembrane domains); and an orthogonal transcription factor, as the intracellular domain. In some embodiments, one or more linkers or spacers are also present in synNotch, such as between the antigen-specific targeting domain and the Notch regulatory region, and/or between the Notch regulatory region and the transcription factor. In some embodiments, a linker or spacer is a peptide between 3-30 amino acids long. In some embodiments, a linker is a peptide having an amino acid sequence of SEQ ID NO:7 or 8.

SynNotch has many desirable features such as: (i) the receptor is not activated by soluble factors, as it requires a pulling force to induce the conformational change necessary for activation; (ii) the ligand is customizable and can be an orthogonal inert molecule, such as green fluorescent protein (GFP) or mCherry; (iii) receptor activation can drive customizable cellular responses, such as differentiation, when combined with complementary genetically engineered cassettes.

In some embodiments, the extracellular domain of a synNotch receptor targeting a ligand (e.g., GFP) comprises a nanobody (e.g., a GFP nanobody), or “LaG” (Llama antibody against GFP)—a nomenclature according to Fridy et al. (2014) Nat. Methods. 11(12):1253-1260. In instances where GFP (or mutant of GFP or other Cnidarian fluorescent proteins related to GFP (e.g., AmCFP, DsRed, etc.)), is used as the ligand, various combinations of LaG nanobodies may find use provided the members of the LaG nanobody pair do not interfere with one another in their binding to GFP, e.g., where the members of the pair of LaG nanobodies bind different epitopes of GFP. LaG nanobodies include but are not limited to e.g., LaG-2, LaG-3, LaG-6, LaG-9, LaG-10, LaG-12, LaG-14, LaG-16, LaG-17, LaG-19, LaG-21, LaG-24, LaG-26, LaG-27, LaG-29, LaG-30, LaG-35, LaG-37, LaG-41, LaG-42, LaG-43, LaG-5, LaG-8, LaG-11, LaG-18, LaG16-G4S-2, LaG16-3×FLAG-2, LaG41-G4S-2, and the like.

In some embodiments, the extracellular domain of a synNotch receptor targeting mCherry comprises llama antibodies against mCherry (LaM), e.g., LaM nanobodies include but are not limited to e.g., LaM-1, LaM-2, LaM-3, LaM-4, LaM-6, LaM-8.

In some embodiments, the intracellular domain is a transcriptional activator. In some cases, the intracellular domain comprises an amino acid sequence having at least 75%, at least 80%, at least 85%, at least 90%, at least 95%, at least 98%, at least 99%, or 100%, amino acid sequence identity to tetracycline-controlled transcriptional activator (tTA, also called tetracycline transactivator, a regulator of transcription activation). In some cases, the intracellular domain comprises an amino acid sequence having at least 75%, at least 80%, at least 85%, at least 90%, at least 95%, at least 98%, at least 99%, or 100%, amino acid sequence identity to GAL4-VP16. In some cases, the intracellular domain comprises an amino acid sequence having at least 75%, at least 80%, at least 85%, at least 90%, at least 95%, at least 98%, at least 99%, or 100%, amino acid sequence identity to GAL4-VP64. In some embodiments, the intracellular domain comprises an amino acid sequence having at least 75%, at least 80%, at least 85%, at least 90%, at least 95%, at least 98%, at least 99%, or 100%, amino acid sequence identity to MyoD.

In various embodiments, cells to be modulated are genetically engineered with a synthetic receptor (or engineered receptor), wherein the synthetic receptor (or engineered receptor) has an extracellular domain capable of binding a specific ligand and an intracellular domain, such that upon binding of the specific ligands from outside of the cell, the cells are driven for desired modulation (such as transdifferentiation). Exemplary synthetic receptors include but are not limited to receptors activated solely by synthetic ligands (RASSL, where the ligand binding domain has been engineered to respond to a pharmacologically inert small molecule drug), chimeric antigen receptor (comprising an extracellular antigen-binding domain, transmembrane domain, and intracellular co-stimulatory domains), SynNotch receptor, modular extracellular sensor architecture (MESA, which senses extracellular ligands without reliance on native receptors or signaling pathways), and Tango receptors (wherein a membrane protein is fused to a transcription factor with a TEV protease binding site linker). Further description is provided in Mendeley et al., Current Opinion in Systems Biology, Volume 28, December 2021, 100363.

In various embodiments, a synthetic receptor is a synthetic Notch receptor, which comprises an extracellular domain that specifically binds an antigen and an intracellular domain that comprises a transcriptional activator, wherein binding of the extracellular domain to the antigen (particularly in trans, i.e., from a different cell) induces cleavage in the synthetic Notch receptor, thereby releasing the intracellular domain, causing the transcriptional activator to induce expression of an endogenous or heterologous gene product in a cell expressing the synthetic Notch receptor.

In various embodiments, an extracellular domain that specifically binds an antigen is also referred to as an antigen-specific targeting domain. In some embodiments, the antigen-specific targeting domain comprises or consists of a single-chain Fv (scFv). In some embodiments, the antigen-specific targeting domain comprises of consists of a nanobody that specifically binds to the antigen.

A polypeptide region/domain heterologous to a Notch receptor polypeptide means that the member is not naturally present in a Notch receptor polypeptide. In some embodiments, a synthetic Notch receptor is also referred to as a chimeric Notch polypeptide. Further description of synthetic Notch receptors and exemplary orthogonal binding ligands is provided in U.S. Pat. No. 9,670,281, which is incorporated by reference in its entirety.

Various embodiments provide one or more combinations or systems, which include: (i) a ligand, positioned in a predetermined pattern on/in a substrate, (ii) a cell genetically engineered to express a synthetic receptor (e.g., synNotch receptor), and (iii) the substrate. Optionally a system or combination further includes (iv) a culture device (e.g., a culture surface), where the cell expressing the synthetic receptor is cultured. Preferably the ligand is an inert molecule that does not detectably alter the viability of a cell in the system. For example, the viability of a cell when in contact/exposure to the ligand remains at least 100%, 95%, 90%, 85%, or 80% compared to that in the absence of the ligand; and/or the activity of a cell (with no expression of an anti-ligand synNotch receptor) when in contact/exposure to the ligand remains at least 100%, 95%, 90%, 85%, or 80% compared to that in the absence of the ligand.

Some embodiments provide one or more combinations or systems, which include: (i) a fusion protein, positioned in a predetermined pattern on/in a substrate, wherein the fusion protein comprises a ligand and another molecule (e.g., the other molecule may be an extracellular matrix molecule, such as polypeptide or proteoglycan; or a fragment crystallizable region (Fc); or a polymer such as poly(ethylene glycol) or poly-L-lysine, poly-D-lysine, poly-L-ornithine; or a combination thereof), (ii) a cell genetically engineered to express a synthetic receptor (e.g., synNotch receptor), and (iii) the substrate. Optionally a system or combination further includes (iv) a culture device (e.g., a culture surface), where the cell expressing the synthetic receptor is cultured. In some aspects, an extracellular matrix molecule may be a full-length or a fragment of an extracellular protein or proteoglycan, such as fibronectin, gelatin, laminin, collagen, vitronectin, hyaluronic acid, heparan sulphate proteoglycan.

Some embodiments provide one or more combinations or systems, which include: (i) a first cell, positioned in a predetermined pattern on/in a substrate, wherein the first cell secrete or express on its cell surface a ligand or a fusion protein comprising the ligand and an extracellular matrix polypeptide, (ii) a second cell genetically engineered to express a synthetic receptor (e.g., synNotch receptor), and (iii) the substrate. Optionally a system or combination further includes (iv) a culture device (e.g., a culture surface), where the cell expressing the synthetic receptor is cultured. In some aspects, the first cell and the second cell are different types of cells. In other aspects, the first cell and the second cells are of a same type of cells.

Some embodiments provide one or more combinations or systems, which include: (i) two or more of: a ligand, a fusion protein comprising the ligand and a macromolecule, and a first cell secreting or expressing on its cell surface the ligand or a fusion protein comprising the ligand and an extracellular matrix polypeptide, positioned in a predetermined pattern on/in a substrate (ii) a second cell genetically engineered to express a synthetic receptor (e.g., synNotch receptor), and (iii) the substrate. Optionally a system or combination further includes (iv) a culture device (e.g., a culture surface), where the cell expressing the synthetic receptor is cultured.

In various aspects, the ligand, the fusion protein, or the cell, are covalently or non-covalently bonded to a substrate in a micropattern. In various aspects, the ligand, the fusion protein, or the cell, are photocrosslinked to a substrate in a micropattern. Typical functional groups for conjugation (e.g., crosslinking, click chemistry) and ways to conjugate functional groups onto protein molecules are known to those skilled in the art and described in literature such as Bioconjugate Techniques, 2nd Edition, by Greg T. Hermanson. In various aspects, the bonded ligand/fusion protein/cell to the substrate can bind to and activate a synNotch receptor at an efficiency that is at least 95% of that when the ligand/fusion protein/cell is in a free form or does not bind to the substrate. In various aspects, the bonded ligand/fusion protein/cell to the substrate can bind to and activate a synNotch receptor at an efficiency that is at least 90% of that when the ligand/fusion protein/cell is in a free form or does not bind to the substrate. In various aspects, the bonded ligand/fusion protein/cell to the substrate can bind to and activate a synNotch receptor at an efficiency that is at least 85% of that when the ligand/fusion protein/cell is in a free form or does not bind to the substrate. In various aspects, the bonded ligand/fusion protein/cell to the substrate can bind to and activate a synNotch receptor at an efficiency that is at least 80% of that when the ligand/fusion protein/cell is in a free form or does not bind to the substrate. In various aspects, the bonded ligand/fusion protein/cell to the substrate can bind to and activate a synNotch receptor at an efficiency that is at least 75% of that when the ligand/fusion protein/cell is in a free form or does not bind to the substrate. In various aspects, the bonded ligand/fusion protein/cell to the substrate can bind to and activate a synNotch receptor at an efficiency that is at least 70% of that when the ligand/fusion protein/cell is in a free form or does not bind to the substrate. In various aspects, the bonded ligand/fusion protein/cell to the substrate can bind to and activate a synNotch receptor at an efficiency that is at least 60% of that when the ligand/fusion protein/cell is in a free form or does not bind to the substrate. In various aspects, the bonded ligand/fusion protein/cell to the substrate can bind to and activate a synNotch receptor at an efficiency that is at least 50% of that when the ligand/fusion protein/cell is in a free form or does not bind to the substrate.

In various aspects, a substrate is an elastomeric stamp (e.g., PDMS stamp). In some aspects, a substrate is a microparticle. In some aspects, a substrate is a polymeric scaffold or hydrogel. In some aspects, a substrate is a coverslip. In some aspects, a substrate is a microfluidic device. In some aspects, a substrate is a coverslip having micropatterns of the ligands deposited via a elastomeric stamp or a microfluidic device.

In various aspects, a pattern comprises a plurality of features. In some aspects, a pattern comprises a plurality of repetitive features. In various aspects, a pattern is a micropattern comprising a plurality of ligand-distributed, micron sized features. In some aspects, a feature is in a dimension of 50-1000 μm. For example, a feature is in a dimension of 50-100 μm. For example, a feature is in a dimension of 100-200 μm. For example, a feature is in a dimension of 200-300 μm. For example, a feature is in a dimension of 300-400 μm. For example, a feature is in a dimension of 400-500 μm. For example, a feature is in a dimension of 500-600 μm. For example, a feature is in a dimension of 600-700 μm. For example, a feature is in a dimension of 700-800 μm. For example, a feature is in a dimension of 800-900 μm. For example, a feature is in a dimension of 900-1000 μm. In various aspects, the spacing/distance between two neighbouring features is in the micron size, e.g., between 50-100, 100-200, 200-300, 300-400, 400-500, 500-600, 600-700, 700-800, 800-900, or 900-1000 μm.

In some aspects, a micropattern has non-overlapping features. In other aspects, a micropattern has features that are overlapping.

In some embodiments, a substrate is a poly(dimethylsiloxane) (PDMS) stamp having a pattern with a plurality of features, wherein an interspace between two neighboring features being 200-250 μm. In some embodiments, a substrate is a poly(dimethylsiloxane) (PDMS) stamp having a pattern with a plurality of features, wherein an interspace between two neighboring features being 250-300 μm. In some embodiments, a substrate is a poly(dimethylsiloxane) (PDMS) stamp having a pattern with a plurality of features, wherein an interspace between two neighboring features being 300-350 μm. In some embodiments, a substrate is a poly(dimethylsiloxane) (PDMS) stamp having a pattern with a plurality of features, wherein an interspace between two neighboring features being 350-400 μm. In some embodiments, a substrate is a poly(dimethylsiloxane) (PDMS) stamp having a pattern with a plurality of features, wherein an interspace between two neighboring features being or 400-500 μm.

In some embodiments, a method of co-differentiation or co-transdifferentiation of a cell includes: contacting the cell with a first ligand, a first fusion protein comprising the first ligand and an extracellular matrix polypeptide, or a first sender cell secreting or expressing on cell surface the first ligand, and with a second ligand, a second fusion protein comprising the second ligand and an extracellular matrix polypeptide, or a second sender cell secreting or expressing on cell surface the second ligand, wherein the cell expresses: i) a first synNotch receptor comprising, in N-terminal to C-terminal order: an extracellular domain comprising a targeting region specifically targeting the first ligand, wherein the first ligand-targeting region is preferably heterologous to the Notch receptor; a Notch receptor regulatory region; and a first intracellular domain preferably heterologous to the Notch receptor; and ii) at least a second synthetic Notch receptor comprising, in N-terminal to C-terminal order: an extracellular domain comprising a targeting region specifically targeting the second ligand, wherein the second ligand-targeting region is preferably heterologous to the Notch receptor; a Notch receptor regulatory region; and a second intracellular domain preferably heterologous to the Notch receptor; wherein the first intracellular domain, when cleaved from the Notch receptor regulatory region or ‘activated’, provides a first effector function, and the second intracellular domain, when cleaved from the Notch receptor regulatory region or ‘activated’, provides a second effector function that is different from the first effector function, wherein the first and the second effector functions modulates differentiation of the cell (preferably into different types of cell). In further embodiments, a Notch receptor regulatory domain comprises Lin-12 Notch repeats A-C, heterodimerization domains HD-N and HD-C, a binding-induced proteolytic cleavage site, and a transmembrane domain. In some embodiments, a Notch receptor regulatory region comprises a polypeptide having an amino acid sequence of SEQ ID NO:5.

In preferably embodiments, the method of co-differentiation or co-transdifferentiation of a cell includes co-differentiation or co-transdifferentiation of a cell in a predetermined pattern, wherein the first ligand and the second ligand are provided in the predetermined pattern having a feature size in the micron scale. In some embodiments, a predetermined pattern has a feature size of 1-10 μm. In some embodiments, a predetermined pattern has a feature size of 10-100 μm. In some embodiments, a predetermined pattern has a feature size of 100-200 μm. In some embodiments, a predetermined pattern has a feature size of 200-300 μm. In some embodiments, a predetermined pattern has a feature size of 300-400 μm. In some embodiments, a predetermined pattern has a feature size of 400-500 μm. In some embodiments, a predetermined pattern has a feature size of 500-600 μm. In some embodiments, a predetermined pattern has a feature size of 600-700 μm. In some embodiments, a predetermined pattern has a feature size of 700-800 μm. In some embodiments, a predetermined pattern has a feature size of 800-900 μm. In some embodiments, a predetermined pattern has a feature size of 900-1000 μm. A feature size may relate to the size of a discrete distribution of the ligand(s); or a feature size may also relate to the spacing between regions where ligand(s) are distributed, hence may also be called a featureless region. In some embodiments, a predetermined pattern has a feature size of ligand distribution between 100 μm and 500 μm, the pattern having a plurality of ligand-distributed features, and a spacing/featureless region between two nearest features being between 100 μm and 500 μm.

In further embodiments, the cell (to be differentiated or transdifferentiated) is genetically modified with a nucleic acid comprising a nucleotide sequence encoding a synNotch receptor, and wherein the intracellular domain of the synNotch receptor is a transcriptional activator. In some cases, the nucleotide sequence encoding the synNotch receptor is operably linked to a transcriptional control element (such as a transcription factor) that is activated by the intracellular domain of the synNotch receptor.

In some cases, the cell is a stem cell and the activated intracellular domain of the synNotch effectuates differentiation of the stem cell. For example, the cell may be mesenchymal stem cells, or induced pluripotent stem cells. In some cases, the cell is a progenitor or precursor cell and the activated intracellular domain of the synNotch effectuates differentiation of the progenitor or precursor cell. In some cases, the cell is a somatic cell and the activated intracellular domain of the synNotch effectuates transdifferentiation of the somatic cell. In some cases, the somatic cell comprises fibroblasts.

In some cases, the contacting is carried out in vivo. In some cases, the contacting is carried out ex vivo. In some cases, the contacting is carried out in vitro.

In some embodiments, three-dimensional patterning of a ligand in a substrate includes the steps of: infusing the substrate (e.g., a polymer gel material) with at least one reactive group to form reactive group sites or modifying the substrate with reactive group sites; illuminating selected voxels within the substrate to yield a three-dimensional pattern of reactive group sites anchored to the substrate; and depositing functional molecules (e.g., a ligand, a fusion protein, or a cell secreting or expressing the ligand or fusion protein) on the reactive group sites. In some embodiment, the method of three-dimensional patterning of the ligand in the substrate further includes removing excess reactive groups from the substrate, preferably before depositing the functional molecules. Further description of three-dimensional patterning a polymeric matrix is described in US20170081489, which is incorporated by reference in its entirety.

Methods and reagents for two-dimensional patterning a substrate, such as patterning on a substrate surface of a ligand or a fusion protein comprising the ligand of the invention, may be achieved via microcontact printing, are described in U.S. Pat. No. 7,117,790, which is incorporated by reference in its entirety.

For example, soft lithography can be used for two-dimensionally patterning a substrate, and it is based on a soft elastomeric stamp with patterned relief (i.e., elevation or unevenness of a surface; e.g., having ridges, islands etc. of a height feature difference from bottom). The benefit of soft lithography is that the photolithographic process is only necessary for the fabrication of a master, and multiple copies of elastomeric stamps can be prepared by replica moulding against the master. Because of this, the use of a clean-room environment is minimised. The two major soft-lithographic methods for surface patterning are microcontact printing and microfluidic patterning.

In some embodiments, a predetermined pattern in one or more systems or combinations disclosed herein is produced by microcontact printing, and/or the one or more methods disclosed herein for providing a ligand/fusion protein/sender cell in a predetermined pattern to a substrate is performed by microcontact printing. Preferably, the ligand/fusion protein/sender cell once provided on surface of the substrate is placed in contact with target cells (cells expressing synNotch receptor(s)) within 3 days from microcontact printed on the substrate. In some embodiments, the substrate is placed in contact with target cells (cells expressing synNotch receptor(s)) within 1-5 days from microcontact printing the ligand/fusion protein/sender cell onto the substrate. In some embodiments, the substrate is placed in contact with target cells (cells expressing synNotch receptor(s)) beginning within the first 12 hours from microcontact printing the ligand/fusion protein/sender cell onto the substrate, and lasting for a duration of from 1 minute to 12 hours, 1 day, 2 days, 3 days, 4 days, 5 days.

In various aspects, a ligand or fusion protein is transferred from an elastomeric stamp that has a patterned relief to a substrate at the area contacted by the stamp. For example, by casting a solution of poly(dimethylsiloxane) (PDMS) prepolymer onto a photolithographically fabricated patterned master, a PDMS stamp is replica moulded. This stamp is immersed into a solution of the ligand or fusion protein of interest and then stamped to a substrate via conformal contact to the surface, thereby transferring the material to the substrate.

In some implementations, a PDMS stamp with predetermined features is immersed in a ligand solution, resulting in adsorption of the ligand onto the patterned relief of the PDMS stamp, which allows the selective binding of synNotch-expressing cells onto the stamped regions. In other implementations, alkanethiols are microprinted onto a gold surface, and the microprinted substrates are backfilled with PEG-thiol followed by immersion in a fusion protein solution, wherein the fusion protein comprises a ligand and fibronectin polypeptide, resulting in adsorption of the fusion protein onto alkanethiol-stamped regions, which allows the selective binding of synNotch-expressing cells onto the stamped regions.

In some embodiments, a predetermined pattern in one or more systems or combinations disclosed herein is produced by microfluidic patterning, wherein a feature characteristic of a microfluidic network is stamped to a substrate or to a culture surface. For example, by injecting a ligand solution through microchannels of a microfluidic device, a substrate in contact with the microfluidic device is exposed to the flow, resulting in patterning of the ligand onto the substrate. In another example, by injecting a ligand solution through microchannels of a microfluidic device, a culture surface with cells expressing synNotch receptor(s) is exposed to the flow, resulting in activation of synNotch-mediated transcription regulation in the cells in the pattern of the microchannels.

Further embodiments provide methods for differentiation or transdifferentiation of a quantity of the cells into two or more different types of cells, which include genetically co-expressing two or more different synthetic Notch receptors in the cells, wherein the two or more different synthetic Notch receptors differ at least by the extracellular domain in specifically binding a different ligand and by the transcriptional activator in driving expression of a different differentiation or transdifferentiation agent, which regulates differentiation or transdifferentiation of the cells into respective type of cells; and contacting the cell expressing the two or more different synthetic Notch receptors with a substrate having two or more different ligands, two or more different fusion proteins derived from respective ligand; thereby inducing cleavage of respective intracellular domain from each synthetic Notch receptor and causing respective transcriptional activator to induce expression of the differentiation or transdifferentiation agent in the cells, thereby co-differentiating or co-transdifferentiation the cells.

In some embodiments, the methods of co-differentiating or co-transdifferentiation result in a co-culture including the differentiated cell type(s) and optionally further including undifferentiated cells in a predetermined pattern. For example, for co-transdifferentiating fibroblast, a coculture system of fibroblasts, and myoblasts (or myoblast precursors) and/or endothelial cells (or endothelial precursors), is produced.

Additional embodiments provide a blood vessel-like structure based on myoblasts (or myoblast precursors) and/or endothelial cells (or endothelial precursors) in a predetermined pattern produced by one or more methods disclosed herein.

Some embodiments provide a blood vessel-like structure based on myoblasts (or myoblast precursors) and/or endothelial cells (or endothelial precursors), and fibroblasts, in a predetermined pattern produced by one or more methods disclosed herein.

Kits are also provided. In some embodiments, a kit includes a substrate and a ligand, a fusion protein comprising the ligand and an extracellular matrix polypeptide, or a first cell secreting or expressing on cell surface the ligand or the fusion protein, or a combination thereof, positioned in a predetermined pattern on the surface of and/or inside the substrate.

In some embodiments, a kit includes a substrate; a ligand, a fusion protein comprising the ligand and an extracellular matrix polypeptide, or a first cell secreting or expressing on cell surface the ligand or the fusion protein, or a combination thereof, positioned in a predetermined pattern on the surface of and/or inside the substrate; and a polynucleotide encoding a synNotch receptor.

In some embodiments, a kit includes a substrate; a ligand, a fusion protein comprising the ligand and an extracellular matrix polypeptide, or a first cell secreting or expressing on cell surface the ligand or the fusion protein, or a combination thereof, positioned in a predetermined pattern on the surface of and/or inside the substrate; and a cell engineered to express a synNotch receptor.

In some embodiments, a kit includes a substrate; a ligand, a fusion protein comprising the ligand and an extracellular matrix polypeptide, or a first cell secreting or expressing on cell surface the ligand or the fusion protein, or a combination thereof, positioned in a predetermined pattern on the surface of and/or inside the substrate; a polynucleotide encoding a synNotch receptor; and one or more reagents for transfecting a cell.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Activation of synNotch from Particles and Cell-Generated ECM

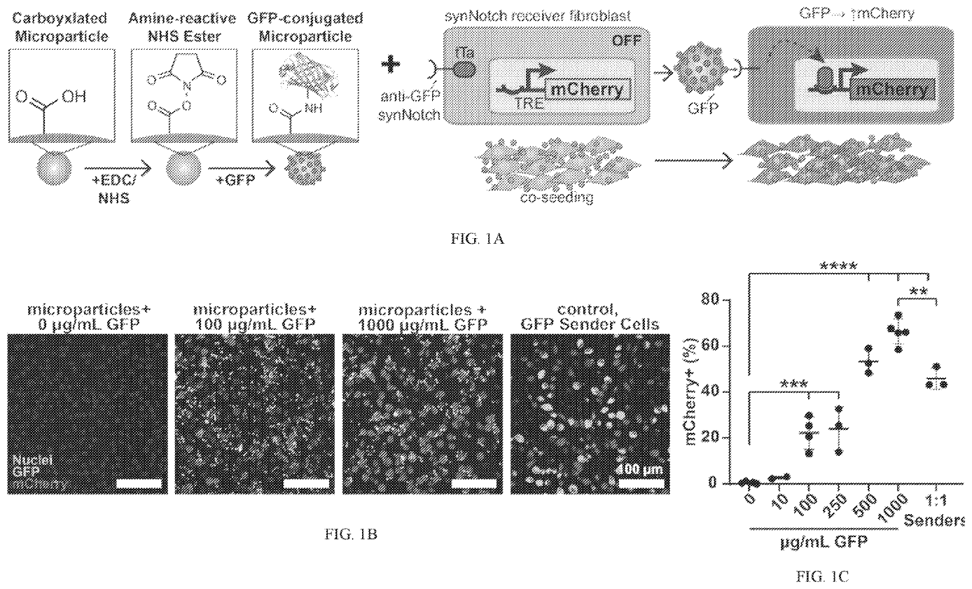

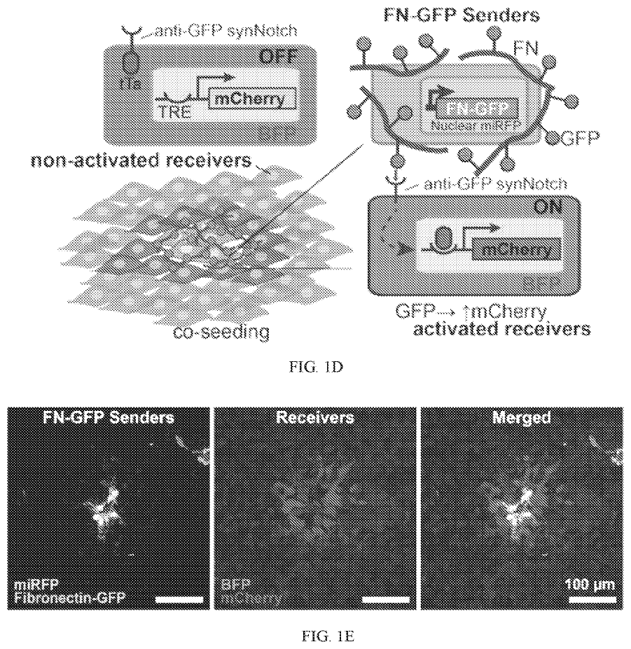

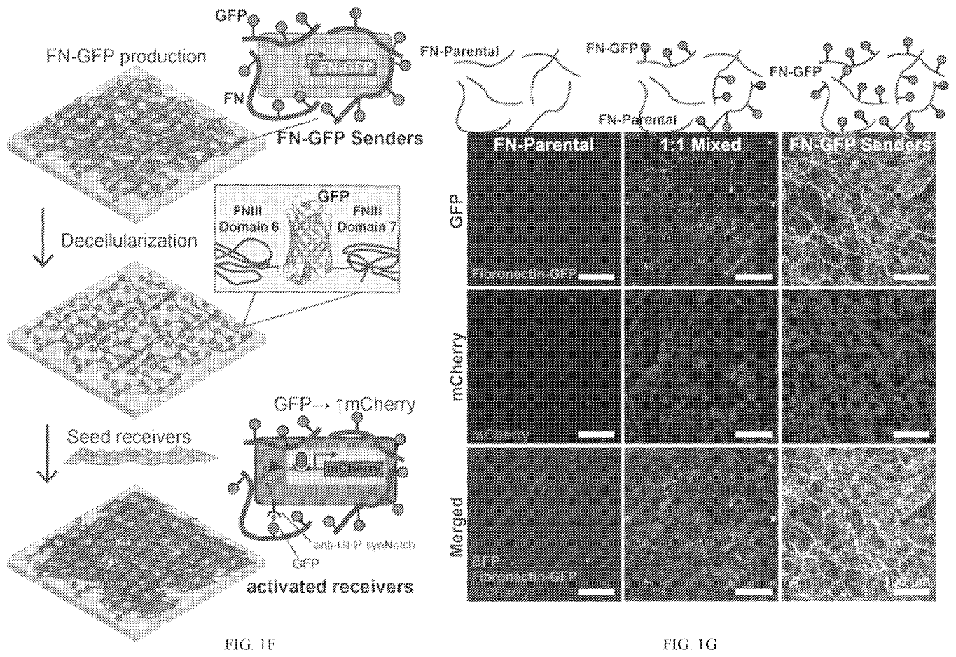

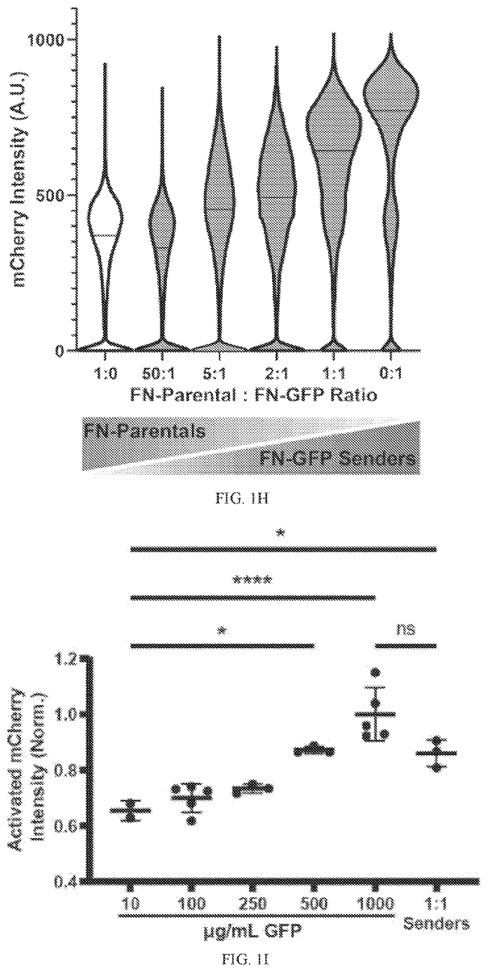

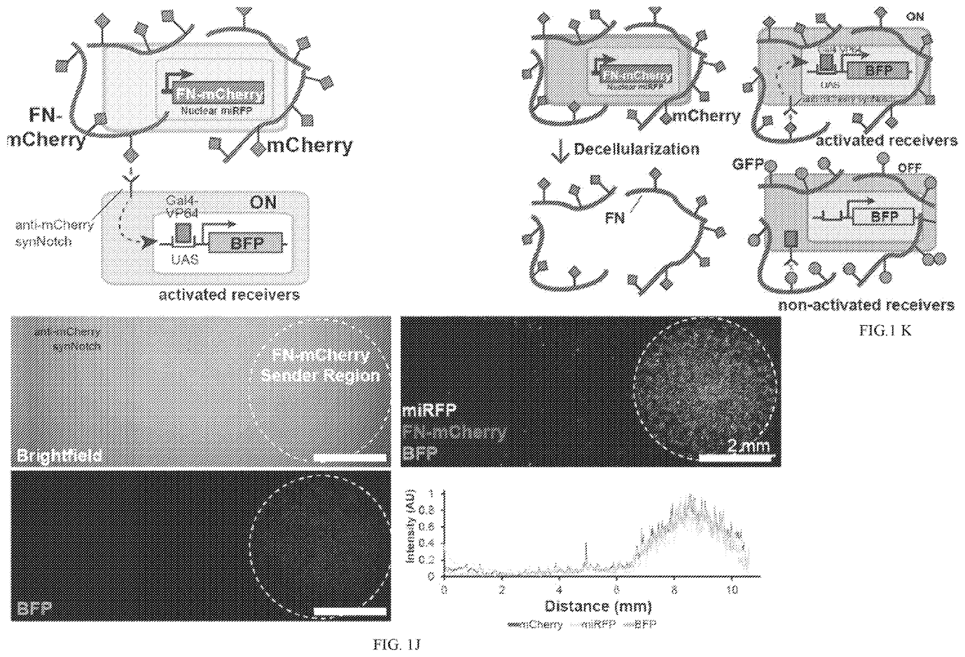

To evaluate synNotch activation by ligands presented on materials, we first used microparticles in suspension to present ligands semi-analogously to the presentation of ligands on the membranes of sender cells. We tethered GFP to carboxyl-modified microparticles of different diameters (2 μm-10 μm) using an EDC/NHS reaction. This approach allows for different amounts of GFP to be loaded by simply adjusting the concentration of GFP in the conjugation reaction. Then we added these microparticles to a monolayer of receiver fibroblasts (L929 cells) that were engineered to express an anti-GFP/tTA synNotch receptor and its mCherry reporter gene (FIG. 1A). mCherry fluorescence at 24-hour post-seeding increased with increasing concentration of GFP loaded onto the microparticles for all particle diameters, and was absent when cells were presented with unmodified particles (FIGS. 1B, 1C, 1I). Importantly, 5 μm microparticles loaded with 500 or 1000 μg/mL GFP induced mCherry in the receiver fibroblasts at a level similar to GFP-presenting sender cells co-cultured with receiver cells at a 1:1 ratio, indicating that synthetic ligands conjugated to microparticles can activate synNotch receptors to a similar extent as synthetic ligands presented by sender cells.

We next asked if synNotch receptors could be activated by synthetic ligands presented on ECM fibers produced by cells. To test this, we genetically engineered mouse embryonic fibroblasts (3T3 cells) to produce a fusion protein of fibronectin with GFP (FN-GFP, preparation described by Ohashi et al. in Journal of Biological Chemistry 286, 39188-39199 (2011)). These cells were also engineered to express a far-red fluorescent nuclear reporter protein. We hypothesized that these FN-GFP sender cells would deposit an ECM containing synthetic ligands that would signal to receiver cells. To test this, we co-cultured a low amount of FN-GFP sender cells alongside receiver fibroblasts expressing anti-GFP/tTA synNotch receptors that activate mCherry (FIG. 1D). At 72-hour post-seeding, we observed mCherry expression only in receiver cells that are near to FN-GFP sender cells (FIG. 1E), indicating that the anti-GFP synNotch receptor is locally activated in response to FN-GFP embedded in the ECM. We then tested if cell-deposited FN-GFP matrices can activate receiver cells after the sender cells are removed. To do so, we cultured FN-GFP sender cells as a monolayer for 8 days and subsequently performed decellularization to remove all cellular components while preserving the extracellular matrix (FIG. 1F). Receiver cells cultured on the decellularized matrices for 48 hours expressed mCherry, indicating that synthetic ligands embedded in the ECM remained functional through the decellularization process. To tune the level of synNotch receptor activation by decellularized matrices, we co-cultured FN-GFP sender cells with the unmodified parental 3T3 cells at various ratios. We similarly decellularized the co-cultured tissues and then seeded the decellularized matrices with receiver cells. mCherry intensity scaled with the ratio of parental cells to GFP-FN sender cells in the original tissue (FIG. 1G, 1H), demonstrating tunability of activation of synNotch via cell-produced ECM fibers.

One advantage of synthetic receptors is that they can be engineered to both recognize distinct input ligands and drive distinct cellular responses. This feature has been used to generate a library of orthogonal synNotch receptors and pathways that function independently from each other and from endogenous receptors and pathways. To test if activation of synNotch receptors by matrix-presented synthetic ligands is generalizable to other ligand-receptor pairs, we generated FN-mCherry sender cells as well as corresponding receiver cells with anti-mCherry synNotch/Gal4 receptors that induce a BFP reporter gene upon activation. Similar to FN-GFP sender cells, FN-mCherry sender cells activate receiver cells in co-culture and upon decellularization (FIG. 1J). We also observed that anti-mCherry receiver cells were not activated by FN-GFP decellularized matrices, illustrating the orthogonality of receptor activation by matrix-presented synthetic ligands (FIG. 1K). Overall, these data demonstrate that synNotch receptors can be robustly, tunably, and modularly activated by ligands presented on cell-produced ECM fibers.

Activation of synNotch from Hydrogels in 2D and 3D

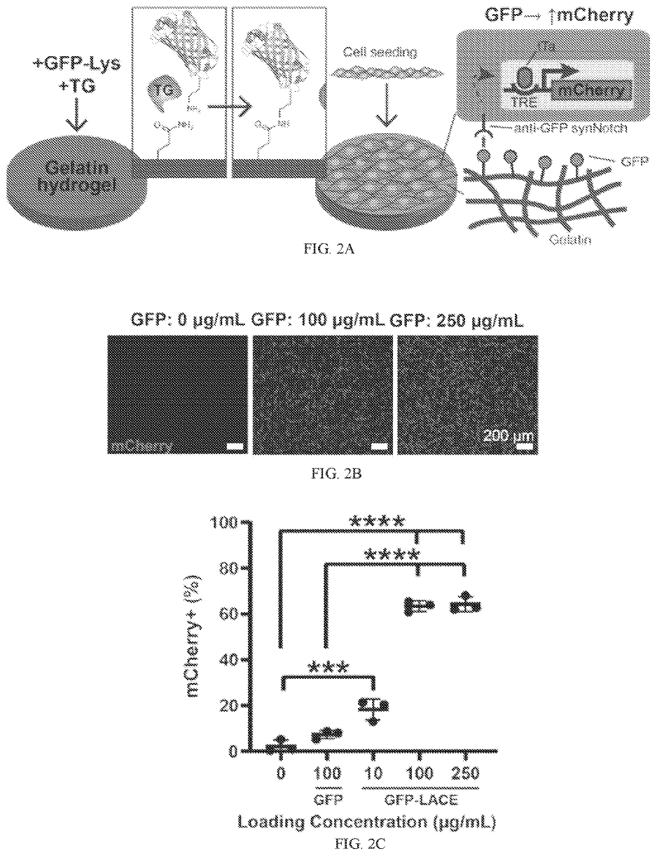

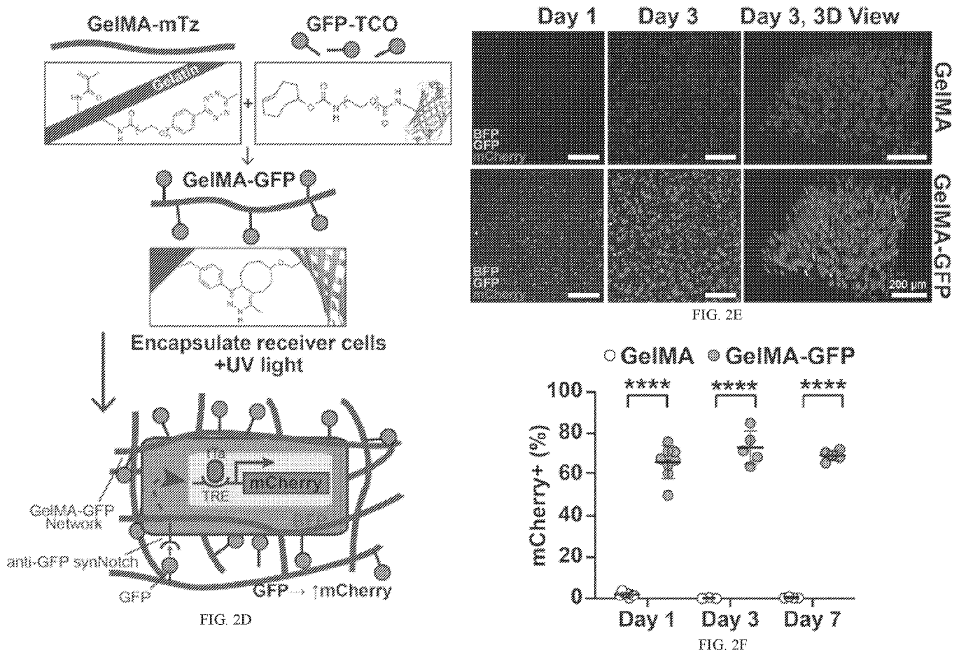

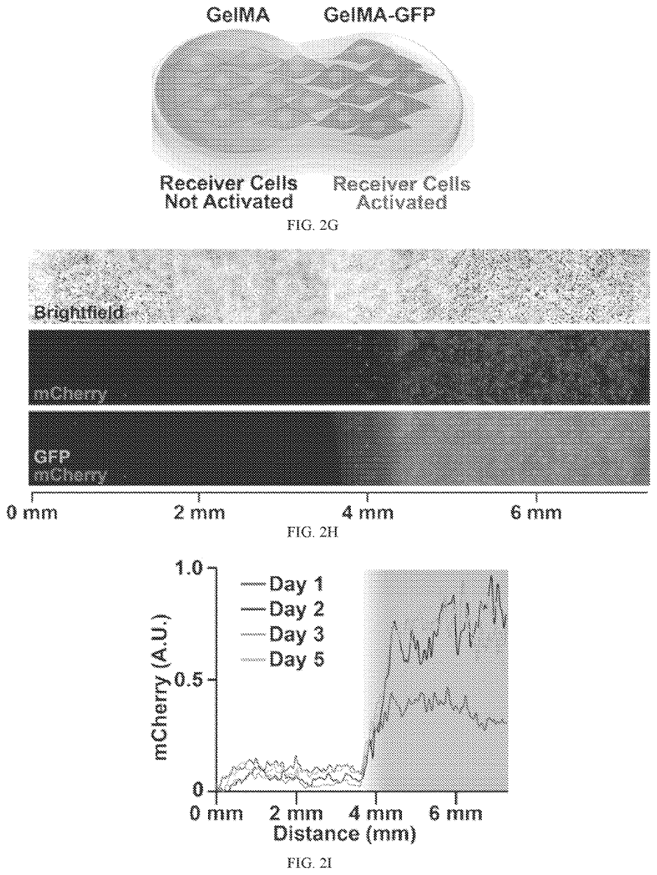

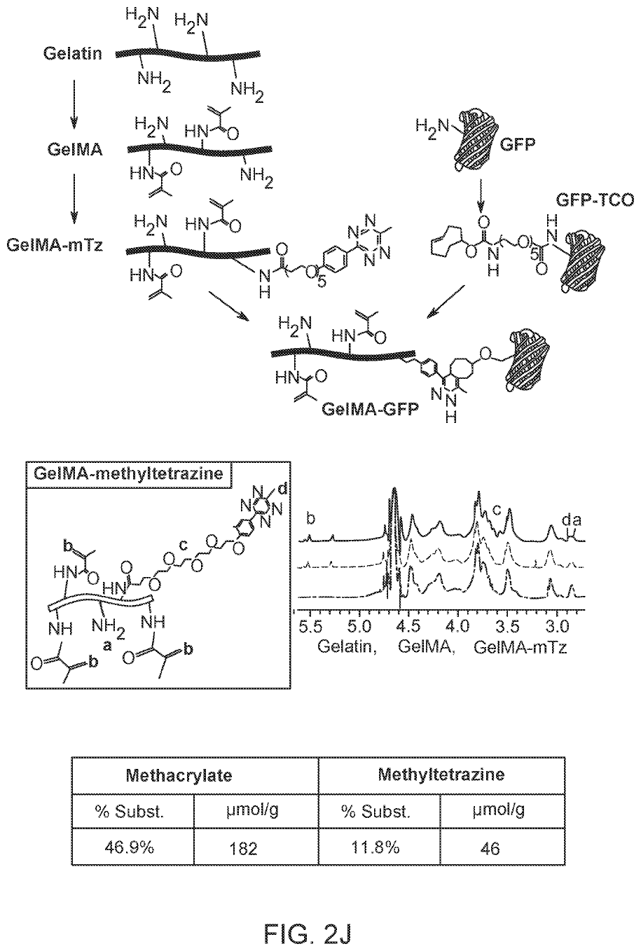

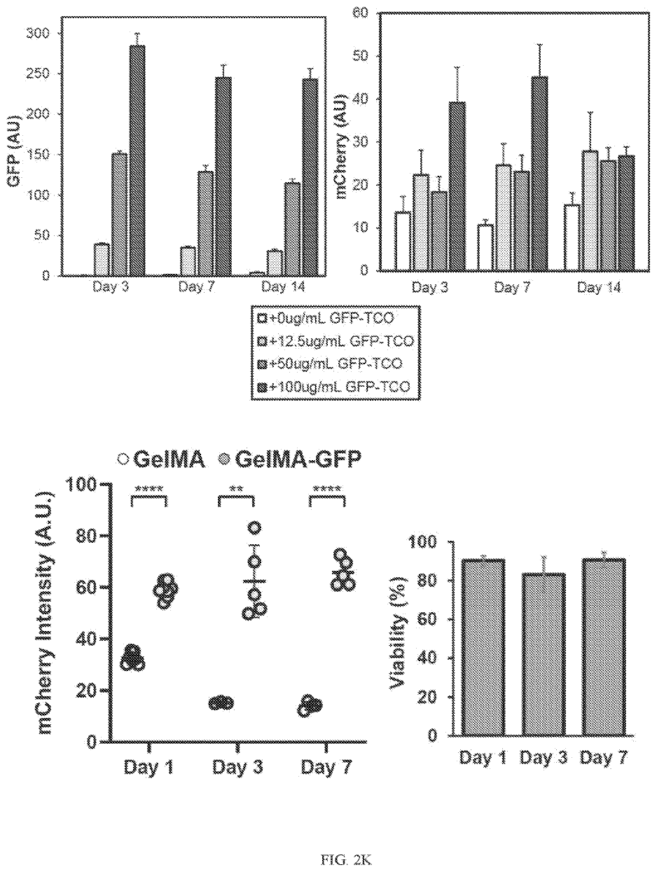

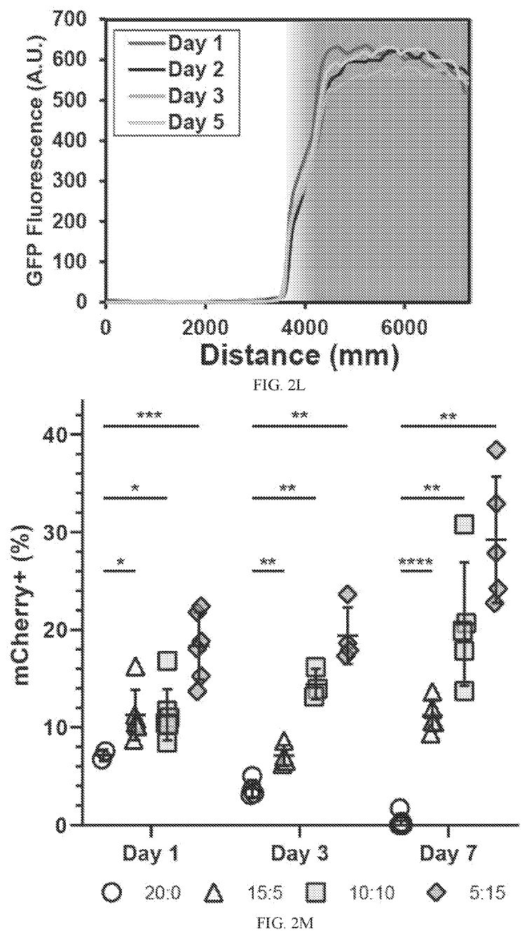

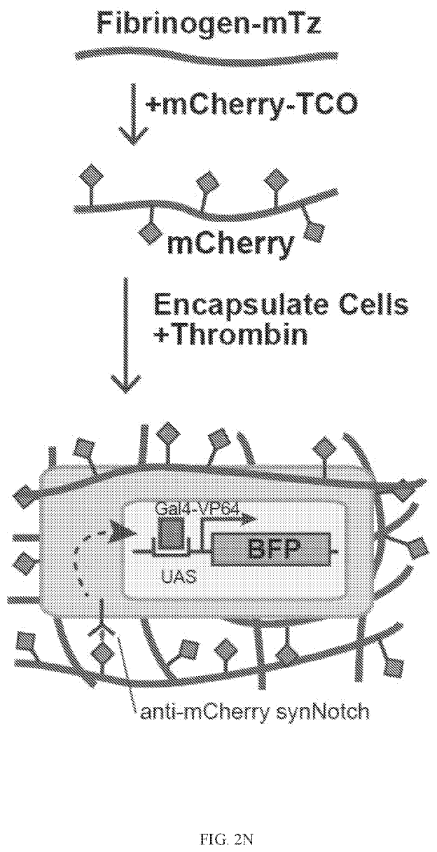

To improve user control and tunability, we next tested if synNotch could be activated by ligands presented on purified ECM fibers processed into hydrogel biomaterials. As a first step, we attempted to activate synNotch receptors in cells cultured on the surface of matrix-derived hydrogels. We fabricated slabs of gelatin hydrogels enzymatically cross-linked with transglutaminase, an enzyme that cross-links glutamine and lysine residues. We next sought to conjugate GFP onto the hydrogel surface with transglutaminase. However, GFP is weakly susceptible to transglutaminase because the glutamine and lysine residues of globular proteins are relatively inaccessible. Thus, we synthesized GFP with a short C terminus LACE peptide tag (GFP-LACE) to provide accessible lysine residues. (Lysine acylation using conjugating enzymes (LACE) is described by Hofmann et al. in Nature Chemistry 12, 1008-1015 (2020).) We then treated gelatin hydrogels with a solution of GFP-LACE and transglutaminase to conjugate GFP onto the surface (FIG. 2A). When receiver cells with anti-GFP synNotch/tTA receptors that activate mCherry were cultured on the GFP-gelatin hydrogels, mCherry intensity increased in a GFP dose-dependent manner (FIG. 2B, 2C). Thus, synNotch receptors can be activated by synthetic ligands presented on the surface of matrix-derived hydrogels.

Next, we attempted to activate synNotch receptors in cells embedded in 3-D matrix-derived hydrogels that present synthetic ligands. To do so, we developed a click chemistry method to conjugate synthetic ligands to hydrogels (FIG. 2D). Briefly, GFP was modified with trans-Cyclooctene (TCO) NHS ester to generate GFP-TCO moieties. In parallel, gelatin polymer was modified with methacrylate (MA) groups for photo-cross-linking and methyltetrazine (mTz) to generate GelMA-mTz (FIG. 2J). These coordinated substitutions allow for facile conjugation of TCO-modified protein ligands to the mTz-modified hydrogel polymer backbone via rapid click reaction after mixing. Combining GelMA-mTz with GFP-TCO generated GelMA-GFP, which could then be photocrosslinked into a hydrogel that demonstrated retention of the GFP ligand for over seven days. GelMA hydrogels maintain encapsulated cell viability for at least seven days maintaining ~90% viability when quantified via Live/Dead staining (FIG. 2K, bottom right).

We then embedded anti-GFP receiver fibroblasts in GelMA-GFP hydrogels via photocrosslinking. As shown in FIGS. 2E and 2F, mCherry expression in receiver cells significantly increased in GelMA-GFP hydrogels but not in unmodified GelMA hydrogels (FIG. 2K). Up to 70% of the receiver cell population within GelMA-GFP had sustained activation for up to 7 days. In contrast, when we attempted to activate synNotch receiver cells via sender cells co-embedded in a GelMA hydrogel, only 30% activation of the receiver cell population was observed (FIG. 2M). To demonstrate spatial confinement of activation, we next encapsulated receiver fibroblasts via manual pipetting in a biphasic GelMA hydrogel, where only half of the hydrogel contained GFP. Due to the covalent linkage between GFP and GelMA, the spatial position of GFP was maintained over time and the GFP did not diffuse through the hydrogel (FIG. 2L). As shown in FIGS. 2G-2I, mCherry activation was similarly spatially restricted to the GelMA-GFP region over time, demonstrating that the GFP ligand conjugated to the hydrogel activated synNotch only in the regions where it was originally positioned. To validate the modularity of this method, we also engineered fibrinogen-mCherry constructs via a similar click chemistry approach (FIG. 2N). We then embedded anti-mCherry/Gal4 synNotch receiver cells that activate BFP in these hydrogels. Receiver cells were activated only in fibrinogen-mCherry hydrogels but not unmodified fibrinogen hydrogels. Collectively, these results demonstrate that matrix-derived hydrogels can be covalently conjugated with synthetic ligands to generate 2-D or 3-D materials capable of locally activating receiver cells.

Spatial Activation of synNotch Via Microcontact Printing

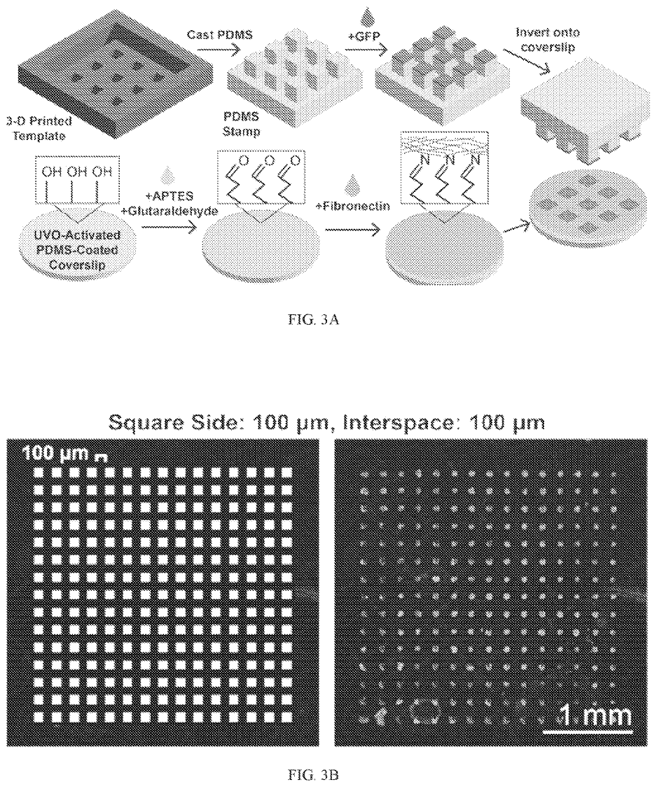

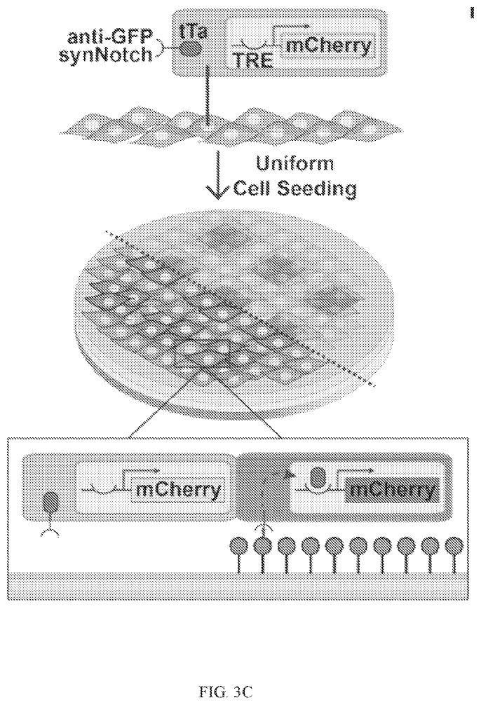

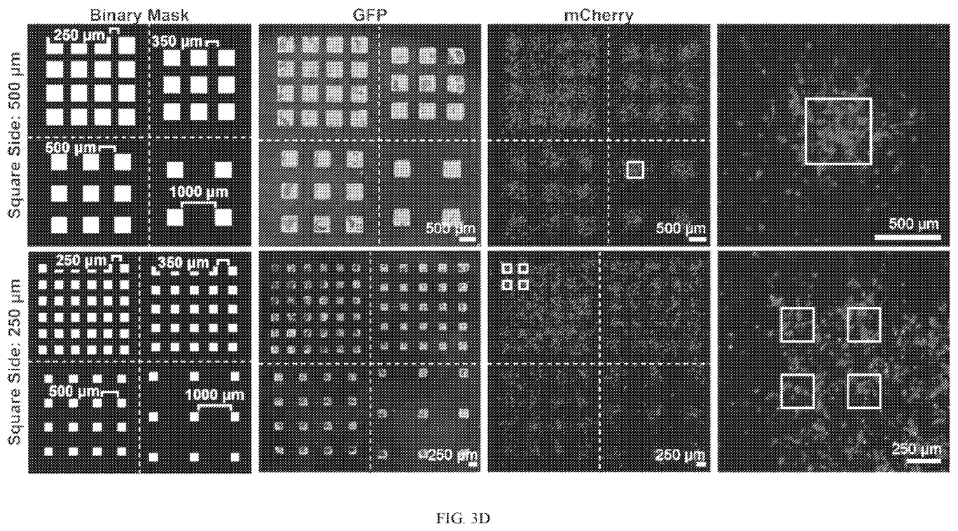

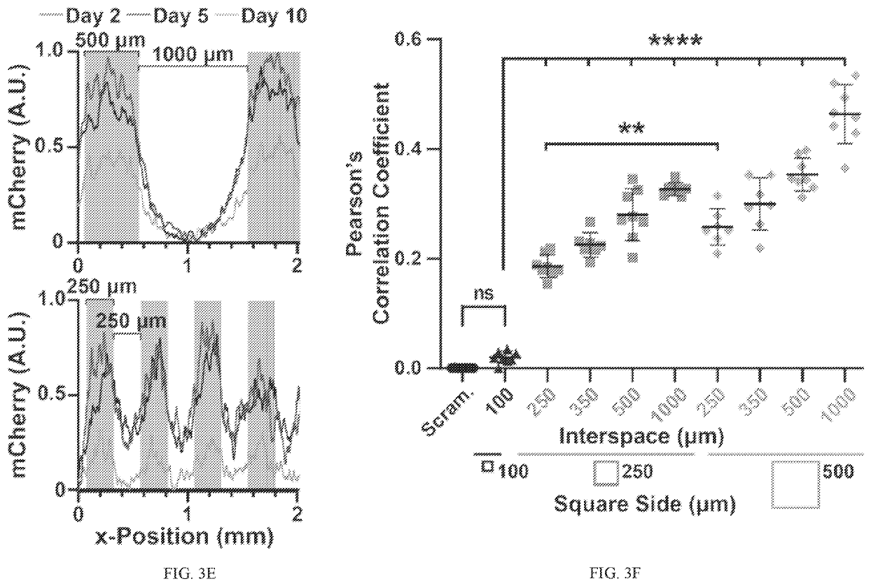

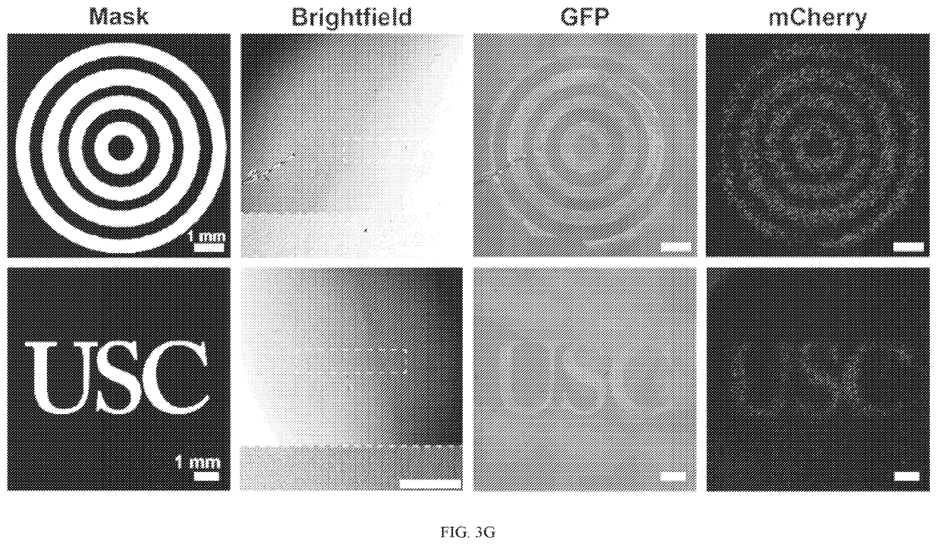

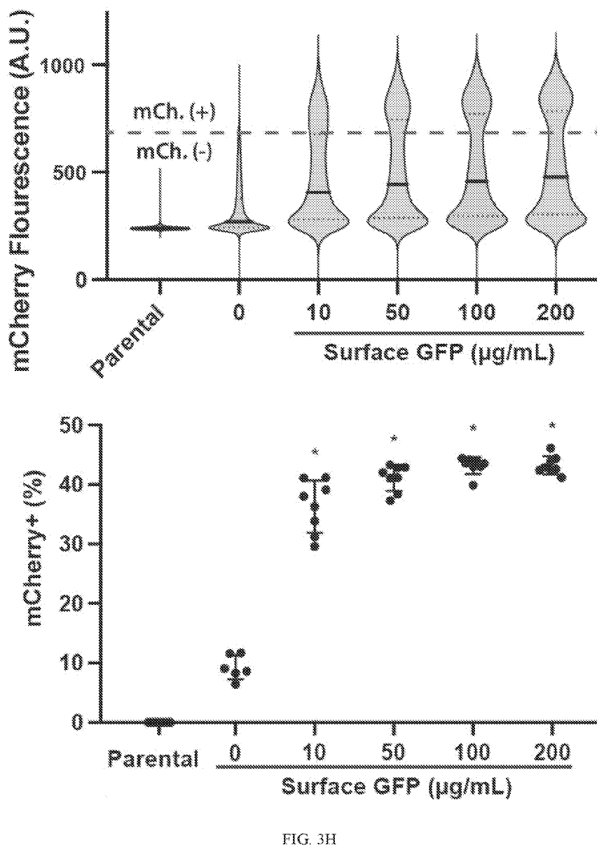

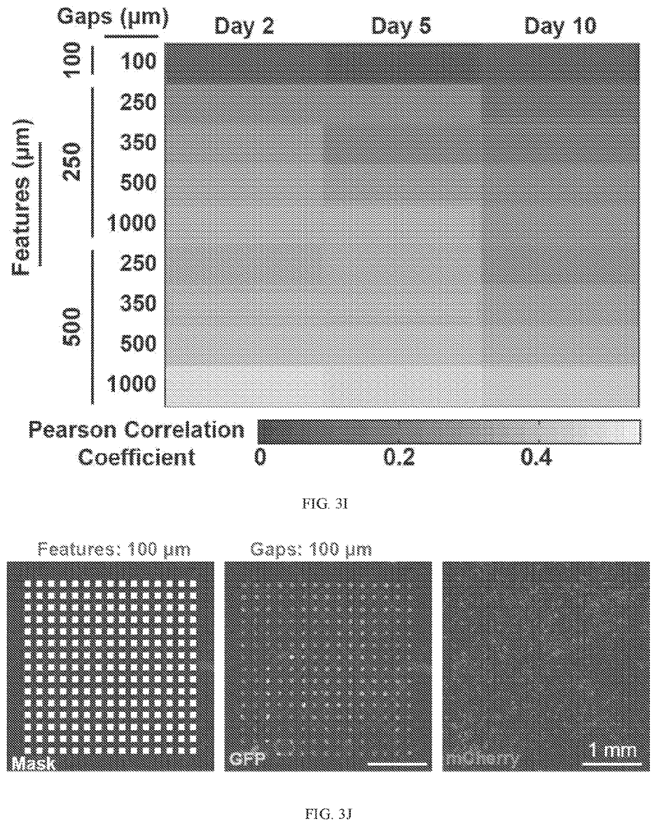

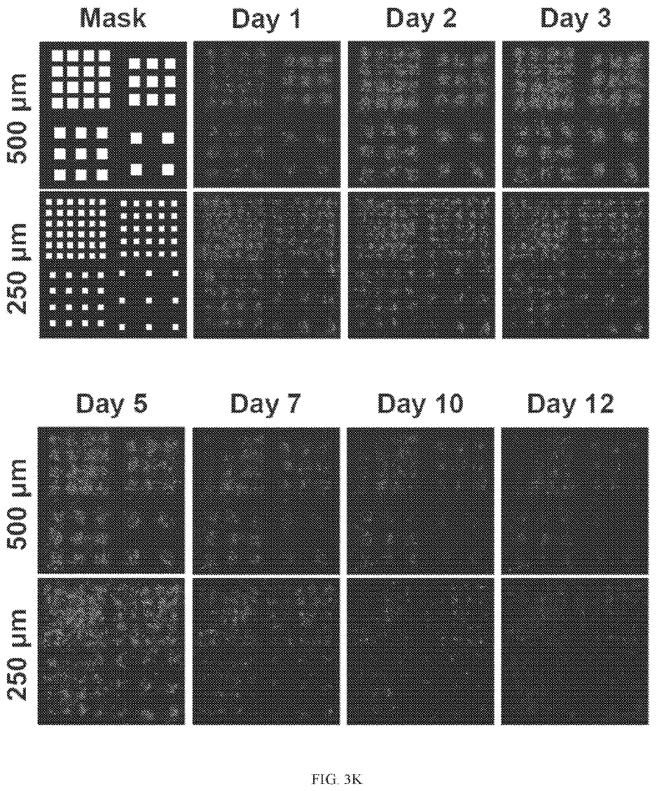

Our next goal was to dictate synNotch activation patterns within multicellular tissue constructs at a spatial resolution similar to the cellular length scale. To achieve this, we adapted microcontact printing techniques designed to transfer microscale patterns of proteins (classically ECM proteins) onto culture surfaces. Our goal was to microcontact print GFP onto uniformly cell-adhesive surfaces (FIG. 3A). To achieve this, we treated PDMS-coated coverslips with (3-aminopropyl)triethoxy silane (APTES) and glutaraldehyde to induce covalent bonding of proteins and then coated the surface with fibronectin for uniform cell adhesion. To optimize the transfer of GFP onto the fibronectin layer, we created simple, featureless PDMS stamps by cutting cylinders from PDMS using a biopsy punch. We coated and incubated these stamps with 0-200 μg/mL GFP solutions and then inverted them onto fibronectin-coated coverslips. Finally, we seeded coverslips with receiver cells expressing anti-GFP/tTA synNotch receptors that activate an mCherry reporter. These cells formed a confluent monolayer and demonstrated a GFP dose-dependent increase in mCherry fluorescence that saturated at roughly 100 μg/mL GFP (FIG. 3H), indicating that surfaces dual-functionalized with fibronectin and GFP maintained cell adhesion and activated synNotch.

To induce activation of synNotch in small groups of cells within a multicellular tissue, we next developed an approach to microcontact print arrays of GFP squares with features ranging from 100 μm to 1 mm. PDMS stamps for microcontact printing are classically cast on silicon wafer templates fabricated by cleanroom-based photolithography. However, this approach is not suitable for our feature sizes because they are large (100 μm to 1 mm) relative to the height of photoresist conventionally used for photolithography (1-10 μm). PDMS stamps with high feature to height ratios are susceptible to buckling and transfer of GFP outside the intended regions. To overcome this, we used a digital light processing (DLP) 3-D printer to rapidly print templates with taller features in a photocrosslinkable resin. We first 3-D printed a template comprising an array of 100 μm sided-squares with 100 μm interspaces, which is roughly the resolution limit of the 3-D printer. The height of the features was set as 100 μm to minimize buckling. As shown in FIG. 3B, PDMS stamps fabricated in this way could successfully transfer GFP onto covalently coated FN coverslips in the intended 100 μm×100 μm pattern, demonstrating successful microcontact printing using PDMS stamps cast on 3D printed templates. Successful transfer of GFP may refer to zero or undetectable amount of GFP outside intended region.

We next used these techniques to fabricate stamps and microcontact print arrays of GFP squares with sides ranging from 250 μm to 1000 μm and interspaces of 250 μm or 500 μm onto PDMS-coated coverslips pre-coated with fibronectin. The feature height for these stamps ranged from 100 μm to 500 μm, depending on square sizes and interspaces. Microcontact printed surfaces were then seeded with receiver cells with anti-GFP/tTA synNotch receptors that activate an mCherry reporter (FIG. 3C). After two days, mCherry expression was detected within the multicellular tissue in patterns that overlapped with the original design to different extents, depending on the pattern (FIG. 3D, 3E). To quantify the spatial fidelity of synNotch activation, we calculated the Pearson's correlation coefficient between the binary pattern design and the mCherry images (FIGS. 3F and 3I). The correlation coefficient was highest for tissues with the largest squares (500 μm sides) and largest interspaces (1000 μm). The correlation coefficient decreased as features and/or gaps decreased. However, for all tissues with square sizes and interspaces greater than 100 μm (FIG. 3J), the correlation coefficient between the mCherry image and the binary pattern was significantly higher compared to the correlation coefficient between the mCherry image and a scrambled binary pattern with the same number of white pixels. The correlation also decreased with time due to weakening of reporter activation (FIG. 3E, 3K). Together, these data indicate that a preferable feature size for this approach is approximately 250 μm or greater. Based on this observation, we designed other arbitrary patterns with minimal feature sizes of 250 μm, including concentric circles and letters. Qualitatively, we observed similar agreement between the binary pattern, GFP fluorescence, and mCherry fluorescence (FIG. 3G), demonstrating versatility of pattern designs.

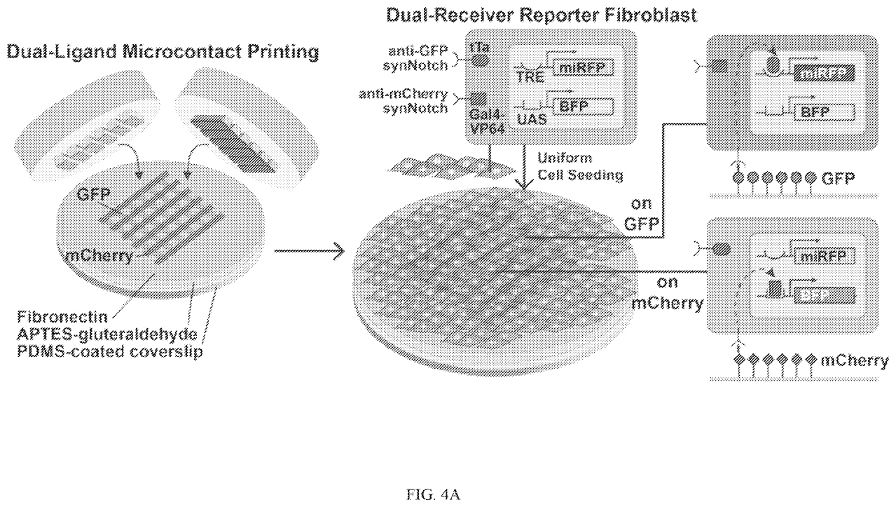

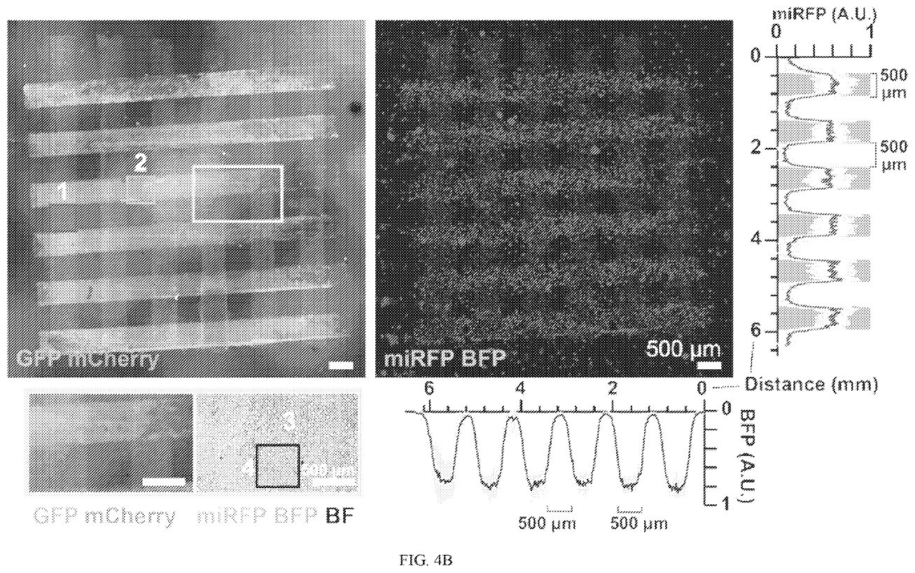

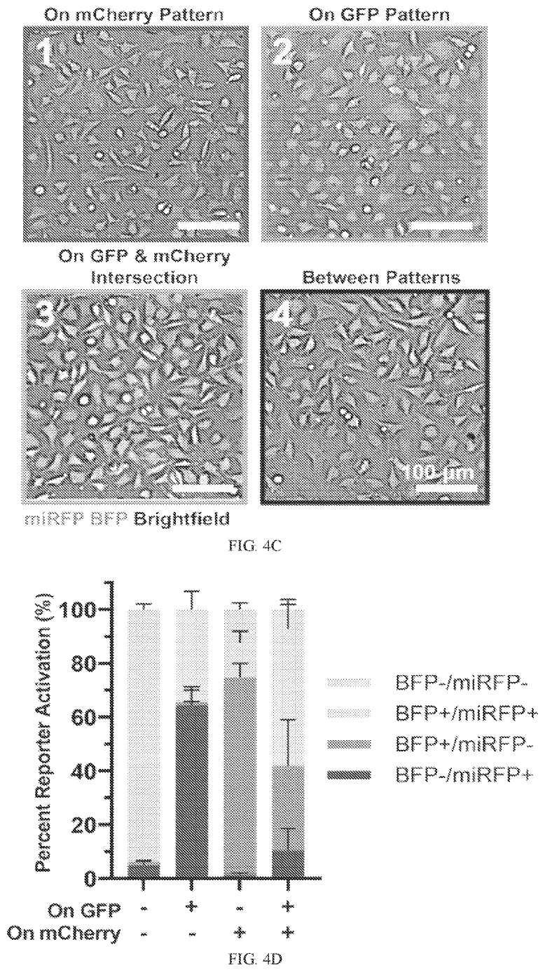

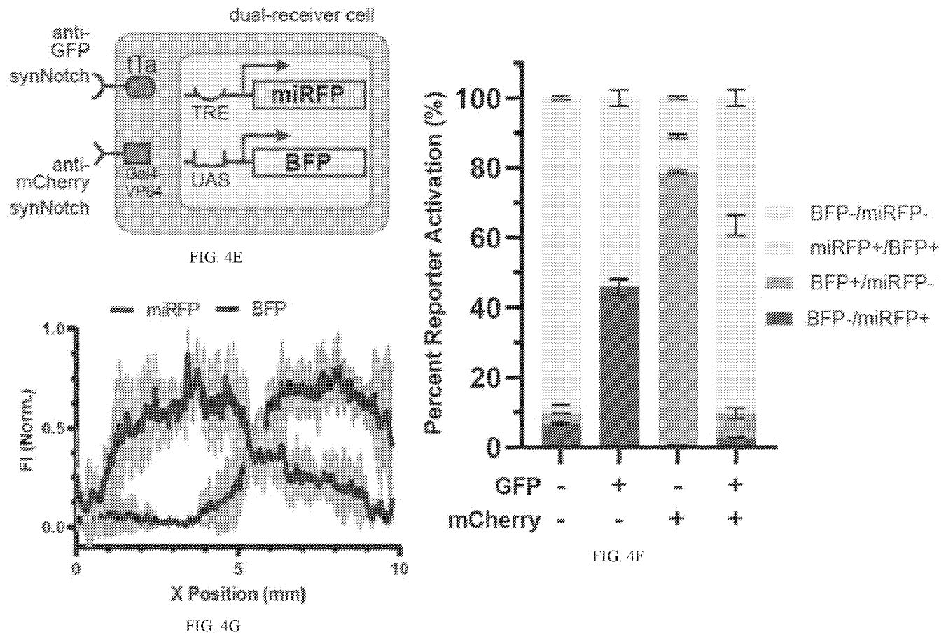

Our next goal was to scale-up this approach to spatially activate multiple distinct genetic programs in the same multicellular tissue. We asked if culturing dual-receiver cells on a surface patterned with two synthetic ligands in distinct arrangements would generate a tissue with corresponding patterns of distinct genetic programs (FIG. 4A). We first generated a dual-receiver fibroblast cell line (L929) that harbors an anti-GFP/tTA synNotch receptor that activates an miRFP reporter and an anti-mCherry/Gal4 synNotch that activates a BFP reporter (FIG. 4E). To validate the responses to synthetic ligands of these cells, we seeded them on a culture surface microcontact printed with a uniform layer of GFP, mCherry, or both. As shown in FIG. 4F, miRFP was expressed only on GFP surfaces and BFP was expressed only on mCherry surfaces, demonstrating orthogonal activation of the two pathways. On surfaces with both GFP and mCherry, both miRFP and BFP were expressed, indicating activation of both pathways. Next, to prototype the generation of spatial patterns of gene expression starting from a uniform population of dual-receiver cells, we adsorbed GFP from a droplet in one corner of a culture surface and a droplet of mCherry in the opposing corner. Dual-receiver cells cultured uniformly on the surface activated miRFP and BFP in a spatial pattern corresponding to the GFP and mCherry droplets, respectively, demonstrating macroscale spatial control over the activation of two synNotch pathways in one cell population (FIG. 4G). Finally, to provide more precise spatial control over the patterns, we microcontact printed an array of 500 μm-wide rows of GFP with 500 μm interspacing. We then stamped perpendicular mCherry rows by manually positioning the orientation of the stamp (FIG. 4B). When seeded with dual-receiver cells, we observed rows of miRFP-expressing cells perpendicular to rows of BFP-expressing cells (FIG. 4B). At the GFP and mCherry intersections, cells expressed both miRFP and BFP (FIG. 4C), indicating activation of both synNotch pathways, generating 4 reporter “states” for the initially uniform population of engineered cells (BFP−/miRFP−, BFP−/miRFP+, BFP+/miRFP−, BFP+/miRFP+) within the 1.5 mm² tissue. Additionally, we quantified the percent of BFP and miRFP expression in cells on different regions of the pattern with image analysis (FIG. 4D). Roughly 60-70% of dual-receiver cells on a region with a single ligand (GFP or mCherry) expressed the matching reporter (miRFP or BFP, respectively). On the GFP and mCherry intersections, roughly 50% of dual-receiver cells expressed both BFP and miRFP. These values were similar to the percent reporter activation measured by flow cytometry in dual-receiver fibroblasts cultured on surfaces uniformly adsorbed with one or both ligands (FIG. 4F). Thus, two independent synNotch genetic programs can be spatially controlled by culturing dual-receiver cells on user-defined patterns of the two synthetic ligands, to generate a multicellular tissue with up to four spatially controlled reporter gene expression states.

### Spatial Control of Concurrent Differentiation to Skeletal Muscle and Endothelial Cell Precursors

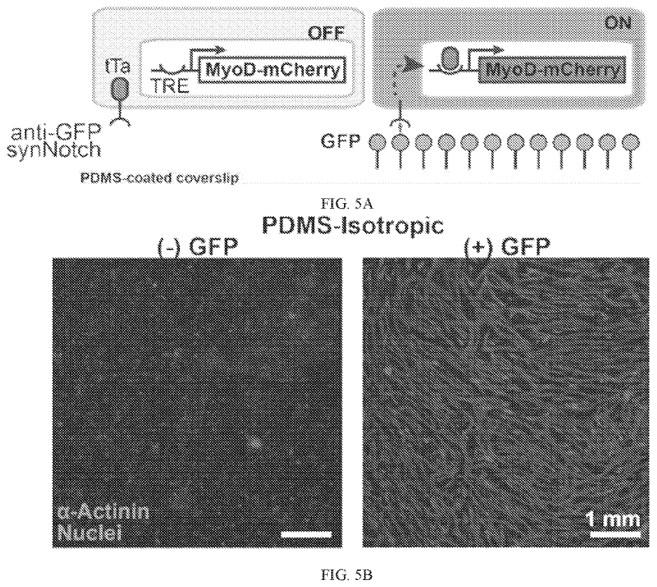

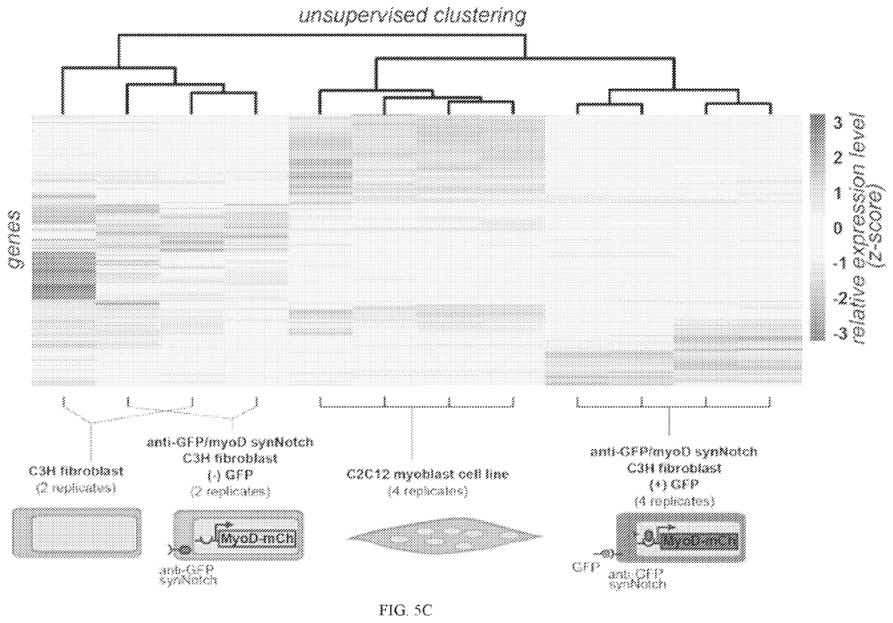

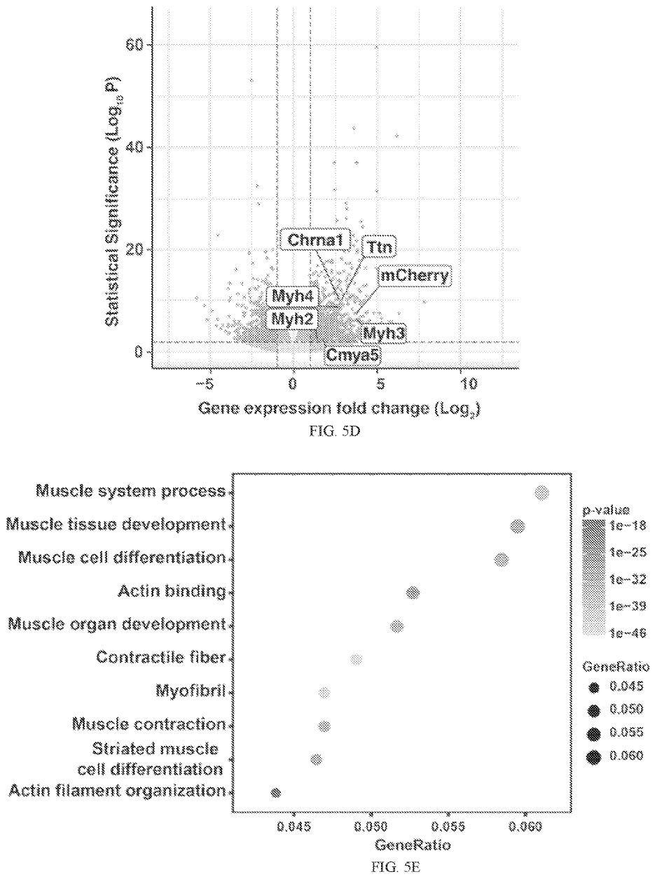

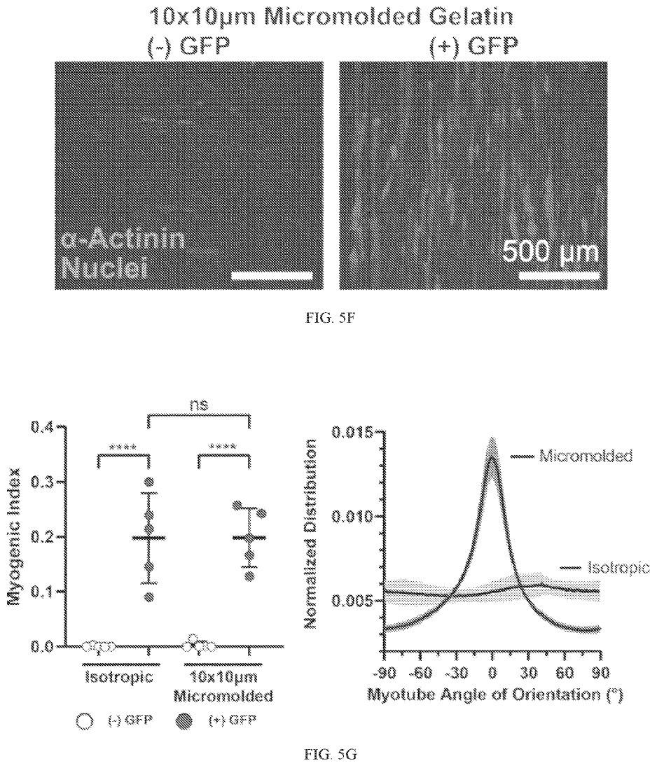

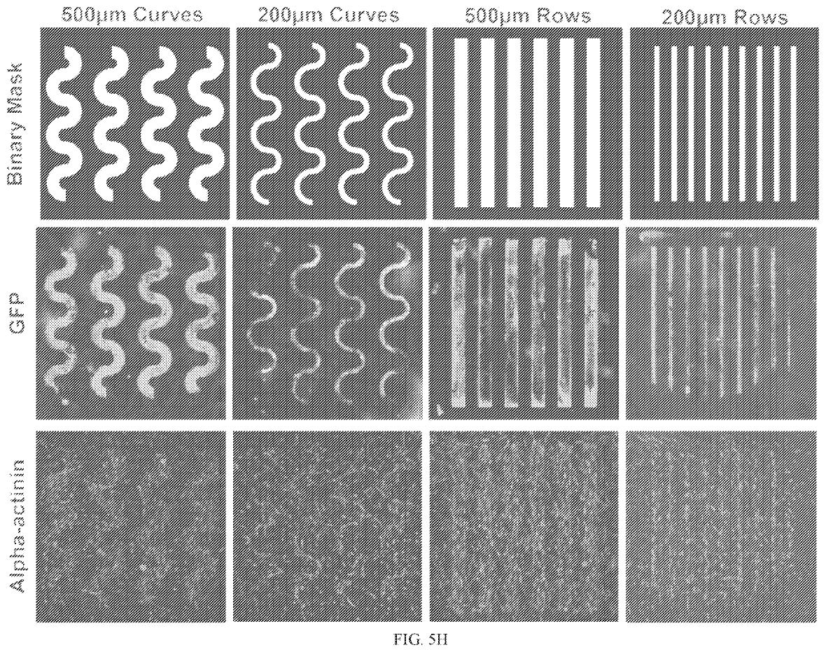

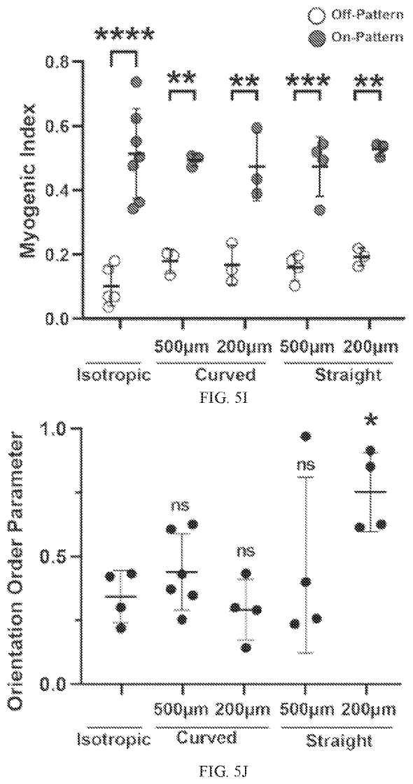

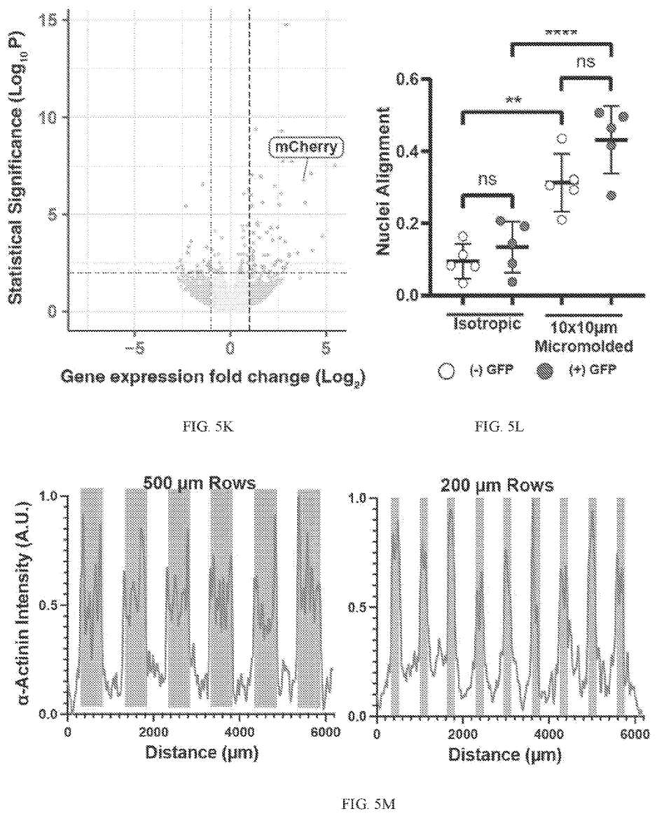

We next tested if synthetic ligands presented by materials could drive overexpression of functional transcription factors that induce transdifferentiation. We first generated a receiver fibroblast cell line (C3H) expressing an anti-GFP/tTA synNotch receptor that activates myoD (FIG. 5A). When these receiver cells were cultured on surfaces uniformly printed with GFP, they transdifferentiated to multinucleated, α-actinin positive myotubes (FIG. 5B). To further characterize changes in phenotype, we performed bulk RNA sequencing on unmodified C3H fibroblasts, receiver cells cultured on surfaces with or without GFP, and C2C12 myotubes. We observed that culturing receiver cells on GFP surfaces led to 3,064 differentially expressed genes. According to hierarchical clustering, receiver cells on GFP were most similar to C2C12 myotubes (FIG. 5C). Receiver cells on GFP also over-expressed several muscle-specific genes, such as Myh2, Myh4, and Ttn, and down-regulated expression of fibroblast genes, such as Col1a1 and Pdgfrb (FIG. 5D). GO-term analysis indicated that several pathways related to muscle development and differentiation were enriched in receiver cells on surfaces with GFP compared to without GFP (FIG. 5E). In contrast, receiver cells expressing an anti-GFP/tTA synNotch receptor that activates mCherry did not over-express muscle-specific genes or pathways, and only led to 33 differentially expressed genes, when cultured on surfaces with or without GFP (FIG. 5K). Together, these data indicate that surfaces with GFP specifically induced the transdifferentiation of receiver cells expressing an anti-GFP synNotch receptor that activates MyoD to myogenic precursors.

Our next goal was to combine the synNotch receptor technology with surface micropatterning to engineer aligned muscle tissue. We asked if gelatin hydrogel surface could be used to both transdifferentiate and align synNotch-induced myotubes. We constructed gelatin hydrogels that are either isotropic or micromolded with 10 μm ridges separated by 10 μm spacing and then enzymatically conjugated GFP to the surface using the procedure described in FIG. 2A. Receiver cells cultured on GFP transdifferentiated to α-actinin positive myotubes, independent of surface topography, and receiver cells consistently aligned to micromolded ridges (FIG. 5A, 5B, 5L), independent of activation state. However, only receiver cells cultured on micromolded GFP hydrogels fused into aligned myotubes (FIG. 5A, 5B, 5L), demonstrating that transdifferentiation and cell alignment were controlled independently. We did observe a slight but non-significant increase in nuclei alignment for cells cultured on micromolded gelatin hydrogels with GFP compared to without GFP (FIG. 5L), possibly because cell fusion induced by MyoD caused a modest improvement in cell alignment.

Another approach for engineering aligned muscle tissues is to culture muscle cells on microcontact printed lines of matrix proteins. We tested if this approach was compatible with synNotch by microcontact printing lines of a mixture of fibronectin and GFP. When the same receiver cells were cultured on these surfaces, they transdifferentiated into aligned myotubes (FIG. 5L), indicating that microcontact printing matrix proteins and synthetic ligands can also be used to both control tissue architecture and transdifferentiation.

In the approaches described above, a population of fibroblasts was uniformly transdifferentiated to myoblasts. Our next goal was to selectively transdifferentiate fibroblasts to myoblasts in a spatially controlled manner as a first step towards generating tissues with multiple distinct cell types arranged in prescribed patterns. To achieve this, we used the approach described in FIG. 3A to microcontact print rows of GFP on fibronectin-coated surfaces. To test if we could achieve spatially controlled differentiation, we printed thin or thick, curved or straight, rows and then seeded the printed surfaces with fibroblasts harboring an anti-GFP synNotch receptor that activates myoD (FIG. 5E). After three days, we fixed and stained tissues for α-actinin and quantified the myogenic index on and off the pattern by using the binary pattern as a mask (FIG. 5F). Myogenic index was significantly higher on-pattern compared to off-pattern for all geometries, demonstrating local geometric control of transdifferentiation. We also quantified the coherency of the tissues as a proxy for alignment and observed higher coherency for tissues on the straight rows compared to the curved rows, where 200 μm rows improved myotube orientation (FIG. 5G; 5M). Thus, we can selectively transdifferentiate fibroblasts to myoblasts in a geometrically prescribed way while also controlling the global alignment of the tissue, demonstrating that we can separately and concurrently control local differentiation and tissue architecture.

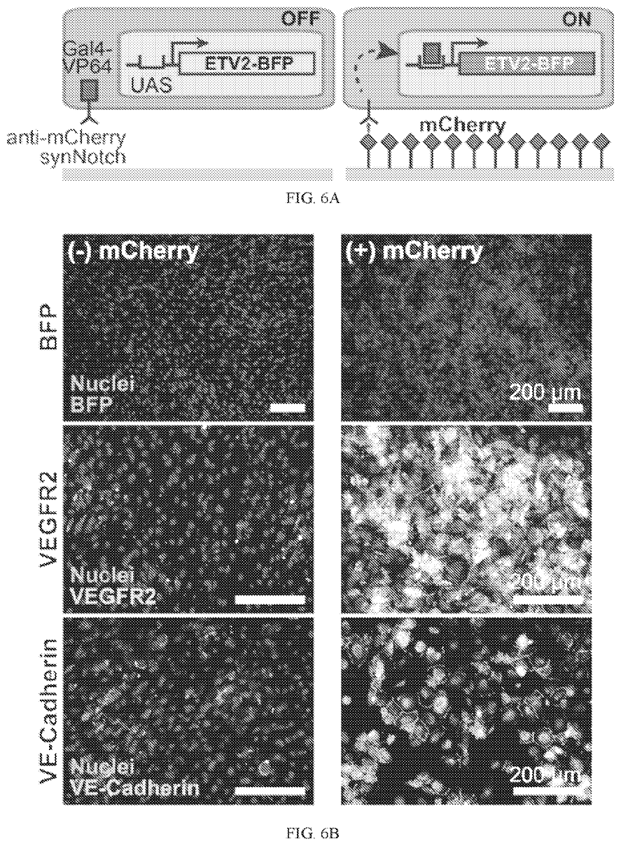

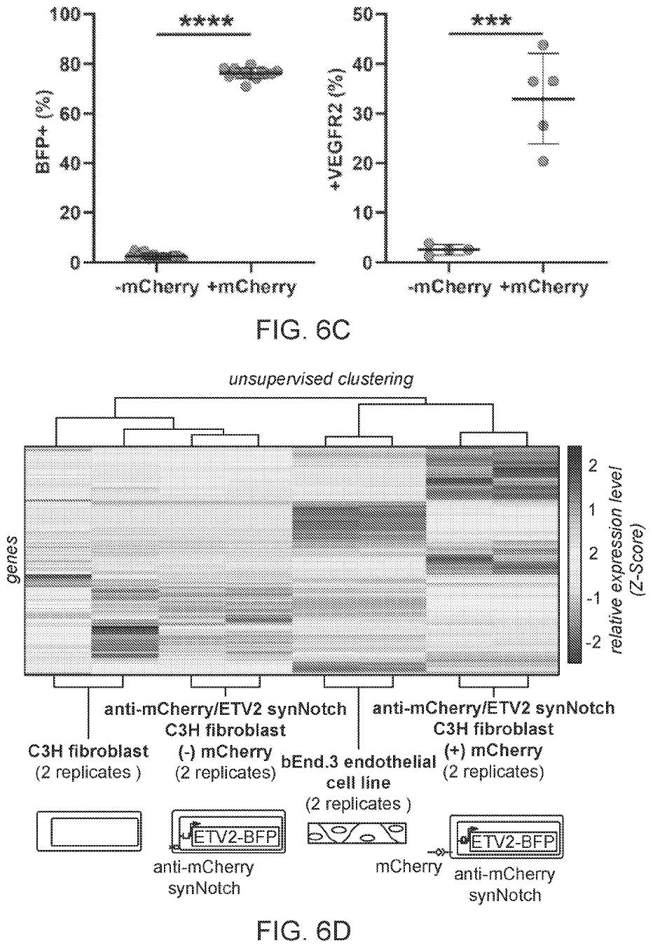

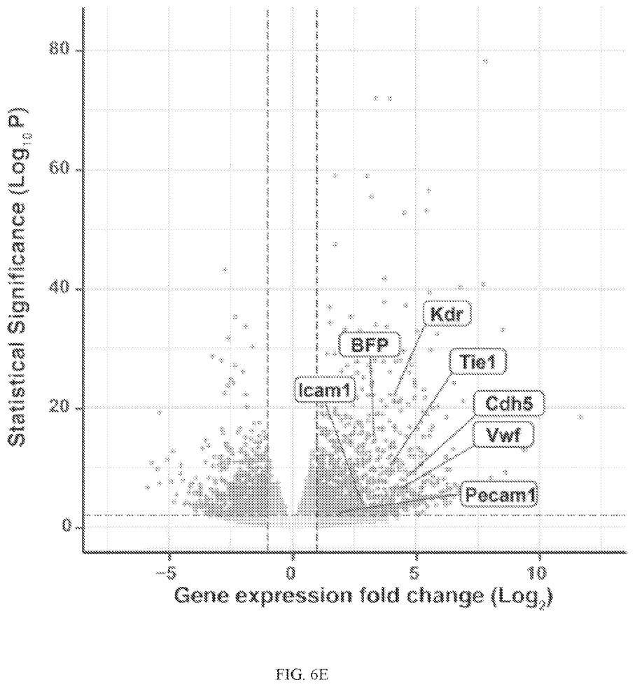

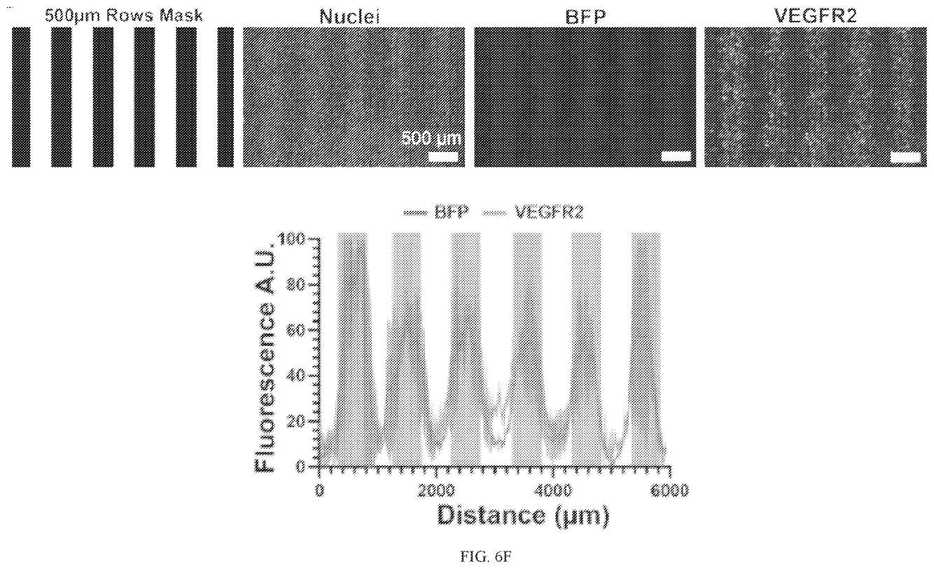

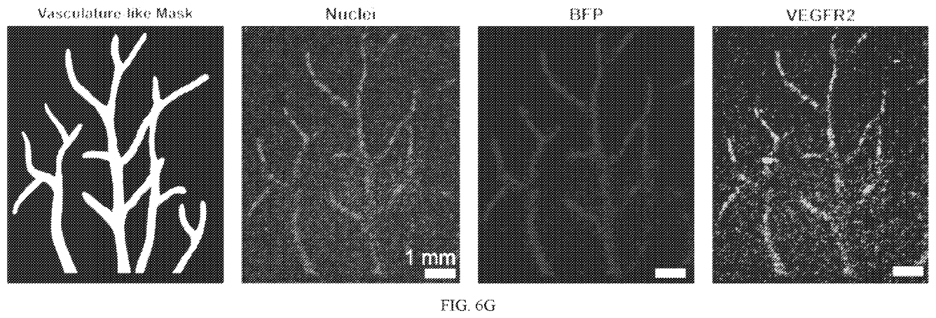

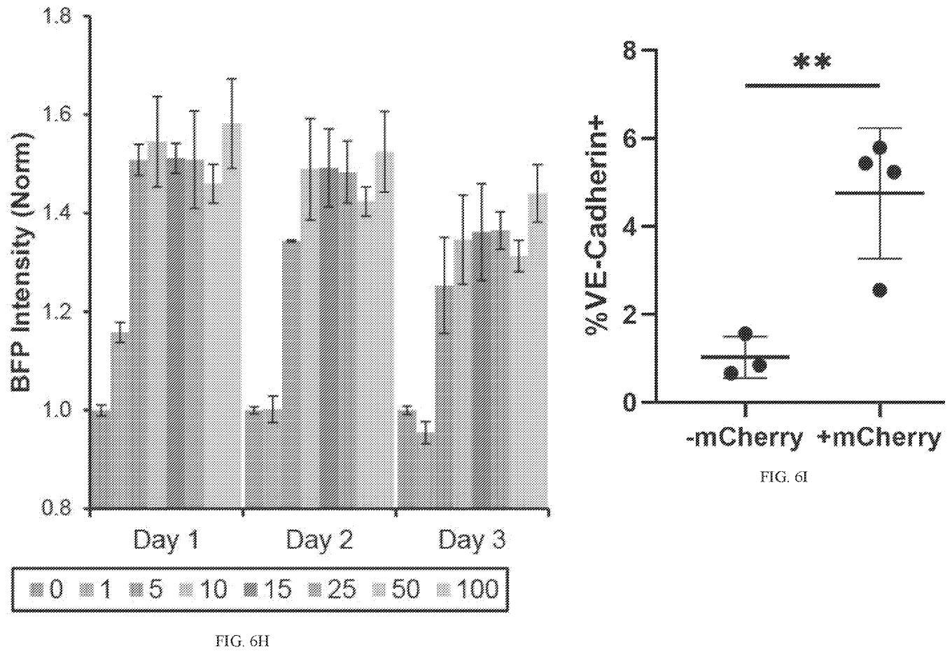

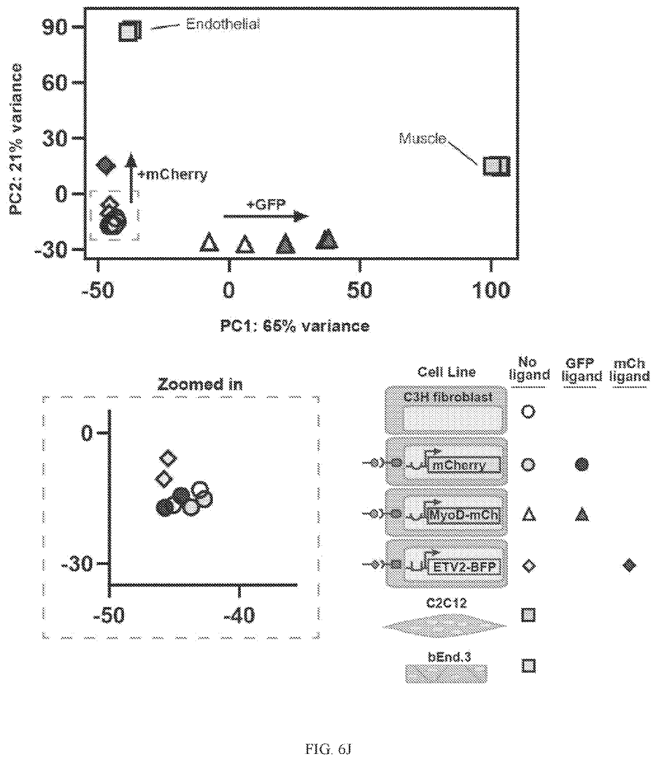

To exploit the modularity of this technology, we next tested if transdifferentiation to another cell fate could be activated by a similar approach. Due to the universal need for vascularization in engineered tissue constructs, including muscle, we focused on transdifferentiating fibroblasts into endothelial cells precursors, via doxycycline-inducible overexpression of the master transcription factors ETV2. Thus, we generated fibroblast receiver cells engineered with an anti-mCherry/Gal4 synNotch receptor that activates an ETV2-BFP cassette (FIG. 6A). We then passively adsorbed mCherry onto culture surfaces, cultured receiver cells on them for three days, and fixed and stained the cells for endothelial cell precursor markers. As shown in FIGS. 6B, 6C and 6H, the fibroblasts transdifferentiated to VEGFR2-positive endothelial precursors that also expressed VE-cadherin on their membrane. We also evaluated the differentiation trajectory by performing bulk RNA sequencing of receiver cells cultured on surfaces with or without mCherry, unmodified C3H fibroblasts, and Bend.3 endothelial cells as a positive control. We detected that culturing receiver cells on mCherry surfaces led to 3,022 differentially expressed genes. Receiver cells cultured on mCherry preferentially clustered with Bend.3 cells (FIG. 6D) and overexpressed endothelial-related genes, such as KDR and CDH5, compared to cells cultured on surfaces without mCherry (FIG. 6E). CDH5 (VE-Cadherin), a later-stage endothelial marker, was also detected at the protein level with flow cytometry (FIG. 6I). These data demonstrate that receiver cells expressing an anti-mCherry synNotch receptor that activates ETV transdifferentiated to endothelial cell precursors via mCherry adsorbed on a culture surface.

We also used Principal Component Analysis (PCA) to compare the transcriptome of unmodified C3H fibroblasts, cell type-specific positive control cells (C2C12 and Bend.3), and receiver cells that express fluorescent proteins, MyoD, or ETV2 in the presence or absence of their corresponding ligand. As shown in FIG. 6J, the presence of the respective receptor-ligand pair pushed receiver cells away from the unmodified C3H fibroblasts and towards the expected muscle or endothelial cell line. Receiver cells expressing fluorescent proteins also clustered with unmodified C3H cells in both the presence and absence of their respective ligand. Receiver cells expressing anti-GFP synNotch that activates MyoD also had a significant shift from the negative control cells towards C2C12 cells in the absence of GFP, indicating non-specific activation of the receptor. This was not observed for receiver cells expressing the anti-mCherry synNotch that activates ETV2.

To test if we can also control the geometry of transdifferentiation for the endothelial lineage, we generated uniformly adhesive surfaces and then microcontact printed mCherry in varying designs. We designed a pattern to replicate a branching network structure typical of vascular beds and showed the formation of a tissue consisting of activated cells in the corresponding pattern surrounded by a uniform layer of fibroblasts (FIG. 6D). Fibroblast receivers are activated by mCherry and express VEGFR2 based on the original ligand patterning, where we evaluated 500 μm rows and vascular branching pattern (FIG. 6F, 6G). Thus, similar to the myogenic synNotch cells, microcontact printed ligands can activate SynNotch-induced transdifferentiation to endothelial precursors with spatial control.

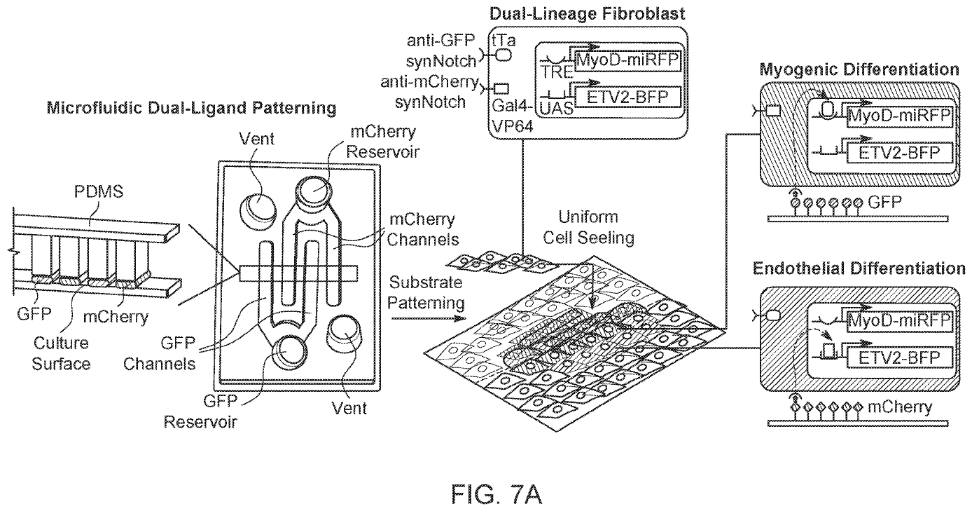

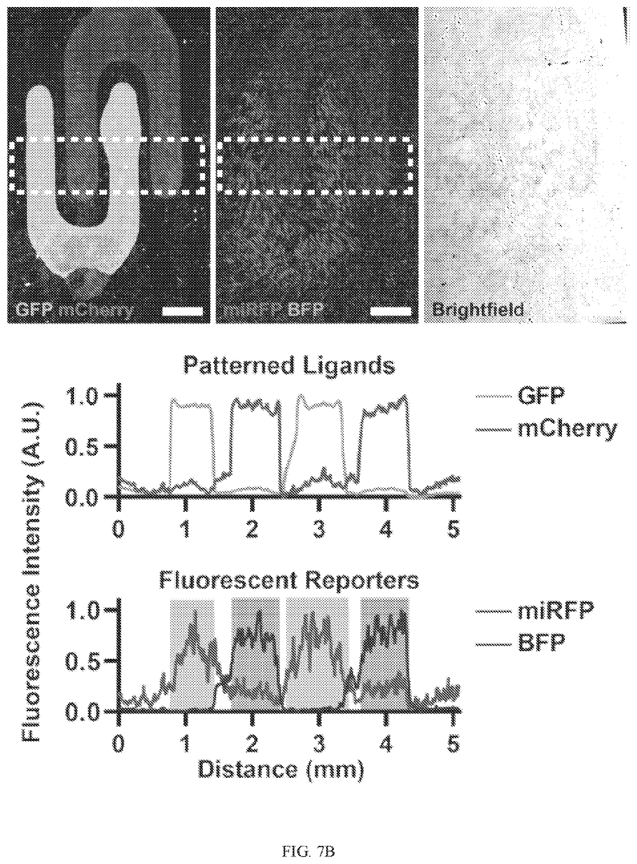

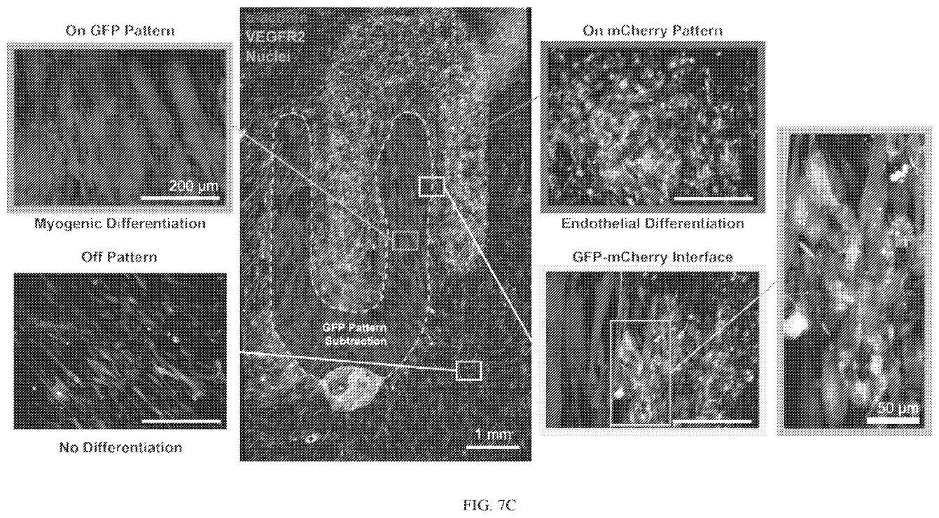

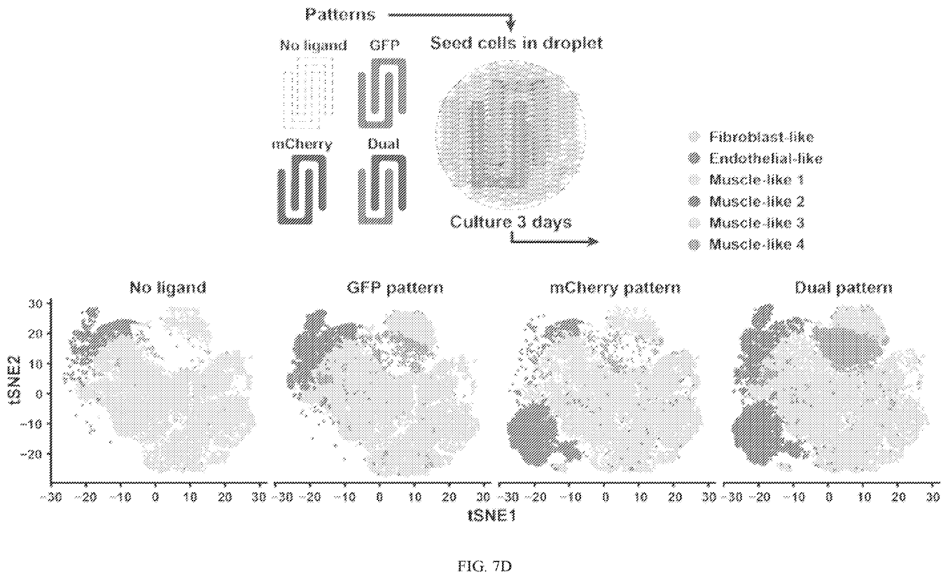

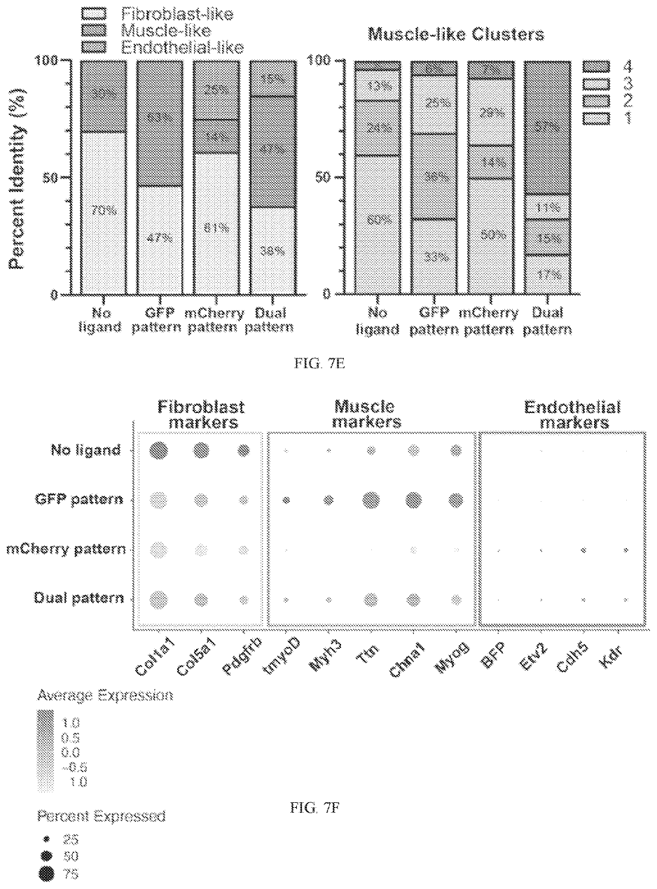

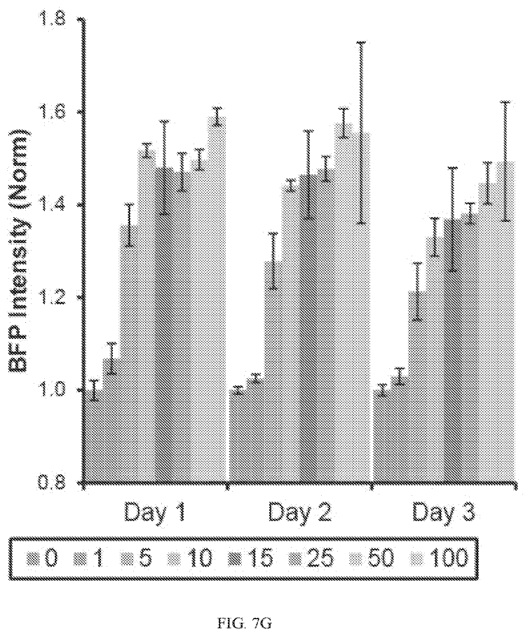

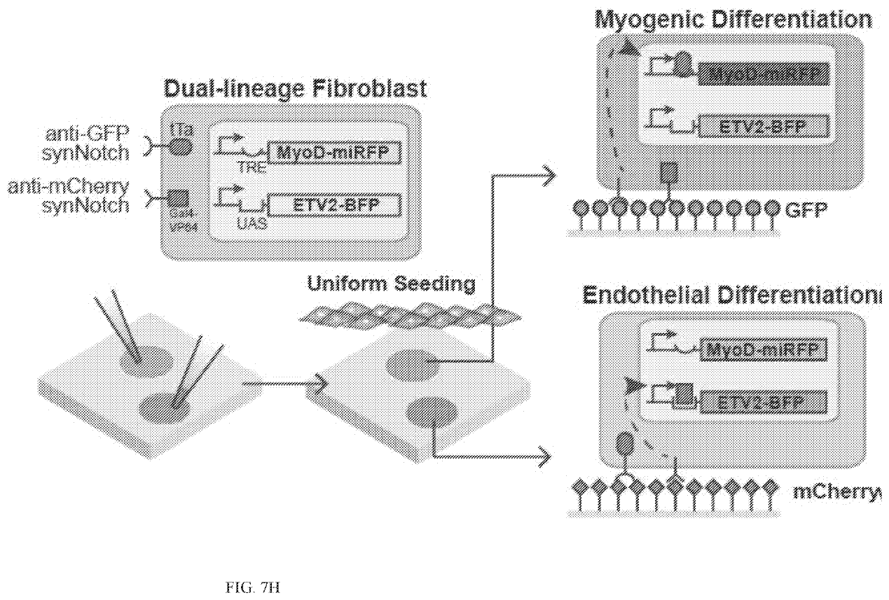

Finally, we asked if we could engineer a tissue construct in which multiple distinct cell fates are arranged in user-specified geometries. To do so, we first engineered a “dual-lineage” cell line with two synNotch pathways: an anti-GFP/tTA synNotch receptor that activates myoD-miRFP and an anti-mCherry/Gal4 synNotch that activates ETV2-BFP (FIG. 7A center). To test the functionality and orthogonality of these pathways, we cultured these cells on surfaces with a uniform coating of GFP or mCherry for three days and then stained for markers of differentiation. As shown in FIG. 7G, cells transdifferentiated to α-actinin-positive muscle precursor cells or VEGFR2-positive endothelial precursor cells, respectively. We evaluated the effects of culturing cells on both ligands, which would induce overexpression of both myoD and ETV2 in the same cells. In this case, it seemed that transdifferentiation to both pathways was impaired, as these cells did not differentiate towards skeletal muscle nor express endothelial cell markers. To prototype simple spatial activation, we used a micropipette to deposit droplets of GFP and mCherry in opposing corners of a culture surface (FIG. 7H). Dual lineage cells cultured on this surface activated the fluorescent protein reporters with expected spatial control, and displayed multinucleation in the GFP-coated region, indicating feasibility for spatial activation of differentiation.

Our next goal was to pattern multiple synNotch ligands onto a surface simultaneously and with spatial control. To do so, we controlled the distribution of multiple streams of liquids with an open capillary microfluidic device. Briefly, the intended fluid paths are created as shallow channels that are laterally open and adjacent to deep channels. Fluids preferentially travel along the shallow channels instead of the deep channels because of greater surface tension in shallow channels. We used this concept to design a microfluidic device for delivering solutions of GFP and mCherry as interdigiting 500 μm wide rows (FIG. 7A—left) and fabricated it by casting PDMS on 3-D printed inverse templates. Air vents and GFP and mCherry reservoirs were punched into the PDMS and the device was attached to a culture surface and loaded with GFP and mCherry solutions. After overnight incubation, the PDMS device was removed and remaining solutions were briefly air dried, leaving behind interdigitating rows of GFP and mCherry adsorbed on the surface (FIG. 7B). When dual-lineage cells were cultured on these surfaces, cells adhered uniformly to the entire surface and proceeded to transdifferentiate to myoblasts or endothelial cells in a pattern corresponding to the intended pattern of ligands (FIG. 7B, 7C). As shown in FIG. 7C, α-actinin-positive muscle precursor cells were confined to the GFP rows, VEGFR2-positive endothelial precursor cells were confined to the mCherry rows, and intermixing of these two cell types was observed at the interface between GFP and mCherry. Cells on the unpatterned regions remained fibroblasts.

To further evaluate the extent of dual-lineage transdifferentiation, we performed single-nuclei RNA sequencing on the dual-lineage cell line after three days of culture on substrates with no ligand, GFP-only rows, mCherry-only rows, and interdigitating GFP-mCherry rows, patterned using the technique shown in FIG. 7D. Due to the amount of culture area outside of the pattern, ligands activated approximately half of the total sequenced cells. T-Distributed Stochastic Neighbor Embedding (t-SNE) plot analysis identified twelve cell clusters based on gene expression profiles from all four conditions. We analyzed the clusters for signature genes and performed pathway analysis with DAVID, a web server for functional enrichment analysis and functional annotation of gene lists to assign each cluster to a putative cell type identity, resulting in seven fibroblast clusters, four muscle-like clusters, and one endothelial-like cluster. As shown in FIG. 7E, more cells were induced towards the myogenic lineage on GFP-only patterns and more cells were induced towards the endothelial lineage on mCherry-only patterns. On the dual GFP-mCherry pattern, both myogenic and endothelial clusters were detected (FIG. 7D, 7E). Selected marker gene analysis (FIG. 7F) showed that fibroblast marker genes were overall down-regulated on patterns with GFP and/or mCherry. Correspondingly, muscle-specific genes and endothelial-specific genes were over-expressed on patterns with GFP and/or mCherry, respectively (FIG. 7F). With this analysis, we also detected the expected expression of the transgenes (transgenic myoD and BFP). Interestingly, on the dual pattern, we observed more cells in the muscle-like 4 cluster compared to the other three patterns, indicating that this cell identity may be unique to co-differentiation. The four muscle clusters all express similar muscle marker genes, but at different relative levels. Pathway analysis of differentially expressed genes revealed the four muscle clusters differ in pathways related to cell cycle, ribosome, and differentiation, indicating that these four clusters may represent similar cells at slightly different phases of the cell cycle or stages of differentiation. Alternatively, co-differentiation may have unique impacts on cell phenotype. Thus, in summary, by activating synNotch receptors with microfabricated biomaterials, we induced a single population of fibroblasts to differentiate into a tissue with three distinct cell populations (skeletal muscle precursors, endothelial precursors, and fibroblasts) patterned in user-defined microscale geometries. Of note, these tissues were maintained in standard cell culture media, without the need for soluble differentiation factors or biophysical stimulation to drive cell fates. Overall, in this study, we engineered several material-to-cell signaling pathways to spatially activate user-defined genetic programs in multicellular systems. We achieved this by engineering cells with synNotch receptors to define cellular inputs and outputs while concurrently engineering materials to present synthetic ligands with different ranges of spatial control. The variety of materials for synthetic ligand presentation yields powerful and highly flexible tools for activating material-to cell pathways. Due to the functional modularity of synNotch receptors, material-activated pathways can theoretically be used to drive any number of transcriptional programs or differentiation pathways. These generalizable technologies are a new approach for dictating spatial patterning of gene expression in multicellular constructs, without the need for soluble differentiation factors.

Because of the highly powerful level of transcriptional control over cell behaviors in natural systems, many efforts in synthetic biology have focused on engineering sophisticated transcriptional circuits. In the area of stem cell and cell differentiation, genetic overexpression of master transcription factors has demonstrated robust control over cell differentiation. Initially, the effect of only a handful of master transcription factors on cell differentiation was known. However, more recently, approaches that collect entire organism transcription factor libraries have become available, making it feasible to induce multiple differentiation pathways with technologies (detail in Ng et al., Nature Biotechnology, 39, 510 (2021); Joung et al., Cell, 186, 209-229.e26 (2023)). Although optogenetic approaches have the potential for powerful spatiotemporal control over cell behaviors, optogenetic technologies require sophisticated light manipulation devices, which can be difficult to scale and have limited penetration into 3-D tissues, and have not yet demonstrated robust multi-cell fate control.

Herein we generated a new way to activate user-defined genetic programs via user-defined ligands presented by neighboring cells. Here, we advanced this technology to a new level by activating synNotch via multiple materials commonly used for tissue engineering. Importantly, we showed that this new approach can be used to define spatial patterns of not only gene expression, but also differentiation. To present synthetic ligands, we modified several different types of materials, each with trade-offs. By engineering cells to secrete fusions of synthetic ligands and the natural ECM protein fibronectin, ligands are presented in a natural ECM network comprising a diversity of endogenous macromolecules, which may enhance receiver cell adhesion and survival. However, spatial control is very coarse, as spatial ECM deposition by cells is not fully understood or controllable. Hydrogels are the most common class of materials for tissue engineering due to their high water content and multiple tunable properties, including stiffness, porosity, and composition. Thus, we developed versatile and modular methods for presenting synthetic ligands via hydrogels, by using relatively simple enzymatic reactions or click chemistry reactions to conjugate GFP or mCherry on the surface of gelatin or fibrinogen in 2D cultures or within bulk hydrogels for 3D cultures. Many modalities exist to conjugate and/or release proteins from hydrogels with spatial control (Qazi et al., Cell Stem Cell 29, 678-691, 2022), which can be integrated for use in the present system.

Bulk RNA sequencing and principal component analysis of single-lineage receiver cell lines on materials with single ligands validated that cells were transdifferentiating towards the intended myogenic or endothelial lineages. Different transcription factors likely have different activation amplitudes and dynamics and it is conceived to also identify appropriate signal-to-noise ratios for each specific application by, for example, generating synNotch receiver cells with different amounts of receptor and target gene constructs and assessing experimentally which combination works more efficiently for the transgene of interest.

In terms of heterogeneity, we observed bimodal and therefore incomplete activation of synNotch by ligands presented by materials, similar to other studies that have presented synNotch ligands from cells or other materials. Across all materials we tested, we found that synNotch activation reached a plateau in response to increasing ligand concentration, beyond which synNotch activation did not increase. Thus, we likely reached the saturation point of ligand presentation by the material and synNotch signaling itself seems to be the main factor limiting activation. We correspondingly observed differentiation efficiency being likely affected by a compounded effect of the heterogeneity of synNotch activation and the heterogeneity of transcription-factor-mediated differentiation. These are major limitations of synNotch but will continue to improve as the technology evolves.

In terms of dynamics, we found that different ligand-presenting materials yielded different temporal patterns of synNotch activation. For example, synNotch activation peaked at three days and then subsided when ligands were microcontact printed on PDMS, whereas activation was more sustained when synNotch was activated by ligands conjugated to 3-D hydrogels. This could be caused by differences in the conjugation of the ligands to the materials, such as the strength of the material-ligand bond or ligand orientation, and/or differences in ligand-receptor engagement and the activation of the synNotch receptor itself. The mechanism of transduction by synNotch receptors is thought to proceed similarly to endogenous Notch receptors: there, in the core regulatory region of endogenous Notch receptors, a pulling force is generated upon ligand binding, which exposes a protease cleavage site for a protease that is constitutively active in the membrane; this cleavage then liberates the intracellular domain which is a transcription co-activator. The mechanism of activation of synNotch by material-presented ligands may also differ from cell-presented ligands and may differ for different materials with various chemical and mechanical properties.

Another interesting result from our study is the impact of both ligands on dual receiver cells. For cells with two synNotch pathways that activate fluorescent reporters, both reporters were expressed in cells cultured on both ligands. However, for cells with two synNotch pathways that active myoD or ETV2 cultured on both ligands, both myogenic and endothelial differentiation programs were stunted. To achieve dual differentiation in the presence of two ligands, lineage bifurcation moduli (such as lateral-inhibition) or cross-inhibition to prevent the opposing lineage could lead to a salt-and-pepper or checkerboard pattern of differentiation in regions with both ligands. These types of approaches could also be combined with the MATRIX system described by Lee et al. in Biomaterials 297, 122099 (2023) to provide ligands at specific time points and achieve more advanced spatial and temporal control over differentiation, resulting in more complex tissue patterns.

To achieve greater spatial control, we microfabricated PDMS stamps and microfluidic devices to pattern synthetic ligands onto 2-D surfaces. By fabricating these components on 3-D printed templates instead of classical photolithography-based wafers, we achieved a wider range of pattern designs and more rapid prototyping capabilities, with the tradeoff that spatial resolution was preferably confined to 100 μm or above. To pattern synthetic ligands at sub-cellular spatial resolution, photolithography would still be required. The two PDMS-based patterning technologies that we used also have tradeoffs. Microcontact printing can generate essentially any geometrical pattern (including isolated islands) but cannot precisely register multiple ligands since each stamp must be positioned manually. Conversely, registering the placement of multiple ligands is possible with a microfluidic device, but pattern geometries are likely limited to continuous channels connected to a reservoir. Thus, these constraints must be considered when choosing a patterning modality. Together with other approaches, such as MATRIX, these new approaches expand the library of engineered biomaterials that activate synNotch.

Our most sophisticated tissue construct comprised interdigitating rows of skeletal muscle and endothelial cells, with some intermingling of the cells at the interface. Importantly, the skeletal muscle cells and endothelial cells were co-transdifferentiated from a single population of fibroblasts. This approach is in contrast to conventional tissue engineering techniques, which usually differentiate individual cell types in isolation and then combine them. Our approach may better mimic natural tissue morphogenesis, where multiple cell fates emerge simultaneously from a uniform cell population. Studies have also shown that supporting cells, such as endothelial cells, improve the maturation of human induced pluripotent stem cell-derived cardiomyocytes, and that co-differentiation of different lineages concurrently more closely recapitulate the conditions occurring during embryonic development. An interesting hypothesis to explore with our technology is whether co-differentiation of supporting cells (e.g., endothelial cells) adjacent to parenchymal cells (e.g., muscle cells) has additional benefits for phenotypic maturity. For example, our single-nuclei sequencing revealed one muscle-like cluster that was overrepresented on the dual-ligand pattern compared to the GFP-only pattern. There are many potential explanations for this, such as: (i) these muscle-like cells were uniquely influenced by the presence of the co-differentiating endothelial cells; (ii) these muscle-like cells were located at the GFP-mCherry boundary and thus were activated predominantly by GFP but also by mCherry to a lower extent; and/or (iii) these muscle-like cells were coincidentally captured at a unique stage of cell cycle or differentiation but are otherwise similar to the other muscle-like clusters. More complete lineage conversion to mature cell types may be seen beyond 3 days for differentiation and potentially supplementation with soluble differentiation factors for some cell types.

We also conceive methods combining bioprinting and synNotch technologies by, for example, bioprinting hydrogels that are functionalized with synNotch ligands, such as the gelMA and fibrinogen that we synthesized in the Examples herein. Optogenetic technologies may also be integrated to add more temporal control of cell phenotype. Overall, the ongoing integration of synthetic biology, biomaterials, and microfabrication technologies will further advance the capabilities for tissue engineering.

We further conceive that our approach for activating synthetic pathways for transdifferentiation by a material can be combined with other technologies such as organoids. To generate organoids, stem cells are exposed to natural ligands that orchestrate their self-organization into complex cellular arrangements. However, although cellular complexity at the microscale in organoids is remarkably similar to endogenous organs, users lack geometric control over the arrangement of cells at higher levels, leading to tissue constructs that are largely heterogeneous and poorly reproducible with un-natural architectural features. Combining synthetic biology and organoids is a recognized frontier of the field and synNotch-mediated spatial patterning technologies could represent a step in the direction of ultimate user-control of cell behaviors across multiple spatial scales for engineering in vitro multicellular systems.

### Stem Cell Applications

For example, we demonstrated that anti-GFP synNotch receptors engineered in mES (mouse embryonic stem) cells can be activated via plate-presented ligands.

On tissue culture wells, we cultured mESC and they grew; and we could pattern the GFP using one of the ways described above. In one example, a 100-200 ug/mL at 5-15 uL droplet of GFP is added for a final surface concentration of 12-15 ug/cm²; and the GFP solution is dried for 1-2 hours before seeding with cells.

Gelatin coated wells may also suit ES cells, and patterning can be realized as well. For example, 1) microwells or wells are with gelatin, e.g., 1× gelatin to coat an entire well surface and incubated in 37° C. incubator for 30 min, and excess liquid is aspirated;
2a) Dry the gelatin surface, e.g., leave well lid open in the back of the biosafety cabinet with fan on to facilitate drying. In doing so, the GFP droplet does not spread across the entire surface of wet gelatin. Drying may take 1-3 hours depending on the airflow.
2b) Or an entire surface is to be coated with GFP solution, no drying is required; and appropriate amount of GFP to reach 12-15 ug/cm² surface should be added in step 1).
3a) Allow GFP to dry. This dry time will vary depending on how much volume is added. Dilute (e.g., water) from the GFP solution should be evaporated as much as possible so that substantially only the GFP is deposited onto the surface. This would also reduce any undesired diffusion, mixing, or ‘leakage’ of GFP patterning when liquid media (with cells) are seeded. When GFP is dried, cells may be seeded.
3b) If GFP is applied to a certain area and not the whole well, the DMEM media should be gently pipetted with 10% FBS to cover the entire surface and incubate at 4° C. for ~1 hour before seeding. This prevents the GFP from smearing. Excess DMEM/FBS solution should be removed before seeding cells.
3c) If GFP is to be applied uniformly throughout the well/surface, the well may be gently washed with PBS before seeding with cells.

After seeding, transgene expression (e.g., mCherry reporter) is visible starting at 24 h post seeding via FACS and microscopy.

### Techniques, Materials, and Equipment

### Genetic Constructs Design:

Fibronectin-GFP plasmids were generated from PiggyBac backbone and FN-YPET (Addgene #65421). GFP and mCherry-responsive synNotch construction: pHR_SFFV_myc-LaG17_synNotch_TetRVP64 (Addgene plasmid #79128) and pHR_EF1a_flag-LaM4_synNotch_Gal4-VP64, built from pHR_EF1a_flag-LaM4_synNotch_TetRVP64 (Addgene plasmid #162237) and HR_pGK_LaG17_synNotch_Gal4VP64 (Addgene plasmid #79127). The response-element plasmids pHR_TRE_MyoD-P2A-mCherry, pHR_TRE_MyoD-P2A-miRFP703_PGK_PuromycinR, and pHR_UAS_ETV2-P2A-tBFP_PGK_HygromycinR (with and without transcription factor) were generated from pHR_TRE, pHR_5× Gal4 UAS (Addgene plasmid #79119), mouse MyoD (NP_034996.2), and mouse ETV2 (NP_031985.2). All constructs were cloned via In-Fusion HD Cloning (Takara Bio).

Amino Acid Sequence of an Exemplary Anti-GFP synNotch tTA:

(SEQ ID NO: 3)
MALPVTALLLPLALLLHAARPEQKLISEEDLMADVQLVESGGGLVQAGG
SLRLSCAASGRTISMAAMSWFRQAPGKEREFVAGISRSAGSAVHADSVK
GRFTISRDNTKNTLYLQMNSLKAEDTAVYYCAVRTSGFFGSIPRTGTAF
DYWGQGTQVTVSILDYSFTGGAGRDIPPPQIEEACELPECQVDAGNKVC
NLQCNNHACGWDGGDCSLNFNDPWKNCTQSLQCWKYFSDGHCDSQCNSA
GCLFDGFDCQLTEGQCNPLYDQYCKDHFSDGHCDQGCNSAECEWDGLDC
AEHVPERLAAGTLVLVVLLPPDQLRNNSFHFLRELSHVLHTNVVFKRDA
QGQQMIFPYYGHEEELRKHPIKRSTVGWATSSLLPGTSGGRQRRELDPM
DIRGSIVYLEIDNRQCVQSSSQCFQSATDVAAFLGALASLGSLNIPYKI
EAVKSEPVEPPLPSQLHLMYVAAAAFVLLFFVGCGVLLSRKRRRQLCIQ
KLMSRLDKSKVINSALELLNEVGIEGLTTRKLAQKLGVEQPTLYWHVKN
KRALLDALAIEMLDRHHTHFCPLEGESWQDFLRNNAKSFRCALLSHRDG
AKVHLGTRPTEKQYETLENQLAFLCQQGFSLENALYALSAVGHFTLGCV
LEDQEHQVAKEERETPTTDSMPPLLRQAIELFDHQGAEPAFLFGLELII
CGLEKQLKCESGGPADALDDFDLDMLPADALDDFDLDMLPADALDDFDL
DMLPG.

The extracellular domain or a GFP-specific targeting domain has an amino acid sequence of:

(SEQ ID NO: 4)
MALPVTALLLPLALLLHAARPEQKLISEEDLMADVQLVESGGGLVQAGG
SLRLSCAASGRTISMAAMSWFRQAPGKEREFVAGISRSAGSAVHADSVK
GRFTISRDNTKNTLYLQMNSLKAEDTAVYYCAVRTSGFFGSIPRTGTAF
DYWGQGTQVTVS.

The Notch regulatory region (comprising a ligand-inducible proteolytic cleavage site) has an amino acid sequence of:

(SEQ ID NO: 5)
PPQIEEACELPECQVDAGNKVCNLQCNNHACGWDGGDCSLNFNDPWKNC
TQSLQCWKYFSDGHCDSQCNSAGCLFDGFDCQLTEGQCNPLYDQYCKDH
FSDGHCDQGCNSAECEWDGLDCAEHVPERLAAGTLVLVVLLPPDQLRNN
SFHFLRELSHVLHTNVVFKRDAQGQQMIFPYYGHEEELRKHPIKRSTVG
WATSSLLPGTSGGRQRRELDPMDIRGSIVYLEIDNRQCVQSSSQCFQSA
TDVAAFLGALASLGSLNIPYKIEAVKSEPVEPPLPSQLHLMYVAAAAFV
LLFFVGCGVLLS.

The intracellular domain (comprising a transcriptional activator) has an amino acid sequence of:

(SEQ ID NO: 6)
MSRLDKSKVINSALELLNEVGIEGLTTRKLAQKLGVEQPTLYWHVKNKR
ALLDALAIEMLDRHHTHFCPLEGESWQDFLRNNAKSFRCALLSHRDGAK
VHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGGPADALDDFDLDMLPADALDDFDLDMLPADALDDFDLDM
LPG*;

A linker between the GFP-specific targeting domain and the Notch regulatory domain has an amino acid sequence of ILDYSFTGGAGRDIP (SEQ ID NO:7); and
a linker between the Notch regulatory domain and the tTA-advanced has an amino acid sequence of: RKRRRQLCIQKL (SEQ ID NO:8).

### Amino Acid Sequence of an Exemplary Anti-mCherry SynNotch Gal4:

(SEQ ID NO: 12)
MALPVTALLLPLALLLHAARPDYKDDDDKMAQVQLVESGGSLVQPGGSL
RLSCAASGRFAESSSMGWFRQAPGKEREFVAAISWSGGATNYADSAKGR
FTLSRDNTKNTVYLQMNSLKPDDTAVYYCAANLGNYISSNQRLYGYWGQ
GTQVTVSSPFTILDYSFTGGAGRDIPPPOIEEACELPECOVDAGNKVCN
LOCNNHACGWDGGDCSLNFNDPWKNCTOSLQCWKYFSDGHCDSQCNSAG
CLFDGFDCQLTEGQCNPLYDQYCKDHFSDGHCDQGCNSAECEWDGLDCA
EHVPERLAAGTLVLVVLLPPDQLRNNSFHFLRELSHVLHTNVVFKRDAQ
GQQMIFPYYGHEEELRKHPIKRSTVGWATSSLLPGTSGGRORRELDPMD
IRGSIVYLEIDNRQCVOSSSQCFQSATDVAAFLGALASLGSLNIPYKIE
AVKSEPVEPPLPSQLHLMYVAAAAFVLLFFVGCGVLLSRKRRRMKLLSS
IEQACDICRLKKLKCSKEKPKCAKCLKNNWECRYSPKTKRSPLTRAHLT
EVESRLERLEQLFLLIFPREDLDMILKMDSLQDIKALLTGLFVQDNVNK
DAVTDRLASVETDMPLTLRQHRISATSSSEESSNKGQRQLTVSAAAGGS
GGSGGSDALDDFDLDMLGSDALDDFDLDMLGSDALDDFDLDMLGSDALD
DFDLDMLGS.

The amino acids in italics in the sequence of the exemplary anti-mCherry SynNotch Gal4 is the Notch regulatory region, SEQ ID NO:5; a linker of SEQ ID NO:7 is included to the N-terminus of the fragment having a sequence of SEQ ID NO:5; and amino acid residues 1-158 of SEQ ID NO:12 includes the mCherry-specific targeting domain; and amino acid residues 429-695 of SEQ ID NO:12 includes an intracellular domain.

### Lentivirus Production:

Lentivirus was produced by cotransfecting pHR cloned plasmids with vectors encoding packaging proteins (psPAX2, pVSVG) using Lipofectamine LTX (ThermoFisher) into 70-80% confluent HEK-293T cells within 6-well plates. Viral supernatants were collected 2-3 days after transfection, sterile filtered with 0.45 μm PES (Genesee Scientific), and used directly or 10× concentrated using LentiX Concentrator (Takara Bio) following manufacturers instructions prior to adding to cell lines.

### Cell Culture:

L929 mouse fibroblast cells (ATCC #CCL-1), HEK293 cells (Takara 632180), C3H/10T1/2 Clone 8 (ATCC #CCL-226), and NIH/3T3 (ATCC #CRL-1658) were cultured in DMEM (ThermoFisher) supplemented with 10% Fetal Bovine Serum (ThermoFisher) and 100U/mL penicillin/streptomycin (ThermoFisher). Cultures were maintained in a 37° C. incubator with 5% CO₂ and relative humidity (VWR).

### Cell Line Engineering:

For generation of 3T3 fibroblasts expression FN-GFP, 20,000 3T3 cells were seeded in a 12-well plate. The following day cells were transfected with 1 ug FN-eGFP PiggyBac plasmid using 2.5 μL Lipofectamine LTX with 1 μL Plus reagent diluted in 100 μL OptiMEM. Transfected cells were selected using 2 μg/mL Puromycin. Additionally, the established line was transduced with lentivirus encoding the expression of constitutive H2B-miRFP703.

For viral transduction, 20-50 μL concentrated (or equivalent non-concentrated) viral supernatant(s) were added to 5-10×10⁴ suspended cells supplemented with 10 μg/mL polybrene (Sigma), then transferred into a 12-well plate for 2-3 days before changing to fresh media. Following transduction, all applicable cell lines were selected using Puromycin (L929—10 μg/mL, C3H—1 μg/mL, NIH3T3—2 μg/mL, ThermoFisher) and Hygromycin B (L929, C3H—400 μg/mL, MedChem Express) for the expression of transgenes. Cells were sorted for the coexpression of each component via fluorescence-activated cell sorting on a FACS ARIA II (Beckton-Dickinson) by staining with appropriate fluorescently tagged anti-Myc and anti-Flag antibody for 30 minutes at 4° C. (Cell Signaling Technologies) or expression of the transgenes. A bulk-sorted polyclonal population of engineered cells were used for experiments, unless otherwise noted. For single-cell clonal populations, single cells were sorted individually into 96-well plates from selected and stained populations using a FACS ARIA II.

GFP and mCherry Production:

GFP, mCherry, and GFP-LACE (pET28-His6-GFP-C-LACE, gift from Jeffrey Bode Addgene plasmid #133913) were purified as an N-terminal hexahistidine fusion protein. To express GFP, BL21(T1R) E. coli cells were grown to an optical density of 0.5 from an overnight-grown glycerol stock, chilled to 25° C., induced with 1 mM IPTG and allowed to express for 5 hours. To express mCherry, BL21-AI E. coli (Thermo Fisher) were transformed with mCherry-pBAD (gift from Michael Davidson & Nathan Shaner & Roger Tsien, Addgene plasmid #54630), grown to an optical density of 0.6 from an overnight-grown glycerol stock, induced with 0.04% w/v L-Arabinose (Sigma), and allowed to express for 5 hours (dergipark.org.tr/en/pub/iarej/issue/44303/429547). The proteins were purified by NEBExpress Ni Spin Columns (New England Biolabs) following manufacturer's instructions, dialyzed against 1×PBS overnight at 4° C., sterile filtered, and frozen at −80° C. until use.

### Microparticle-Conjugation and Activation:

Carboxylated magnetic polystyrene microparticles (Magsphere, MCA5UM) were first washed in 0.1M MES (pH 5.8), activated with 250 mM 1-Ethyl-3-(3-dimethylaminopropyl)carbodiimide (EDC, Sigma)/N-Hydroxysuccinimide (NHS, Sigma) in MES for 15 minutes, then washed two times with PBS (pH 7.4). Particles were then incubated in varying concentrations of GFP in PBS (0-1000 μg/mL) overnight at 4° C. with inversion mixing. All washing steps used EasySep Magnet (Stemcell Technologies) or centrifugation (8000g for 5 minutes) to change solutions. Particle concentration was determined using hemocytometer and directly loaded into cell suspensions (10 particles per cell) prior to seeding L929 anti-GFP synNotch receiver cells 25,000 cells/cm² directly into wells or gelatin-coated coverslips. 24 hours after seeding, samples were imaged directly using Zeiss Axio Observer Z1 or stained with NucBlue and fixed with 4% PFA for 10 minutes. Individual cell mCherry intensity was quantified on ImageJ using nuclear segmentation from 5 images per sample. Gaussian blur, thresholding, watershed, and analyze particle functions were applied to the nuclei and mCherry channel and to create individual selections for total cells and activated cells, respectively. The mCherry mask was applied to the corresponding mCherry image to measure the average fluorescence intensity within each activated cell. Percent activation was determined by the number of mCherry positive cells divided by the total number of nuclei. Activated mCherry intensity was calculated by averaging the mCherry intensity for cells above the defined threshold.

### Fibronectin-GFP Activation:

For local activation, L929 anti-GFP synNotch receiver cells were seeded with 3T3 cells expressing FN-eGFP and nuclear localized miRFP703 in a ratio of 50:1 to a total of 5×10⁴ cells per well on a 8-well slide (Ibidi). After 3 days, cells were imaged on a Zeiss LSM780. For the titration experiments, cells were seeded in 8-well ibidi slides coated with 0.1% gelatin to a total of 3×10⁴ parental 3T3 cells and FN-GFP sender cells in following ratios 1:0, 50:1, 5:1, 2:1, 1:1 and 0:1 (Parental: FN-GFP). Following 8-10 days of culture, decellularization was performed. Briefly, cell-laden ECM was washed with PBS, wash buffers, and lysis buffer containing NP-40 for up to two hours to remove cellular debris. Removal of nuclear debris was confirmed by Hoechst staining prior to decellularization, which was used to monitor decellularization quality during the lysis phase of the protocol. Decellularized ECM was used immediately or stored at 4° C. until use. 5-10×10⁴ L929 anti-GFP-synNotch receiver cells were seeded onto the decellularized matrices. After 2 days cells were imaged on a Zeiss LSM780 or Keyence BZ-X and on the same day analyzed by FACS on a Thermo Fisher Attune. For other co-culture experiments, L929 anti-GFP (or anti-mCherry) synNotch receiver cells were seeded at a 1:1 ratio with FN-GFP or FN-mCherry senders. Activation of engineered cells was imaged using Keyence BZ-X or fixed, stained for fibronectin (primary O/N 4° C., secondary 1 hour RT), and imaged on a Zeiss LSM780.

### Gelatin Hydrogel Surface Conjugation:

Gelatin hydrogels were fabricated. Briefly, a 30W Epilog Mini 24 laser engraver (100% speed, 25% power, 2500 Hz) was used to cut a 150-mm polystyrene dish into 260-mm² hexagons. Each hexagon was masked with tape, and an inner circle was cut (18% speed, 6% power, 2500 Hz) and removed, exposing a polystyrene surface which was then treated with plasma (Harrick Plasma) for 10 minutes to improve gelatin adherence to polystyrene. Equal volumes of a 20% porcine gelatin solution (Sigma) and 8% MTG (Ajinomoto) solution were mixed and 200 μl were added to each coverslip. Flat or 10×10 μm micromolded PDMS stamps were immediately applied to shape surface topography. After an overnight incubation to solidify, the hydrogels were rehydrated in water, and the stamp was removed. Coverslips were stored in PBS at 4° C. until cell seeding.

PDMS stamps with 10×10 μm grooves of 2 μm height were fabricated with standard photolithograpy and soft lithography techniques. Flat PDMS was used as a control substrate with no topography. A 1:1 ratio of GFP (500 μg/ml, 200 μg/ml, and 20 μg/ml) and MTG (8% w/v) solution were added to a parafilm surface, and the gelatin coverslip was inverted onto the GFP-MTG droplet for 10 minutes. The coverslips were then incubated for 1 hour at 37° C. Following incubation, the coverslips were washed 3 times with warm PBS.

L929 anti-GFP synNotch receiver cells were seeded at a density of 350,000 cells per coverslip and cultured for 72 hours. To detach the cells from the gelatin for flow cytometry analysis, the gelatin hydrogels were minced with a sterile X-acto knife and incubated in a 4 mg/ml collagenase IV solution for 45 minutes at 37° C. Digested gelatin was then filtered with a 40 μm cell strainer.

C3H anti-GFP synNotch MyoD expressing cells were seeded at a density of 500,000 cells per coverslip, and cultured for 4 days. Coverslips were washed three times with warm PBS, fixed with ice-cold methanol and immunostained with mouse α-actinin primary antibody (Sigma, A7811) at 1:200 dilution for two hours. Coverslips were then stained with the secondary antibody goat anti-mouse conjugated to Alexa Fluor 546 and 4′,6-diamidino-2-phenylindole (DAPI) at 1:200 dilutions for 2 hours. ProLong gold antifade mountant (Thermofisher) was used to mount cells on glass coverslips.

Myotube count was performed in ImageJ through size and intensity thresholding of the α-actinin signal. The total number of cells per image was calculated by size and intensity thresholding of the DAPI signal. Myogenic index was determined by dividing the number of co-localized nuclei within the α-actinin signal by the total number of nuclei in the field of view. The Orientation Order Parameter of both the myotubes and nuclei were determined by first analyzing images of the α-actinin and DAPI signal, respectively, using the OrientationJ Distribution plugin in ImageJ. This plugin was used to generate a histogram with the number of pixels locally oriented along every angle at 0.5 degree increments. This histogram was then analyzed using MATLAB code to calculate the Orientation Order Parameter, which ranges from 0 for completely randomized systems to 1 for perfectly aligned systems.

### 3D Ligand Conjugation:

GelMA Synthesis: Porcine Gelatin (175G Bloom, Sigma G2625) was dissolved at 10g in 100 mL in 0.25M carbonate-bicarbonate buffer (pH 9) at 50° C. under argon. 0.4 mL methacrylic anhydride (Sigma 276685) was added dropwise with stirring (500 rpm) and reacted for 3 hours at 50° C. The reaction was then cooled to 40° C., adjusted with 6M HCl to pH 7.4, transferred to 12-14 kDa cutoff dialysis tubing (Fisher Scientific), dialyzed for 3 days at 40° C. against 4L deionized water (changed twice daily), and lyophilized. GelMA was stored at −80° C. until use. Degree of methacrylation was calculated using ¹H-NMR compared to unmodified gelatin.

GelMA was dissolved at 1% w/v in PBS at 37° C. Methyltetrazine (mTz)-PEG5-NHS Ester (Click Chemistry Tools) was first dissolved at 8.8 mM in DMSO and added to GelMA solution dropwise with stirring to 0.88 mM final concentration. Mixture was reacted at 37° C. overnight, transferred to 12-14 kDa cutoff dialysis tubing (Fisher Scientific), and dialyzed for 3 days at 40° C. against 4L deionized water (changed twice daily), and lyophilized. Substitution was verified using ¹H-NMR compared to unmodified GelMA and used to estimate percent substitution of methacrylate and methyltetrazine. Ligands (GFP and mCherry) were modified using trans-Cyclooctene (TCO)-PEG4-NHS Ester (Click Chemistry Tools) to generate GFP-TCO and mCherry-TCO. TCO-PEG4-NHS Ester was dissolved at 10 mM in DMSO, added at a 20-molar excess to GFP and mCherry in PBS (1-3 mg/mL), and reacted for 1 hour at room temperature in Eppendorf tubes with orbital rocking. Following, mixture was purified using Zeba Spin Desalting Columns (7k MWCO, ThermoFisher) following manufacturer instructions, sterile filtered, and aliquoted to 1 mg/mL and stored at −80° C. Reactivity was verified by reacting GelMA-mTz at 1% w/v and GFP-TCO at 100 μg/mL for one hour at 37° C., running on 4-20% SDS-PAGE gel (Bio-Rad and Coomassie blue staining (Invitrogen).

### Cell Encapsulation and Activation:

GelMA-mTz at 1% w/v was reacted with GFP-TCO at 50-100 μg/mL for one hour at 37° C. Following, 18% w/v GelMA solution and freshly prepared 25 mg/mL Lithium phenyl-2,4,6-trimethylbenzoylphosphinate (LAP) was added to a final concentration of 5-10% w/v GelMA, 0-100 μg/mL GFP-TCO, and 0.25% LAP. Engineered L929 or C3H cell lines were resuspended in this solution at 5-10×10⁶ cells/mL. An array of 20 μL droplets of cell-laden hydrogel solutions were pipetted between two 25×75 mm glass slides that were thrice treated with GelSlick (Lonza) separated by 3D-printed 400 μm insert. Gels were crosslinked for 90-180s at 25 mW/cm² using an Omnicure2000 and collimating adapter (Excelitas) and transferred to individual wells with DMEM complete. Following 30 minute incubation at 37° C., media was exchanged to fresh DMEM complete. Cell-laden hydrogels were cultured up to 14 days with media changes every 2 days. In the hydrogel patterning experiment, cell-laden hydrogel solutions containing 0 or 50 μg/mL GFP were pipetted in proximity to each other to initiate limited contact immediately prior to crosslinking. This process led to a stable biphasic gel that was treated similarly as described above. Samples were imaged live with Zeiss Axio Observer Z1 or Zeiss LSM880 confocal microscope. Individual cell mCherry intensity was quantified on ImageJ using constitutive BFP-signal to segment individual cells. Gaussian blur, thresholding, watershed, and analyze particle functions were applied to the BFP channel to create individual selections for each cell. This mask was applied to the corresponding mCherry image to measure the average fluorescence intensity within each cell. Percent activation was determined by the number of BFP+ cells that had an mCherry intensity higher than a defined threshold. For spatial activation within 3D, line plots were quantified using the “Plot Profile” feature on inverted microscope images in ImageJ and normalized across the entire time course. Evaluation of cell viability was performed using Live/Dead Viability/Cytotoxicity Kit (ThermoFisher) following manufacturer's instructions. Briefly, cell-laden gels were incubated in a 2 μM calcein AM and 4 μM EthD-1 solution in PBS for 45 minutes prior to imaging on a Zeiss LSM880.

In the co-culture encapsulation experiment, GFP-sender fibroblasts and anti-GFP synNotch receiver cells were co-encapsulated within 5% GelMA hydrogels at a fixed total cell concentration of 20×10⁶/mL. The ratio of each cell type was varied. Hydrogels were imaged on an LSM880 and the percent mCherry expression was evaluated using image analysis, where the BFP-signal was used to segment individual receiver cells.

Fibrinogen (Sigma) was dissolved at 5 mg/mL in warmed PBS. Methyltetrazine (mTz)-PEG5-NHS Ester (Click Chemistry Tools) was first dissolved at 20 mM in DMSO and added to the Fibrinogen solution dropwise to a 0.16 mM final concentration. Solution was incubated at room temperature with manual rocking every 10 minutes for one hour, transferred to 12-14 kDa cutoff dialysis tubing (Fisher Scientific), and dialyzed overnight at 4° C. against 12L deionized water, and lyophilized. Resulting Fibrinogen-mTz was stored at −80° C. until use. For generation of Fibrinogen-mCherry and cell encapsulation, Fibrinogen-mTz was dissolved at 20 mg/mL in PBS and incubated with 160 μg/mL mCherry-TCO in PBS for one hour at 37° C. DMEM was added to bring to a final 10 mg/mL Fibrinogen+100 μg/mL mCherry and used to resuspend cell pellet at 5×10⁶ anti-mCherry synNotch receiver cells/mL. Immediately prior, 0.2U Thrombin/mg Fibrinogen was added to the solution and samples were allowed to gel for 10 minutes at 37° C. Following a 15 minute wash with DMEM to remove unbound mCherry, samples were transferred to the incubator for culture. Following 24 hours of culture, HSC NuclearMask Deep Red was added for one hour at 37° C. to visualize nuclei before imaging on Zeiss Axio Observer Z1.

### Microcontact Printing:

For all surfaces except FIG. 5L, 18 mm glass coverslips were spin-coated with PDMS, treated with UV ozone for 3 minutes, and incubated in 10% APTES in ethanol for 2 hours at 50° C. Coverslips were then rinsed with water and incubated in 2% glutaraldehyde solution in ethanol at room temperature for 1 hour. Coverslips were then rinsed again and inverted onto 150 μL droplets of 50 μg/mL fibronectin in distilled water in a Petri dish, which was then sealed with Parafilm and incubated overnight at 4° C. Cylindrical isotropic stamps were cut from a slab of PDMS using an 8 mm diameter biopsy punch. GFP at 0, 10, 50, 100, or 200 μg/mL was coated onto the isotropic stamps and left for 1.5 hours at room temperature until the solution was dry. The stamps were then briefly dipped into sterile water, air-dried using compressed air, and inverted onto fibronectin-coated coverslips. C3H anti-GFP synNotch mCherry expressing cells were seeded onto patterned coverslips at 650,000 cells per coverslip in a 12-well plate.

To generate micropatterns of GFP, Solidworks was used to design desired patterns (square arrays ranging from 100 μm to 1 mm, concentric circles, aligned rows, and letters), which were then printed into templates using a digital light processing (DLP) 3-D printer (CADworks3D). After 3D printing, the templates were placed in 200 proof isopropyl alcohol overnight to ensure all uncured resin was removed. The templates were then UV cured for 1 hour (back side for 20 minutes, feature side for 40 minutes) to finalize the curing process. PDMS (Sylgard 187) was poured into the templates, desiccated for 30 minutes, and cured overnight in a 65° C. oven. PDMS stamps were then removed from the templates. Microcontact printing with these PDMS stamps was performed the same as with isotropic stamps at 100 μg/mL GFP. For perpendicular row patterns, one stamp with aligned rows was coated with GFP (200 μg/mL) and another similar stamp was coated with mCherry (200 μg/mL). These stamps were manually positioned sequentially in a perpendicular orientation. Coverslips were stored dry at 4° C. until use and incubated in DMEM+10% FBS for a minimum of 1 hour prior to cell seeding. Coverslips were then seeded with C3H anti-GFP synNotch mCherry expressing cells, C3H anti-GFP synNotch MyoD expressing cells, or monoclonal dual receiver cells at a concentration of 650,000 cells per coverslip in a 12-well plate.

For the surfaces in FIG. 5L, PDMS-coated coverslips were treated with UV ozone for 8 minutes, then microcontact printed with a stamp coated with a mixture of 100 μg/mL GFP and 50 μg/mL FN. After patterning, coverslips were incubated in 2% Pluronic in distilled water for 15 minutes at room temperature and rinsed with PBS.

Dual Ligand Patterning with Capillary Microfluidic Device:

Solidworks was used to design a 4-row capillary fluidic device with two disconnected inlets. Shallow channels (100 μm distance from the substrate) were designed to guide protein solutions. These shallow channels were surrounded by 1 mm deep channels, intended as voids. The inverse design was 3-D printed using a DLP printer (CADWorks), which was then replica molded in PDMS. Inlets were created using 1.5 mm biopsy punches and air ventilation punches were created on two opposite sides ends of the device to allow optimal pressure for capillary fluid transfer. Prior to protein patterning, the feature side surface of the device was UV plasma treated for 7 seconds, creating a hydrophilic surface for capillary action. The device was then placed facedown onto tissue culture treated Ibidi 2-wells. GFP (500 μg/mL) and mCherry (1000 μg/mL) were then pipetted into separate inlets and filled their respective shallow channels. The device was then placed into a petri dish and parafilm sealed before overnight incubation at 4° C. The next day, the device was incubated without parafilm at room temperature for 15 minutes. The fluidic device was then carefully removed from the Ibidi wells to minimize liquid disruption. The Ibidi well was left at room temperature for 15 minutes for the protein solutions to dry. The Ibidi well was then UV treated under the biosafety cabinet for 1 hour to sterilize. DMEM with 10% FBS was pipetted into the wells and incubated for 1 hour before cell seeding. Dual-lineage cells were seeded at 1.9×10⁵/cm² and cultured for three days prior to fixation and staining for α-actinin and VEGFR2.

### Data Quantification:

We studied the spatial control dynamics over time by measuring the Pearson's Coefficient on days 2, 5, and 10. This was done using the JACoP plugin in ImageJ by comparing the binary mask to the mCherry channel. Line plots were quantified using the “Plot Profile” feature in ImageJ and normalized to individual images. The number of nuclei in the field of view of each image were counted, and the number of myotubes was counted using ImageJ. Myogenic index was calculated through dividing the number of nuclei within each myotube by the total number of nuclei. The GFP channel was used to determine which nuclei were on and off-pattern. For calculating on-pattern myogenic index, all nuclei outside the GFP pattern were excluded. The sarcomeric α-actinin mask was overlaid on the on-pattern nuclei and used to calculate the myogenic index. Similarly, to quantify off-pattern myogenic index, we excluded all nuclei located within the GFP patterns and used the same sarcomeric α-actinin mask to measure the myogenic index. For Coherency quantification, 200 and 500 μm rows and curves were thresholded using the same methods used to create the myotube mask, except instead of using the thresholded image to create a selection/mask, we quantified the thresholded myotube image itself. The OrientationJ plugin on ImageJ was used to quantify the coherency of all patterns. Since curved rows are not straight, we need to straighten them to get a fair quantification of how the myotubes align with the curves. A fragmented line was drawn manually following the GFP pattern of the curve and used to straighten the myotube threshold image before quantifying the coherency. All data was processed in GraphPad Prism 9 and validated with statistics.

### Plate-Drying of Ligand:

For single-ligand activation, ligands (mCherry and GFP) were prepared at 100 μg/mL in sterile DI water and added at 15 μg/cm². For dual-ligand patterning, 8 uL droplets of ligand at 200 μg/mL in sterile DI water were deposited in distinct regions within each well. Plates were left to dry in the biosafety cabinet overnight, protected from light and then washed once with PBS prior to cell seeding. Anti-mCherry synNotch ETV2-BFP or dual-lineage fibroblasts were seeded at 5-20×10⁴ cells/cm² and cultured for three days prior to flow cytometry analysis or fixation and staining for VEGFR2.

### Staining:

Flow Cytometry: cells were detached using TrypLE (ThermoFisher) and washed once prior to incubation with fluorescently-tagged antibodies in PBS+5% FBS for 30 minutes-1 hour at 4° C. Following, cells were washed twice with PBS+5% FBS and filtered through 35 μm cell strainer prior to analysis with ARIA II.

Following culture, cells were washed once with PBS, fixed with 4% paraformaldehyde or 10% ice-cold methanol for 10 minutes and then washed 3× with PBS for 5 minutes each. Samples were stained immediately or further permeabilized with 0.1% Triton X-100 in PBS for 5-10 minutes and then washed 3× with PBS for 5 minutes. Cells were blocked for 1 hour with 2% BSA at room temperature, then incubated with primary antibodies for 2 hours at room temperature or overnight at 4° C. Following three washes with PBS, samples were incubated with secondary antibodies for one hour at room temperature, then washed again prior to imaging directly or staining nuclei with NucBlue (15 minutes, ThermoFisher) or Nuclear Mask Deep Red (30 min, ThermoFisher). Samples on coverslip were mounted with gold-antifade mounting solution (ThermoFisher).

### Imaging/Microscopy:

Unless otherwise stated, a digital Microscope (Keyence BZ-X) was used to image experiments. Tiling was done with the built-in Keyence software. All images within individual experiments were taken with the same settings (Light strength, exposure, No LUT). BFP, GFP, mCherry, and miRFP signals were captured using the respective Filter cubes: BFP, GFP, TexasRed, Cy5-NX.

### RNA Sequencing

For bulk RNA sequencing analysis, GFP or mCherry solid circle patterns were created via microcontact printing with 8 mm diameter stamps. 5×10⁴ of the following cells were droplet seeded with and without the presence of their respective ligands: C3H parental (no-ligand only), anti-GFP/tTA synNotch that activates mCherry, anti-GFP/tTA synNotch that activates myoD and mCherry, anti-mCherry/Gal4 synNotch that activates ETV2 and BFP, C2C12 cell line (no-ligand only), and BEnd.3 cell line (no-ligand only). To ensure all cells were cultured on the activating ligand, cells were seeded as 50 μL droplets within the borders of the patterned ligands and allowed to adhere for 30 minutes before pipetting in the rest of the culture media. The same cell seeding strategy was performed for conditions without ligands. Cells were cultured for before total RNA extraction using miRNeasy kit per manufacturer's protocol (Qiagen). An RNA cleanup kit was used to further purify/clean the samples (Zymo RNA Clean and Concentrator). RNA samples were then sequenced with an Illumina NovaSeq 6000 (Novogene Corporation Inc).

Fastqc files were trimmed with trimmomatic v0.39 using default settings. Trimmed fastQ files were aligned to GRCm38 reference genome supplemented with custom transgenic sequences using STAR v2.7.10b with default parameters. Transcriptome alignments were quantified using featureCount using the custom gene annotation file combining GENCODE annotation file and transgenes. Gene counts were imported into R and differentially expressed genes were identified with DESEq2 v1.38.3 with p_{adj}=0.05 as the threshold. GO analysis was performed on the differentially expressed genes using the clusterProfiler v4.6.2 package.

### 10× Single-Nuclear RNA Sequencing

For single-nuclear RNA sequencing analysis, patterns were prepared using capillary fluidic device to contain either GFP in both inlets, mCherry in both inlets, or GFP/mCherry in one inlet each to generate a dual-ligand pattern. 3.3×10⁴ dual-lineage fibroblasts were seeded in a 30 uL droplet on top of the patterns, allowed to attach for 30 minutes, prior to adding additional media. Cells were cultured for an additional 3 days before collection. To collect, cells were trypsinized and cells were lysed in IGEPAL CA630-containing lysis buffer for 7 minutes to isolate individual nuclei.

Library construction was performed according to the manufacturer's protocol (10× Genomics single cell 3′ v3.1 protocol). Briefly, after resuspension and counting, 16,000 GCs per experiment were resuspended in master mix and loaded (together with partitioning oil and gel beads), onto each lane of an 8 lane chip G to generate the gel bead-in-emulsion (GEMs). Reverse transcription was primed with an oligonucleotide carrying an Illumina TruSeq R1 read-sequencing primer, a 16 nucleotide 10× cell barcode, a 12 nucleotide UMI, and a 30 nucleotide anchored poly dT sequence. Full length cDNA was amplified from heteroduplex RNA:cDNA using 12 cycles of PCR. The full-length cDNA was cleaned up on SPRIselect beads, and QCed on Qubit and BioAnalyzer. One fourth of the resulting ds cDNA was fragmented and prepared for sample index PCR, with 11 cycles of amplification. After QC, the libraries were pooled and submitted for sequencing on 2 lanes of a 10B 100 flowcell on the Illumina NovaSeqX sequencer, targeting a minimum read depth per cell of 25,000.

FASTQ files were processed with 10× Genomics' Cell Ranger analysis pipelines. The read count matrix generated by CellRanger was then analyzed using Seurat v5.0.2. 32288 genes were detected across no ligand (7877 and 7745 cells tested for replicates), GFP pattern (10539 and 11923 cells tested for replicates), mCherry pattern (10671 and 10068 cells tested for replicates), and Dual Pattern (8710 and 16266 cells tested for replicates). Cells that had unique feature counts with at least 700 genes but no more than 7000 genes and cells that had <55% mitochondrial counts were filtered and normalized based on the feature expression and total expression of each cell. The normalized expression data were then used for subsequent analysis.

Principal component analysis was performed after merging replicates and integrating all the conditions. Highly variable genes in each sample after linear transformation and the first 30 PC scores were used for tSNE analysis to cluster the cells into 12 groups (FindNeighbors and FindClusters functions implemented in the Seurat package, dims=30, resolution=0.4). The marker genes of each cluster were identified using FindAllMarkers or FindMarkers function with default parameters. Clusters were annotated using signature genes and DAVID pathway analysis to identify fibroblast-, muscle-, or endothelial-like cell types across the different conditions. tSNE clusters that were enriched in proliferation, extracellular matrix, or EGF pathways were identified as fibroblasts. Clusters that were enriched in lineage-specific markers, muscle or angiogenesis pathways, were used to identify muscle- and endothelial-like clusters, respectively.

A pseudobulk method was applied to investigate gene expression among different conditions at the population level. Specifically, the raw gene counts of each sample were extracted after filtering. The counts were then aggregated to the sample level and the expression of genes of interest including transgenes were examined across conditions. Statistics:

Individual data points in graphs represent distinct samples. Statistics were calculated in Prism, using Unpaired T-test two-tailed or one-way Anova between groups. *p<0.05, **p=<0.01, ***p=<0.001, ****P=<0.0001

TABLE

| Company | Catalog | Target | Fluorophore | Dilution |
|---|---|---|---|---|
| Cell Signaling | 3739S | Anti-Myc | PE | FC: 1:50 |
| Technologies | | (EQKLISEEDL (SEQ | | |
| | | ID NO: 1)) | | |
| Cell Signaling | 15008S | Anti-FLAG | AlexaFluor | FC: 5 uL/10⁶ |
| Technologies | | (DYKDDDDK (SEQ | 488 | cells |
| | | ID NO: 2)) | | |
| Abcam | ab45688 | Anti-Fibronectin | | IF 1:500 |
| BioLegend | 136401 | Anti-VEGFR2 | | IF: 1:100 |
| BioLegend | 136405 | Anti-VEGFR2 | APC | FC: 1:100 |
| BioLegendSigma | A7811 | Anti-α-Actinin | | IF: 1:200 |
| | | (Sarcomeric) | | |
| BioLegendThermo | A-11030 | Anti-Mouse IgG | AlexaFluor | IF: 1:200 |
| Fisher | | | 546 | |
| Abcam | ab150153 | Anti-Rat IgG | AlexaFluor | IF: 1:200 |
| | | | 488 | |
| ThermoFisher | A-2144 | Anti-Rabbit IgG | AlexaFluor | IF: 1:1000 |
| | | | 647 | |
| ThermoFisher | A-21235 | Anti-Mouse IgG | AlexaFluor | IF 1:200 |
| | | | 647 | |
| ThermoFisher | H10294 | HSC NuclearMask | | IF: 1:250 |
| | | Deep Red | | |
| Abcam | ab228551 | Hoechst 33342 | | IF: 1:20,000 |
| ThermoFisher | R37605 | Hoechst 33342 | | IF: 2drops/mL |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

1. A system or combination, comprising:
(i) a quantity of a ligand, a fusion protein comprising the ligand and an extracellular matrix polypeptide, or a first cell secreting or expressing on cell surface the ligand or the fusion protein, or a combination thereof, wherein the quantity of the ligand, the fusion protein, the cell, or the combination thereof is positioned in a predetermined pattern on a surface of and/or inside a substrate;
(ii) a quantity of a second cell expressing a synthetic receptor, optionally the quantity of the second cell being seeded on a culture surface, wherein the synthetic receptor specifically binds the ligand, the fusion protein, or the first cell via the secreted or expressed ligand or fusion protein; and
(iii) the substrate,
wherein the synthetic receptor comprises synthetic Notch receptor, wherein the synthetic Notch receptor does not bind its naturally-occurring ligand Delta and the synthetic Notch receptor comprises, in covalent linkage:
a) an extracellular domain comprising an antigen-specific targeting domain;
b) a Notch regulatory region comprising a ligand-inducible proteolytic cleavage site; and
c) an intracellular domain, heterologous to the Notch regulatory region, comprising a transcriptional activator, wherein the transcriptional activator replaces a naturally-occurring intracellular notch domain, and
wherein binding of the antigen-specific targeting domain to the ligand, the fusion protein, or the first cell induces cleavage at the Notch regulatory region, thereby releasing the intracellular domain.

2. The system or combination of claim 1, wherein:
(a) the quantity of the ligand, the fusion protein, or the first cell comprises two or more different ligands, two or more different fusion proteins each comprising a different ligand and a same or different extracellular matrix polypeptide, or two or more different populations of cells secreting or expressing on cell surface the different ligands or the different fusion proteins, each being positioned in a different sub-pattern or a same pattern and collectively forming the predetermined pattern on the surface of or inside the substrate;
(b) the quantity of the second cell comprises two or more different populations of cells each expressing a different synthetic Notch receptor capable of specifically binding to the different ligands;
and wherein the quantity of the second cell co-expresses two or more different synthetic Notch receptors, or the system or combination further comprises a quantity of a third cell that expresses a different synthetic Notch receptor compared to that expressed by the quantity of the second cell, wherein the different synthetic Notch receptors differ by the antigen-specific targeting domain, each capable of specifically binding a different one of the two or more different ligands, and differ by the transcriptional activator.

3. The system or combination of claim 1, wherein the substrate comprises an elastomeric stamp, microparticles, a polymeric scaffold or hydrogel, a coverslip, or a microfluidic device; optionally the coverslip having micropatterns of the ligands deposited via the elastomeric stamp or microfluidic device.

4. The system or combination of claim 3, wherein the substrate comprises the elastomeric stamp, and the elastomeric stamp has a patterned elevation of surface, configured for positioning the quantity of the ligand, the fusion protein, the cell, or the combination thereof, wherein the elevation is in the predetermined pattern and having an interspace between neighboring elevations of at least 100 μm.

5. The system or combination of claim 4, wherein the elastomeric stamp is a poly (dimethylsiloxane) (PDMS) stamp, and wherein the elevation is in the pattern having an interspace between neighboring elevations of 200-250, 250-300, 300-350, 350-400, or 400-500 μm.

6. The system or combination of claim 2, wherein the predetermined pattern is an interdigitating pattern, and wherein the two or more different ligands, the two or more different fusion proteins, or the two or more different populations of cells each is positioned in a different sub-pattern and collectively form the interdigitating pattern.

7. The system or combination of claim 2, wherein the two or more different ligands, the two or more different fusion proteins, or the two or more different populations of cells each is positioned in a different sub-pattern with no overlaps.

8. The system or combination of claim 3, further comprising (iv) the culture surface, wherein the quantity of the second cell is cultured on the culture surface, and wherein the substrate comprises the microfluidic device, and the microfluidic device has channels of the predetermined pattern.

9. The system or combination of claim 1, wherein the quantity of the ligand, the fusion protein, the cell, or the combination thereof is chemically conjugated, optionally photocrosslinked, in the predetermined pattern to the surface of or inside the substrate.

10. The system or combination of claim 1, wherein the quantity of the ligand, the fusion protein, the cell, or the combination thereof is adsorbed in the predetermined pattern to the surface of the substrate, optionally via microcontact printing.

11. The system or combination of claim 1, wherein the extracellular matrix polypeptide of the fusion protein is derived from fibronectin, vitronectin, laminin, or collagen.

12. A method for inducing differentiation or transdifferentiation of a cell in a predetermined pattern, comprising:
genetically expressing a synthetic Notch receptor in a cell, wherein the cell contains a gene encoding a differentiation or transdifferentiation agent whose expression is driven by a transcriptional activator, and wherein the synthetic Notch receptor comprises an extracellular domain that specifically binds a ligand, a Notch receptor polypeptide comprising a ligand-inducible proteolytic cleavage site, and an intracellular domain comprising the transcriptional activator;
providing the ligand, the fusion protein, or the sender cell on the surface of or in the substrate in the predetermined pattern; and
contacting the cell expressing the synthetic Notch receptor with the substrate having the predetermined pattern of the ligand, a fusion protein comprising the ligand and an extracellular matrix polypeptide, or a sender cell expressing on sender cell surface the ligand or secreting the ligand or the fusion protein,
wherein binding between the cell expressing the synthetic Notch receptor and the ligand, the fusion protein, or the sender cell induces cleavage of the synthetic Notch receptor, thereby releasing the intracellular domain and causing the transcriptional activator to induce expression of the differentiation or transdifferentiation agent in the cell, thereby differentiating or transdifferentiating the cell in the predetermined pattern, and
wherein the transcriptional activator when uncleaved from the synthetic Notch receptor does not induce expression of the differentiation agent, and the transcriptional activator is substantially uncleaved when the cell is not in contact with the ligand, the fusion protein, or the sender cell.

13. The method of claim 12, wherein the predetermined pattern comprises a feature region and a featureless or spacing region, at least one of which is at a dimension between 1 μm and 1,000 μm, preferably between 10 μm and 500 μm, or about 250 μm; wherein the feature region has immobilized thereto the ligand, the fusion protein, or the sender cell, and the featureless region does not have the ligand, the fusion protein, or the sender cell.

14. The method of claim 12, wherein the providing of the ligand, the fusion protein, or the sender cell in the predetermined pattern comprises photocrosslinking the ligand, the fusion protein, or the sender cell to the substrate in the predetermined pattern.

15. The method of claim 13, wherein the differentiation or transdifferentiation agent comprises a transcription factor.

16. The method of claim 15, wherein the transcription factor comprises myoblast determination protein 1 (MyoD), ETS variant transcription factor 2 (ETV2), atonal bHLH transcription factor 1, NK3 homeobox 1 (NKX3-1), SRY-box transcription factor 9 (SOX-9), or an isoform thereof.

17. The method of claim 12, for differentiation or transdifferentiation of a quantity of the cells into two or more different types of cells, wherein the genetically expressing comprises genetically co-expressing two or more different synthetic Notch receptors in the cells, wherein the two or more different synthetic Notch receptors differ at least by the extracellular domain in specifically binding a different ligand and by the transcriptional activator in driving expression of a different differentiation or transdifferentiation agent, which regulates differentiation or transdifferentiation of the cells into respective type of cells.

18. The method of claim 17, for transdifferentiation of a quantity of fibroblasts into myoblasts and endothelial cells in a predetermined pattern, wherein the method comprises:
genetically co-expressing two different synthetic Notch receptors in a quantity of fibroblasts, optionally embryonic fibroblasts, wherein the two different synthetic Notch receptors each bind a different ligand and thereby releasing a different transcriptional activator, wherein the different transcription activators each drives expression of either one of MyoD or ETV2 in the fibroblasts,
providing the different ligands, different fusion proteins comprising either one of the different ligands and an extracellular matrix polypeptide, or different sender cells secreting or expressing on sender cell surface either one of the different ligands or either one of the different fusion proteins, on the surface of or in the substrate in the predetermined pattern, and
contacting the fibroblasts co-expressing the different synthetic Notch receptors with the substrate,
thereby inducing binding in the predetermined pattern between the fibroblasts and the different ligands, the different fusion proteins, or the different sender cells, and driving expression of the MyoD or the ETV2 in respective fibroblasts, so as to transdifferentiate the fibroblasts into myoblasts and endothelial cells in the predetermined pattern.

19. The method of claim 18, wherein the substrate that the fibroblasts contact with contains an area without a ligand, a fusion protein comprising a ligand, or a sender cell secreting or expressing on sender cell surface a ligand, thereby resulting in at least some of the fibroblasts not being transdifferentiated and forming a co-culture containing the un-transdifferentiated fibroblasts, myoblasts, and endothelial cells.

20. A co-culture of cells, comprising myoblasts and endothelial cells in a predetermined pattern made from the method of claim 18.

21. A co-culture of cells, comprising fibroblasts, myoblasts, and endothelial cells in a predetermined pattern made from the method of claim 19.

22. The co-culture system of claim 20, wherein the co-culture of cells is in the form of a blood vessel-like tissue structure.

23. A kit, comprising a substrate; and a ligand or a fusion protein comprising the ligand and an extracellular matrix polypeptide; and optionally further a polynucleotide encoding a synthetic Notch receptor, wherein the synthetic Notch receptor does not bind its naturally-occurring ligand Delta and instead binds to the ligand.

24. The kit of claim 23, wherein the ligand or the fusion protein is deposited in a micropattern on or is the substrate, and the substrate comprises an elastomeric stamp, microparticles, a polymeric scaffold or hydrogel, a coverslip, or a microfluidic device.
